(19) United States
(12) Patent Application Publication
(10) Pub. No.: US 2023/0000876 A1
(43) Pub. Date: Jan. 5, 2023

(54) TREATING CANCERS WITH A CYCLIN-DEPENDENT KINASE INHIBITOR

(71) Applicant: Nuvation Bio Inc., New York, NY (US)

(72) Inventors: Teeru BIHANI, Long Island City, NY (US); Gary HATTERSLEY, Stow, MA (US); Chris P. MILLER, San Mateo, CA (US); Hitisha PATEL, Laurel, MD (US); Sergey YURASOV, San Fransciso, CA (US); Yan ZHANG, Hockessin, DE (US)

(21) Appl. No.: 17/835,827

(22) Filed: Jun. 8, 2022

Related U.S. Application Data
(60) Provisional application No. 63/297,534, filed on Jan. 7, 2022.
(60) Provisional application No. 63/208,962, filed on Jun. 9, 2021.

(51) Int. Cl. A61K 31/538; A61P 35/04; A61K 31/565; A61K 39/395; A61K 31/517; A61K 31/4166; A61K 31/495

(52) U.S. Cl. CPC A61K 31/538; A61P 35/04; A61K 31/565; A61K 39/39558; A61K 31/517; A61K 31/4166; A61K 31/495

(57) ABSTRACT

The present disclosure relates to methods of treating cancers, including metastasized cancers by administering a potent CDK 2/4/6 inhibitor of the formula (I), or a pharmaceutically acceptable salt thereof, to a patient in need thereof.

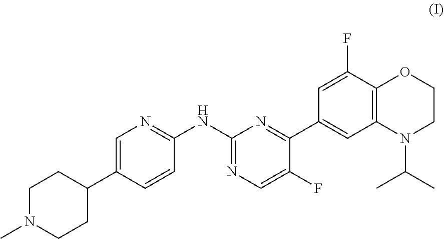 [SMILES: CC(C)N1CCOc2c(F)cc(-c3nc(Nc4ccc(C5CCN(C)CC5)cn4)ncc3F)cc21]

### CROSS-REFERENCE TO RELATED APPLICATIONS

This application claims priority to U.S. Provisional Application No. 63/208,962, filed Jun. 9, 2021, and U.S. Provisional Application No. 63/297,534, filed Jan. 7, 2022, the content of each of which is hereby incorporated herein by reference in its entirety.

### FIELD

Provided herein are methods of treating cancers, including metastasized cancers, in a subject in need thereof, comprising administering an inhibitor of CDK 2/4/6.

### BACKGROUND

The cell cycle is a period between the successive divisions of a cell. During this period, the contents of the cell must be accurately replicated. The processes that permit the cell to divide are very precisely controlled by a multitude of enzymatic reactions amongst which the protein kinase-triggered protein phosphorylation plays a major role. In eukaryotes, there are four main stages/phases of cell cycle namely the Gap-1 (G1) phase, Synthesis (S) phase, Gap-2 (G2) and Mitosis (M) phases. An extended phase of Gap-1 phase is coined as Gap-0 (G0) phase or Resting phase (see Cancers 2014, 6, 2224-2242, which is hereby incorporated by reference).

Uncontrolled proliferation is the hallmark of cancer and other proliferative disorders and abnormal cell cycle regulation is, therefore, common in these diseases. Cyclin-dependent kinases (CDK) constitute a heterodimeric family of serine/threonine protein kinases involved in cell cycle and transcription. They include two main groups: cell cycle CDK and transcriptional CDK. The functionality of CDK depends on specific interactions with regulatory proteins named cyclins which form heterodimeric complexes with their partners. These complexes are important regulators of the cellular processes, especially in the cell cycle progression.

The human proteome contains 20 CDK along with 29 cyclins. CDK1, CDK2, CDK4 and CDK6 are generally considered cell cycle CDK, whereas CDK7, CDK8, CDK9 and CDK11 are mainly involved in transcription regulation (see Genome Biol 2014; 15(6):122, Nat Cell Biol 2009; 11(11):1275-6, which is hereby incorporated by reference). CDK5 is the prototype of atypical CDK: it is activated by the non-cyclin proteins p35 (or Cdk5R1) and p39 (or Cdk5R2) and has unique post-mitotic functions in neuronal biology, angiogenesis and cell differentiation. Proliferative signals induce the transition from the G0 or G1 phases into S phase through the activation of the structurally related CDK4 and CDK6 [see Development, 2013; 140 (15):3079-93, Biochem Pharmacol 2012; 84(8):985-93, and Nature 2014; 510(7505):393-6, each of which is hereby incorporated by reference]. The binding of cyclin D to CDK4 and to CDK6 promotes the phosphorylation of the transcriptional repressor retinoblastoma protein (RB1).

CDK hyperactivity is often observed in cancer, reflecting their prominent role in cell cycle and transcription regulation. In cancer cells, the process of cell division becomes unregulated, resulting in uncontrolled growth that leads to the development of a tumor. A number of mechanisms contribute to the dysregulation of the cell cycle in malignant cells, including the amplification and hyperactivity of CDK4/6, or their genomic instability, which might cause CDK4/6 to become oncogenic drivers of cell replication. Usurping these mechanisms, cancer cells can continue to replicate by triggering the G1 to S phase transition. This process appears to be facilitated by a shortening of the G1 phase. In a cancer cell, CDK4/6 antagonizes intrinsic tumor suppression mechanisms including cell senescence and apoptosis, which further augments the growth of a tumor. Cancer cells also upregulate other CDK and cyclins and decrease suppressive mechanisms such as intrinsic CDK inhibitors and tumor suppressor proteins. The overall effect of this type of cell cycle dysregulation is malignant cell proliferation and the development of cancer (see Clinical Breast Cancer, 2016; 16(1):8-17, which is hereby incorporated by reference).

First generation dual CDK4/6 inhibitors such as palbociclib, ribociclib and abemaciclib have been found to be effective in the treatment of particular cancers, including breast cancer. However, patients often develop resistance to dual CDK4/6 inhibitors, which limits the overall effectiveness of the dual inhibitor. One rationale for resistance is through CDK2 signaling, which allows cancers to bypass CDK 4/6 inhibition. CDK2 activity is known to drive tumorigenesis in multiple solid tumors including particular types of brain cancer, prostate cancer and breast cancer.

The development of therapies, including monotherapies, for treatment of proliferative disorders using a therapeutic targeted generically at CDK, or specifically at inhibition of CDK2, CDK4 and CDK6, is therefore potentially highly desirable. U.S. Patent Publication No. US 2019/0248774 A1, which is hereby incorporated by reference, discloses 5-fluoro-4-(8-fluoro-4-isopropyl-3,4-dihydro-2H-benzo[b][1,4]oxazin-6-yl)-N-(5-(1-methylpiperidin-4-yl)pyridin-2-yl)pyrimidin-2-amine (hereinafter the “Compound of formula (I)” or “Compound (I)” or “formula (I)”) having the structure shown below, which is a potent CDK2/4/6 inhibitor. It is understood that the other compounds disclosed in US 2019/0248774 may be used in the methods or combinations disclosed herein as well.

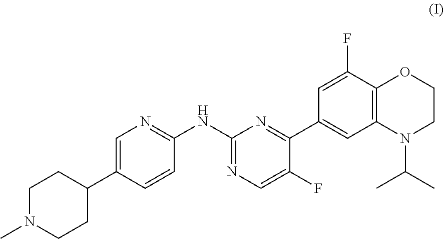 [SMILES: CC(C)N1CCOc2c(F)cc(-c3nc(Nc4ccc(C5CCN(C)CC5)cn4)ncc3F)cc21]

As disclosed herein, the compound of formula (I) overcomes problems associated with first generation CDK 4/6 inhibitors and is highly effective in treating cancers that are resistant to known anti-cancer therapies.

### BRIEF SUMMARY

The disclosure provides methods of treating cancers, including metastasized cancers, in a subject in need thereof, comprising administering to the subject a compound of the formula (I)

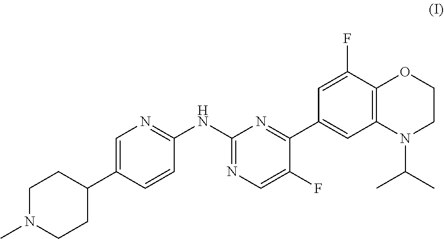 [SMILES: CC(C)N1CCOc2c(F)cc(-c3nc(Nc4ccc(C5CCN(C)CC5)cn4)ncc3F)cc21]

or a pharmaceutically acceptable salt thereof.

In one aspect, provided herein are methods of treating breast cancer (e.g., metastatic breast cancer (mBC)) in a subject in need thereof, comprising administering to the subject a compound of formula (I) or a pharmaceutically acceptable salt thereof. In some embodiments, the breast cancer has metastasized to the brain. In some embodiments, the cancer is a metastatic triple negative breast cancer. In further embodiments, the metastatic triple negative breast cancer has metastasized to the brain. In some embodiments, the breast cancer (e.g., mBC) is hormone receptor-positive (HR+). In some such embodiments, the HR+ breast cancer is estrogen receptor-positive (ER+) breast cancer. In some embodiments, the breast cancer (e.g., mBC) is HR+ and HER2− (e.g., ER+ and HER2−). In some embodiments, the breast cancer (e.g., mBC) is HR+ and HER2+(e.g., ER+ and HER2+). In some embodiments, the breast cancer (e.g., mBC) is HR− and HER2+. In some embodiments, the compound of formula (I), or a pharmaceutically acceptable salt thereof, is administered in combination with an anti-estrogen therapy. For instance, the anti-estrogen therapy is a selective estrogen receptor degrader (SERD), selective estrogen receptor modulator (SERM), estrogen receptor downregulator (ERD) or an aromatase inhibitor. In some embodiments the anti-estrogen therapy is fulvestrant, AZD9833, amcenestrant, giredestrant or OP1250. In other embodiments, the compound of formula (I), or a pharmaceutically acceptable salt thereof, is administered in combination with an HER2 targeted therapy (e.g., trastuzumab or tucatinib or a combination thereof).

In another aspect, provided herein is a method of treating prostate cancer in a subject in need thereof, comprising administering to the subject a compound of formula (I), or a pharmaceutically acceptable salt thereof. In some embodiments, provided herein is a method of treating castration-resistant prostate cancer (CRPC) in a subject in need thereof, comprising administering to the subject a compound of formula (I), or a pharmaceutically acceptable salt thereof. In one embodiment, the CRPC is metastatic castration-resistant prostate cancer (mCRPC). In some embodiments, the mCRPC of the patient has metastasized to the brain. In some embodiments, the compound of formula (I), or a pharmaceutically acceptable salt thereof, is administered in combination with an anti-androgen therapy. In some such embodiments, the anti-androgen therapy is enzalutamide.

In another aspect, provided herein is a method of treating glioma in a subject in need thereof, comprising administering to the subject a compound of formula (I), or a pharmaceutically acceptable salt thereof. In some embodiments, provided herein is a method of treating high-grade glioma in a subject in need thereof, comprising administering to the subject a compound of formula (I), or a pharmaceutically acceptable salt thereof. In some embodiments, the high-grade glioma is glioblastoma. In other embodiments, the high-grade glioma is characterized by a CDKN2A mutation. In certain embodiments, the high grade glioma overexpresses CDK2 and/or cyclin E.

In another aspect, provided herein is a method of treating brain metastases in a subject in need thereof, comprising administering to the subject a compound of formula (I), or a pharmaceutically acceptable salt thereof. It has been found that the compound of formula (I), or a pharmaceutically acceptable salt thereof, has unexpectedly high concentrations in the brain following administration (e.g., oral administration). Therefore, the compound of formula (I), or a pharmaceutically acceptable salt thereof, is capable of preventing the growth and survival of metastasizing cancer cells in the brain of a subject in need thereof.

In another aspect, provided herein is a method of treating cancer in a subject in need thereof, comprising administering to the subject a compound of formula (I), or a pharmaceutically acceptable salt thereof, wherein the compound of formula (I), or a pharmaceutically acceptable salt thereof, is administered according to a dosing schedule that does not significantly inhibit CDK1.

### BRIEF DESCRIPTION OF THE DRAWINGS

FIG. 1 shows tumor volume growth curves of female Balb/c nude mice bearing MCF-7 tumors following administration of the compound of formula (I) either alone or in combination with fulvestrant. Data points represented as mean±SEM.

FIG. 2 shows tumor growth curves of female Balb/c nude mice bearing MCF-7 tumors following administration of the compound of formula (I) either alone or in combination with an ascending dose of fulvestrant. Data points represented as mean±SEM.

FIG. 3 shows tumor weight curves of female Balb/c nude mice bearing MCF-7 tumors following administration of the compound of formula (I) either alone or in combination with an ascending dose of fulvestrant. Data points represented as mean±SEM.

FIG. 4 shows tumor volume growth curves of male Balb/c nude mice bearing 22Rv1 tumors following administration of the compound of formula (I) either alone or in combination with enzalutamide.

FIG. 5 shows the effect of tumor weight on PG-D24/D25 of male Balb/c nude mice bearing 22Rv1 tumors following administration of the compound of formula (I) either alone or in combination with enzalutamide.

FIGS. 6A-6G show inhibition curves for the compound of formula (I) and temozolomide in cell viability assays in seven different metastatic cell lines. FIG. 6A shows the inhibition curves in a cell viability assays using the ACNH cell line. FIG. 6B shows the inhibition curves in a cell viability assay using the H1975 cell line. FIG. 6C shows the inhibition curves in a cell viability assays using the MOLT-4 cell line. FIG. 6D shows the inhibition curves in a cell viability assays using the PC-9 cell line. FIG. 6E shows the inhibition curves in a cell viability assays using the NCI-H460 cell line. FIG. 6F shows the inhibition curves in a cell viability assays using the KM12 cell line. FIG. 6G shows the inhibition curves in a cell viability assays using the Nalm-6 cell line.

FIG. 7 shows the mean plasma and brain concentration-time curves for the compound of formula (I) following oral administration.

FIG. 8 shows survival curves for female BALB/c nude mice bearing U-87MG-luc orthotopic intracranial tumors following administration of the compound of formula (I) or temozolomide.

FIG. 9 shows tumor volume growth curves of female CB17 SCID mice bearing U-118MG xenograft tumors following administration of the compound of formula (I) or temozolomide. Data points represented as mean±SEM.

FIG. 10 shows tumor weight of female CB17 SCID mice bearing U-118MG xenograft tumors following administration of the compound of formula (I) or temozolomide. Data points represented as mean±SEM.

FIG. 11 shows tumor volumes of mice in male Balb/c nude mice bearing ST2347 PDX tumor model following administration of the compound of formula (I) either alone or in combination with enzalutamide.

FIG. 12 shows tumor weight curves of female Balb/c nude mice bearing HER2+ tumors following administration of the compound of formula (I) either alone or in combination with one or more HER2 inhibitors.

### DETAILED DESCRIPTION

### Definitions

As used herein, unless clearly indicated otherwise, use of the terms “a”, “an” and the like refers to one or more.

As used herein, reference to “about” a value or parameter herein includes (and describes) embodiments that are directed to that value or parameter per se. For example, description referring to “about X” includes description of “X”. As used herein, and unless otherwise specified, the terms “about” and “approximately,” when used in connection with doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in this context, contemplate a dose, amount, or weight percent within 20%, within 15%, within 10%, within 5%, within 4%, within 3%, within 2%, within 1%, or within 0.5% of the specified dose, amount, or weight percent.

Unless clearly indicated otherwise, “a subject” as used herein intends a mammal, including but not limited to a primate, human, bovine, horse, feline, canine, or rodent. In one variation, the subject is a human.

As used herein, “treatment” or “treating” is an approach for obtaining beneficial or desired results including clinical results. Beneficial or desired results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, delaying the occurrence or recurrence of the disease, delay or slowing the progression of the disease, ameliorating the disease state, providing a remission (whether partial or total) of the disease, decreasing the dose of one or more other medications required to treat the disease, enhancing effect of another medication, delaying the progression of the disease, increasing the quality of life, and/or prolonging survival. The methods of the invention contemplate any one or more of these aspects of treatment.

In reference to cancers or other unwanted cell proliferation, beneficial or desired results include shrinking a tumor (reducing tumor size); decreasing the growth rate of the tumor (such as to suppress tumor growth); reducing the number of cancer cells; inhibiting, retarding or slowing to some extent and preferably stopping cancer cell infiltration into peripheral organs; inhibiting (slowing to some extent and preferably stopping) tumor metastasis; inhibiting tumor growth; and/or relieving to some extent one or more of the symptoms associated with the cancer. In some embodiments, beneficial or desired results in reference to cancers include preventing or delaying recurrence, such as of unwanted cell proliferation. Any of the methods of treatment or treating a cancer detailed herein, such as treatment methods for metastatic breast cancer (mBC), metastatic castration-resistant prostate cancer (mCRPC), and a high-grade glioma, in some embodiments comprise any one or more of shrinking a tumor (reducing tumor size); decreasing the growth rate of the tumor (such as to suppress tumor growth); reducing the number of cancer cells; inhibiting, retarding or slowing to some extent and preferably stopping cancer cell infiltration into peripheral organs; inhibiting (slowing to some extent and preferably stopping) tumor metastasis; inhibiting tumor growth; and relieving to some extent one or more of the symptoms associated with the cancer.

As used herein, by “combination therapy” is meant a therapy that includes two or more different compounds or therapeutic agents. Thus, in one aspect, a combination therapy comprising a compound detailed herein and another compound or therapeutic agent is provided. The different therapeutic compounds can be administered either in the same pharmaceutical composition or in separate compositions, either concomitantly or sequentially. The therapeutic agents can be administered by the same route of administration or by different routes of administration. In various embodiments, treatment with a combination therapy may result in an additive or even synergistic (e.g., greater than additive) result compared to administration of a single compound of the disclosure alone. In some embodiments, a lower amount of each compound is used as part of a combination therapy compared to the amount generally used for individual therapy. Preferably, the same or greater therapeutic benefit is achieved using a combination therapy than by using any of the individual compounds alone. In some embodiments, the same or greater therapeutic benefit is achieved using a smaller amount (e.g., a lower dose or a less frequent dosing schedule) of a compound in a combination therapy than the amount generally used for individual compound or therapy. Preferably, the use of a small amount of compound results in a reduction in the number, severity, frequency and/or duration of one or more side-effects associated with the compound.

The terms “inhibit,” “inhibiting,” and “inhibition” refer to the ability of a compound (e.g., a compound of formula (I)) to reduce or block a particular function, activity or level. In some embodiments, “inhibition” refers to the ability of a compound of formula (I), or a pharmaceutically acceptable salt thereof, to block binding to a CDK. As described herein, inhibition of CDK 2/4/6 results in the slowing, halting, or reversing the growth or progression of a disease, infection, condition, or group of cells. The inhibition can be greater than about 30%, 40%, 50% 60%, 80%, 90%, 95%, or 99%, for example, compared to the growth or progression that occurs in the absence of the treatment or contacting.

As used herein, the term “effective amount” intends such amount of a compound of formula (I), or a pharmaceutically acceptable salt thereof, which should be effective in a given therapeutic form. As is understood in the art, an effective amount may be in one or more doses, i.e., a single dose or multiple doses may be required to achieve the desired treatment endpoint. An effective amount may be considered in the context of administering one or more therapeutic agents (e.g., a compound or formula (I)), and a single agent may be considered to be given in an effective amount if, in conjunction with one or more other agents, a desirable or beneficial result may be or is achieved. Suitable doses of any of the co-administered compounds may optionally be lowered due to the combined action (e.g., additive or synergistic effects) of the compounds.

As used herein, a “therapeutically effective amount” refers to an amount of a compound of formula (I), or a pharmaceutically acceptable salt thereof, sufficient to produce a desired therapeutic outcome.

As used herein, “unit dosage form” refers to physically discrete units, suitable as unit dosages, each unit containing a predetermined quantity of active ingredient calculated to produce the desired therapeutic effect in association with the required pharmaceutical carrier. Unit dosage forms may contain a single or a combination therapy.

As used herein, the term “controlled release” refers to a drug-containing formulation or fraction thereof in which release of the drug is not immediate, i.e., with a “controlled release” formulation, administration does not result in immediate release of the drug into an absorption pool. The term encompasses depot formulations designed to gradually release the drug compound of formula (I), or a pharmaceutically acceptable salt thereof, over an extended period of time. Controlled release formulations can include a wide variety of drug delivery systems, generally involving mixing the drug compound with carriers, polymers or other compounds having the desired release characteristics (e.g., pH-dependent or non-pH-dependent solubility, different degrees of water solubility, and the like) and formulating the mixture according to the desired route of delivery (e.g., coated capsules, implantable reservoirs, injectable solutions containing biodegradable capsules, and the like).

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide and the like. Further examples of pharmaceutically acceptable salts include those listed in Berge et al., Pharmaceutical Salts, J. Pharm. Sci. 1977 January; 66(1):1-19, which is hereby incorporated by reference. Pharmaceutically acceptable salts can be prepared in situ in the manufacturing process, or by separately reacting a purified compound of the disclosure in its free acid or base form with a suitable organic or inorganic base or acid, respectively and isolating the salt thus formed during subsequent purification. It should be understood that a reference to a pharmaceutically acceptable salt includes the solvent addition forms or crystal forms thereof, particularly solvates or polymorphs. Solvates contain either stoichiometric or non-stoichiometric amounts of a solvent and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability and solubility. Various factors such as the recrystallization solvent, rate of crystallization and storage temperature may cause a single crystal form to dominate.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

It is understood that aspects and embodiments described herein as “comprising” include “consisting of” and “consisting essentially of” embodiments.

### Methods of Use

Compounds and compositions detailed herein may be used in methods of administration and treatment as provided herein. In some embodiments of the methods detailed herein, the methods comprise administration of a compound of the formula (I), or a pharmaceutically acceptable salt thereof, as a monotherapy to treat a cancer (e.g., breast cancer, prostate cancer, or glioma). In some embodiments of the methods detailed herein, the methods comprise administration of a compound of the formula (I), or a pharmaceutically acceptable salt thereof, as a monotherapy to treat breast cancer, prostate cancer or high-grade glioma. In other embodiments, the methods comprise administration of a compound of the formula (I), or a pharmaceutically acceptable salt thereof, as part of a combination therapy, as described herein.

Provided herein are methods of treating cancers, including metastasized cancers or high-grade gliomas, in a subject in need thereof, comprising administering to the subject a compound of the formula (I), or a pharmaceutically acceptable salt thereof. The compound of the formula (I), or a pharmaceutically acceptable salt thereof, can be used to treat cancers that are not responsive to or have developed a primary or secondary resistance to other anti-cancer agents. For instance, the compound of formula (I), or a pharmaceutically acceptable salt thereof, can be used to treat cancers that are unresponsive or have grown resistant to other CDK inhibitors. In some embodiments, the compound of formula (I), or a pharmaceutically acceptable salt thereof, can be used to treat cancers that are unresponsive or have become resistant to other CDK 4/6 inhibitors such as, but not limited to, palbociclib (Ibrance®), ribociclib (Kisqali®) and abemaciclib (Verzenio®). In some embodiments, the compound of formula (I), or a pharmaceutically acceptable salt thereof, can be used to treat cancers that are unresponsive to or have become resistant to standard of care treatments for a particular type of cancer, as set forth herein.

In some embodiments, the cancer (e.g., advanced or metastasized cancer) has a mutation in the Estrogen Receptor 1 (ESR1) gene that encodes Estrogen receptor alpha (ERα). In some embodiments, the mutation is at an amino acid selected from A593, S576, G557, R555, L549, A546, E542, L540, D538, Y537, L536, P535, V534, V533, N532, K531, C530, H524, E523, M522, R503, L497, K481, V478, R477, E471, S463, F461, S432, G420, V418, D411, L466, S463, L453, G442, M437, M421, M396, V392, M388, E380, G344, S338, L370, S329, K303, A283, S282, E279, G274, K252, R233, P222, G160, N156, P147, G145, F97, N69, A65, A58 and S47. In some embodiments, the mutation is at an amino acid selected from D538 and Y537. In some embodiments, the mutation is at D538. In some embodiments, the mutation is at Y537. In some embodiments, the mutation is selected from K303R, D538G, Y537S, E380Q, Y537C, Y537N, A283V, A546D, A546T, A58T, A593D, A65V, C530L, D411H, E279V, E471D, E471V, E523Q, E542G, F461V, F97L, G145D, G160D, G274R, G344D, G420D, G442R, G557R, H524L, K252N, K481N, K531E, L370F, L453F, L466Q, L497R, L536H, L536P, L536Q, L536R, L540Q, L549P, M388L, M396V, M421V, M437I, M522I, N156T, N532K, N69K, P147Q, P222S, P535H, R233G, R477Q, R503W, R555H, S282C, S329Y, S338G, S432L, S463P, S47T, S576L, V392I, V418E, V478L, V533M, V534E, Y537D and Y537H. In some embodiments, the mutation is Y537S.

In some embodiments, the cancer (e.g., advanced or metastasized cancer) in the subject has one or more mutations or amplification or overexpression of the genes encoding cyclins or of the genes encoding the CDK or loss of endogenous INK4 inhibitors by gene deletion, mutation, or promoter hypermethylation, or other genetic events leading to overactivity of one or more of CDK2, CDK4, and CDK6. In some embodiments, the cancer in the subject has one or more mutations or amplification or overexpression of the genes encoding cyclins or of the genes encoding the CDK or loss of endogenous INK4 inhibitors by gene deletion, mutation, or promoter hypermethylation, or other genetic events leading to overactivity of CDK4/6 and CDK2.

In some embodiments, the cancer (e.g., advanced or metastasized cancer) in the subject has one or more mutations or amplification or overexpression of the genes encoding cyclins or of the genes encoding the CDK or loss of endogenous INK4 inhibitors by gene deletion, mutation, or promoter hypermethylation, or other genetic events leading to overactivity of one or more of CDK2, CDK4, and CDK6. In some embodiments, the cancer in the subject has one or more mutations or amplification or overexpression of the genes encoding cyclins or of the genes encoding the CDK or loss of endogenous INK4 inhibitors by gene deletion, mutation, or promoter hypermethylation, or other genetic events leading to overactivity of CDK4/6 and CDK2.

In some embodiments, provided is a method of treating a cancer (e.g., advanced or metastasized cancer) in an subject, comprising (a) selecting the subject for treatment based on (i) the presence of phosphorylation of the retinoblastoma (Rb) protein in the cancer, or (ii) presence of mutations or amplification or overexpression of CDK2, CDK4 or CDK6 in the cancer, and administering an effective amount of a compound of formula (I) to the subject. In some embodiments, the cancer is assayed for the expression of phosphorylated Rb. In some embodiments, the cancer is assayed for the expression of CDK2, CDK4 or CDK6. In some embodiments, the CDK2, CDK4 or CDK6 gene of the cancer is sequenced to detect the one or more mutations or amplifications. In some embodiments, the CDK2, CDK4 or CDK6 gene is sequenced by biopsying the cancer and sequencing the CDK2, CDK4 or CDK6 gene from the biopsied cancer. In some embodiments, the CDK2, CDK4 or CDK6 gene is sequenced by sequencing circulating-tumor DNA (ctDNA) from the subject. In some embodiments, the tumor is biopsied for upregulation of cyclin 2E wherein elevated levels of cyclin 2E can indicate resistance to CDK4/CDK6 inhibitor treatment.

The compounds of formula (I), or a pharmaceutically acceptable salt thereof, can be administered to a cancer patient at a dose that simultaneously inhibits CDK2, CDK4 and CDK6. In particular embodiments, the compound of formula (I), or a pharmaceutically acceptable salt thereof, can be administered to a cancer patient at a dose that simultaneously inhibits CDK2, CDK4 and CDK6 with less inhibition of CDK1. Therefore, the compound of formula (I), or a pharmaceutically acceptable salt thereof, can be administered to a cancer patient at doses that effectively treats cancer with reduced side effects, including side effects associated with too much CDK1 inhibition. Accordingly, one aspect of the disclosure provides methods of treating cancer in a subject in need thereof, comprising administering to the subject a compound of formula (I), or a pharmaceutically acceptable salt thereof, according to a dosing schedule that does not significantly inhibit CDK1.

In some embodiments, the compound of formula (I), or a pharmaceutically acceptable salt thereof, is administered at a dose that simultaneously inhibits CDK2, CDK4, and CDK6 greater than about 50% and inhibits CDK1 less than about 20%. In other embodiments, the compound of formula (I), or a pharmaceutically acceptable salt thereof, is administered at a dose that simultaneously inhibits CDK2, CDK4, and CDK6 greater than about 50% and inhibits CDK1 less than about 10%. In some embodiments, the compound of formula (I), or a pharmaceutically acceptable salt thereof, is administered at a dose that simultaneously inhibits CDK2, CDK4, and CDK6 greater than about 75% and inhibits CDK1 less than about 20%. In some embodiments, the compound of formula (I), or a pharmaceutically acceptable salt thereof, is administered at a dose that simultaneously inhibits CDK2, CDK4, and CDK6 greater than about 75% and inhibits CDK1 less than about 10%. In some embodiments, the compound of formula (I), or a pharmaceutically acceptable salt thereof, is administered at a dose that simultaneously inhibits CDK2, CDK4, and CDK6 greater than about 90% and inhibits CDK1 less than about 20%. In some embodiments, the compound of formula (I), or a pharmaceutically acceptable salt thereof, is administered at a dose that simultaneously inhibits CDK2, CDK4, and CDK6 greater than about 90% and inhibits CDK1 less than about 10%. In some embodiments, the compound of formula (I), or a pharmaceutically acceptable salt thereof, is administered at a dose that simultaneously inhibits CDK2, CDK4, and CDK6 greater than about 50% (e.g., 60%, 70%, 80%, 90%, 95% or 99%) and inhibits CDK1 less than about 1%. In some such embodiments, the compound of formula (I), or a pharmaceutically acceptable salt thereof, is administered at a dose that does not inhibit CDK1.

In some embodiments, the compound of formula (I), or a pharmaceutically acceptable salt thereof, and compositions described herein, cause G₁-S cell cycle arrest in a cancer cell. In some embodiments, arrested cells enter a state of apoptosis. In some embodiments, arrested cells enter a state of senescence. In some embodiments, provided herein is a method of causing G₁-S checkpoint arrest in a cell comprising administering an effective amount of a compound of formula (I), or a pharmaceutically acceptable salt thereof, to the cell. In some embodiments, the G₁-S cell cycle arrest occurs in about 40% or more, about 50% or more, about 60% or more, about 70% or more, about 80% or more, about 85% or more, about 90% or more, about 95% or more, about 96% or more, about 97% or more, about 98% or more, or about 99% or more of cancer cells in a cell population. In some embodiments, the G₁-S cell cycle arrest occurs in up to about 99%, up to about 98%, up to about 97%, up to about 96%, up to about 95%, up to about 90%, up to about 85%, or up to about 80% of cancer cells in the cell population.

As set forth herein, the compound of formula (I), or a pharmaceutically acceptable salt thereof, can be administered in combination with other anti-cancer agents. The additional anti-cancer agents will, in part, depend on the cancer being treated. In some embodiments, as described herein, the anti-cancer agent is an anti-estrogen therapy (e.g., fulvestrant). In other embodiments, the anti-cancer agent is a HER2 targeted therapy (e.g., trastuzumab or tucatinib or a combination thereof). In some embodiments, the anti-cancer agent is an anti-estrogen therapy (e.g., fulvestrant or elacestrant), a HER2 targeted therapy (e.g., trastuzumab or tucatinib), or a combination thereof. For example, the anti-cancer agent may be a combination of fulvestrant and trastuzumab, a combination of trastuzumab and tucatinib, or a combination of fulvestrant and tucatinib. In other embodiments, the anti-cancer agent is administered in combination with an anti-androgen therapy (e.g., enzalutamide). In other embodiments, the anti-cancer agent is an alkylating agent (e.g., temozolomide (TMZ)).

Breast Cancer

In one aspect, provided herein are methods of treating breast cancer in a subject in need thereof, comprising administering to the subject a compound of formula (I) or a pharmaceutically acceptable salt thereof. In some embodiments, the method of treating breast cancer comprises reduction of tumor volume. In some embodiments, the method of treating breast cancer comprises tumor regression. In some embodiments, the breast cancer is metastatic hormone receptor-positive (HR+) breast cancer. In embodiments where the breast cancer is HR+, the breast cancer can be estrogen receptor positive (ER+), progesterone receptor positive (PR+), or both estrogen and progesterone receptor positive (ER+/PR+). In particular embodiments, the HR+ breast cancer is ER+ breast cancer. In other embodiments, the breast cancer is hormone receptor-negative breast cancer (HR−). In other embodiments, the breast cancer is human epidermal growth factor receptor 2-negative (HER2−) breast cancer. In other embodiments, the breast cancer is human epidermal growth factor receptor 2-positive (HER2+) breast cancer. In other embodiments, the breast cancer is HR+HER2− (e.g., ER+HER2−) breast cancer. In other embodiments, the breast cancer is HR+HER2+ (e.g., ER+HER2+) breast cancer. In other embodiments, the breast cancer is HR−HER2+ breast cancer. In other embodiments, the breast cancer is triple negative breast cancer. In any of the preceding embodiments, the cancer has a mutation in the CDKN2A gene. In any of the preceding embodiments, the cancer has a mutation in the ESR1 gene. In some embodiments, the mutation is at an amino acid selected from A593, S576, G557, R555, L549, A546, E542, L540, D538, Y537, L536, P535, V534, V533, N532, K531, C530, H524, E523, M522, R503, L497, K481, V478, R477, E471, S463, F461, S432, G420, V418, D411, L466, S463, L453, G442, M437, M421, M396, V392, M388, E380, G344, S338, L370, S329, K303, A283, S282, E279, G274, K252, R233, P222, G160, N156, P147, G145, F97, N69, A65, A58 and S47. In some embodiments, the mutation is at an amino acid selected from D538 and Y537. In some embodiments, the mutation is at D538. In some embodiments, the mutation is at Y537. In some embodiments, the mutation is selected from K303R, D538G, Y537S, E380Q, Y537C, Y537N, A283V, A546D, A546T, A58T, A593D, A65V, C530L, D411H, E279V, E471D, E471V, E523Q, E542G, F461V, F97L, G145D, G160D, G274R, G344D, G420D, G442R, G557R, H524L, K252N, K481N, K531E, L370F, L453F, L466Q, L497R, L536H, L536P, L536Q, L536R, L540Q, L549P, M388L, M396V, M421V, M437I, M522I, N156T, N532K, N69K, P147Q, P222S, P535H, R233G, R477Q, R503W, R555H, S282C, S329Y, S338G, S432L, S463P, S47T, S576L, V392I, V418E, V478L, V533M, V534E, Y537D and Y537H. In some embodiments, the mutation is Y537S.

In another aspect, provided herein are methods of treating metastatic breast cancer (mBC) in a subject in need thereof, comprising administering to the subject a compound of formula (I) or a pharmaceutically acceptable salt thereof. In some embodiments, provided herein are methods of treating metastatic hormone receptor-positive breast cancer (HR+mBC) (e.g., metastatic hormone receptor-positive human epidermal growth factor receptor 2-negative cancer (HR+HER2−mBC) in a subject in need thereof, comprising administering to the subject a compound of formula (I) or a pharmaceutically acceptable salt thereof. In embodiments where the metastatic breast cancer is HR+, the metastatic breast cancer can be estrogen receptor positive (ER+), progesterone receptor positive (PR+), or both estrogen and progesterone receptor positive (ER+/PR+). In some embodiments, the breast cancer has spread to the lungs, bone, liver, and/or brain. In some embodiments, the breast cancer has spread to brain. In some embodiments, the HR+mBC (e.g., HR+HER2−mBC) is resistant to or has become resistant to treatment with other CDK 4/6 inhibitors (e.g., palbociclib, ribociclib and/or abemaciclib). In other embodiments, the HR+mBC (e.g., HR+HER2−mBC) is resistant to or has become resistant to treatment with endocrine therapy (e.g., anti-estrogen therapy). In some such embodiments, the HR+mBC (e.g., HR+HER2−mBC) is resistant to or has become resistant to one or more anti-estrogen therapies selected from selective estrogen receptor degraders (SERDs), selective estrogen receptor modulators (SERMs), estrogen receptor downregulators (ERDs), and aromatase inhibitors. In one such embodiment, the HR+mBC (e.g., HR+HER2−mBC) is resistant to fulvestrant. In still other embodiments, the HR+mBC is resistant to or has become resistant to treatment with the combination of another CDK 4/6 inhibitor (e.g., palbociclib, ribociclib and/or abemaciclib) and an endocrine therapy (e.g., fulvestrant).

In some embodiments, provided herein are methods of treating metastatic human epidermal growth factor receptor 2-positive breast cancer (HER2+mBC) in a subject in need thereof, comprising administering to the subject a compound of formula (I), or a pharmaceutically acceptable salt thereof. In some embodiments, the breast cancer has spread to the lungs, bone, liver, and/or brain. In some embodiments, the breast cancer has spread to brain. In some embodiments, the HER2+mBC is resistant to or has become resistant to treatment with other CDK 4/6 inhibitors (e.g., palbociclib, ribociclib and/or abemaciclib). In some embodiments, the HER2+mBC is resistant to or has become resistant to treatment with a HER2 targeted therapy (e.g., anti-HER2 antibody such as trastuzumab, pertuzumab, or margetuximab).

The compound of formula (I), or a pharmaceutically acceptable salt thereof, can be administered in combination with a HER2 targeted therapy when used for the treatment of HER2+mBC. Patients with HER2+mBC often develop high levels of metastases in secondary organs, particularly the brain. As set forth herein, the compound of formula (I) is highly effective in treating brain metastases owing to its ability to penetrate the blood-brain barrier. Therefore, treatment of HER2+mBC with a compound of formula (I), or a pharmaceutically acceptable salt thereof, and a HER2 targeted therapy can be particularly effective in eradicating or slowing the progression of metastases, particularly in the brain.

In particular embodiments, the compound of formula (I), or a pharmaceutically acceptable salt thereof is administered in combination with a HER2 targeted therapy to treat HER2+mBC. In some embodiments, the HER2 targeted therapy is anti-HER2 antibody. In some such embodiments, the anti-HER2 antibody is trastuzumab, pertuzumab or margetuximab. In some such embodiments, the anti-HER2 antibody is trastuzumab. In some embodiments, the HER2 targeted therapy is a small molecule inhibitor of HER2 such as tucatinib or lapatinib. In some embodiments, the small molecule inhibitor of HER2 is tucatinib. In some embodiments, the HER2 targeted therapy comprises trastuzumab and tucatinib or lapatinib. In some embodiments, the HER2 targeted therapy comprises trastuzumab and tucatinib. In other embodiments, the compound of formula (I), or a pharmaceutically acceptable salt thereof, is administered in combination with both a HER2 targeted therapy and an anti-estrogen therapy to treat HR+HER2+mBC. In particular embodiments, the HER2 targeted therapy is trastuzumab and the anti-estrogen therapy is fulvestrant. In other particular embodiments, the HER2 targeted therapy is tucatinib and the anti-estrogen therapy is fulvestrant.

In some embodiments, the compound of formula (I), or a pharmaceutically acceptable salt thereof, is administered in combination with an anti-metabolite therapy to treat HR+HER2+mBC. In some such embodiments, the anti-metabolite therapy is capecitabine. In other embodiments, the compound of formula (I) and the anti-metabolite therapy are administered in combination with an anti-HER2 antibody. For instance, in particular embodiments, the compound of formula (I), or a pharmaceutically acceptable salt thereof, is administered in combination with capecitabine and trastuzumab. In other embodiments, the compound of formula (I), or a pharmaceutically acceptable salt thereof, and the anti-metabolite therapy are administered in combination with a small molecule inhibitor of HER2. For instance, in particular embodiments, the compound of formula (I), or a pharmaceutically acceptable salt thereof is administered in combination with capecitabine and tucatinib. In other embodiments, the compound of formula (I), or a pharmaceutically acceptable salt thereof is administered in combination with trastuzumab, tucatinib and capecitabine.

In some embodiments, the compound of formula (I), or a pharmaceutically acceptable salt thereof, is administered in combination with an anti-estrogen therapy to treat HR+HER2− mBC or HR+HER2+mBC. In some such embodiments, the anti-estrogen therapy is a SERD, a SERM, an ERD, or an aromatase inhibitor. Particular anti-estrogen therapies that a be administered in combination with a compound of formula (I), or a pharmaceutically acceptable salt thereof, include, but are not limited to, fulvestrant, brilanestrant, elacestrant, amcenestrant, rintodestrant, giredestrant, AZD9833, LY3484356, elacestrant, ZN-c5, D-0502, SHR9549, tamoxifen, raloxifenee, toremifene, beopareststrol, anastrozole, letrozole, exemestane, vorozole, formestane, and fadrozole. In some embodiments, the compound of formula (I), or a pharmaceutically acceptable salt thereof, is administered in combination with an anti-estrogen therapy to treat HR+HER2−mBC or HR+HER2+mBC, the HR+HER2−mBC or HR+HER2+mBC is resistant to or has become resistant to treatment of the anti-estrogen therapy. In other embodiments, an anti-estrogen therapy had not previously been administered to the HR+HER2− mBC or HR+HER2+mBC patient prior to receiving the combination therapy.

Prostate Cancer

In another aspect, provided herein are methods of treating prostate cancer in a subject in need thereof, comprising administering to the subject a compound of formula (I), or a pharmaceutically acceptable salt thereof. In some embodiments, the method of treating prostate cancer comprises reduction of tumor volume. In some embodiments, the method of treating prostate cancer comprises tumor regression. In some embodiments, the prostate cancer is resistant to or has become resistant to treatment with other CDK 4/6 inhibitors (e.g., palbociclib, ribociclib and/or abemaciclib). In some embodiments, the prostate cancer is resistant to or has become resistant to treatment with an anti-androgen therapy. In some embodiments, provided herein are methods of treating castration-resistant prostate cancer (CRPC) in a subject in need thereof, comprising administering to the subject a compound of formula (I), or a pharmaceutically acceptable salt thereof. CRPC is characterized by continuous growth of the cancer, even when the amount of testosterone in the body is reduced to very low levels. The compound of formula (I), or a pharmaceutically acceptable salt thereof, can be administered by itself or with another anti-cancer agent. In some embodiments, the CRPC is resistant to or has become resistant to treatment with other CDK 4/6 inhibitors (e.g., palbociclib, ribociclib and/or abemaciclib). In other embodiments, the CPRC is resistant to or has become resistant to treatment with an anti-androgen therapy. In some embodiments, the prostate cancer (e.g., CPRC) is resistant to or has become resistant to treatment with an anti-androgen therapy and the anti-androgen therapy is bicalutamide, nilutamide, abiraterone acetate, enzalutamide, apalutamide, or darolutamide. In some embodiments, the anti-androgen therapy is abiraterone acetate. In some embodiments, the anti-androgen therapy is enzalutamide. In any of the preceding embodiments, the cancer has a mutation in the CDKN2A gene.

In another aspect, provided herein are methods of treating metastatic castration-resistant prostate cancer (mCRPC) in a subject in need thereof, comprising administering to the subject a compound of formula (I), or a pharmaceutically acceptable salt thereof. The compound of formula (I), or a pharmaceutically acceptable salt thereof, can be administered by itself or with another anti-cancer agent. In some embodiments, the mCRPC is resistant to or has become resistant to treatment with other CDK 4/6 inhibitors (e.g., palbociclib, ribociclib and/or abemaciclib). In other embodiments, the mCPRC is resistant to or has become resistant to treatment with an anti-androgen therapy.

In some embodiments, the compound of formula (I), or a pharmaceutically acceptable salt thereof, is administered to a prostate cancer patient in combination with at least one (e.g., one or two) anti-androgen therapy. In some embodiments, the compound of formula (I), or a pharmaceutically acceptable salt thereof, is administered to a CRPC (e.g., mCRPC) patient in combination with at least one (e.g., one or two) anti-androgen therapy. In some embodiments, the anti-androgen is a nonsteroidal antiandrogen. In some such embodiments, the nonsteroidal anti-androgen therapy is selected from bicalutamide, nilutamide, abiraterone acetate, enzalutamide, apalutamide, and darolutamide, or combinations thereof. In particular embodiments, the nonsteroidal anti-androgen therapy is enzalutamide. In other embodiments, the compound of formula (I), or a pharmaceutically acceptable salt thereof, can be administered to a CRPC (e.g., mCRPC) patient in combination with a PARP inhibitor such as olaparib or rucaparib. In still other embodiments, the compound of formula (I), or a pharmaceutically acceptable salt thereof, can be administered to a CRPC (e.g., mCRPC) patient in combination with a PARP inhibitor and an anti-androgen therapy (e.g., enzalutamide).

Other anti-cancer agents have shown some survival benefit for CRPC, including taxanes (e.g., docetaxel, paclitaxel and cabazitaxel), Sipuleucel T, Radium223, pembrolizumab, Lu177 PMSA therapy, platinum chemotherapy, and fosfesterol. However, similar to the anti-androgen therapies, CRPC often becomes progressively resistant to these anti-cancer agents. In accordance with the present disclosure, administration of the compound of formula (I), or a pharmaceutically acceptable salt thereof, can enhance or restore the effect of these various anti-cancer agents. Accordingly, the disclosure provides methods of treating CRPC (e.g., mCRPC) in a patient in need thereof, comprising administering to the patient a compound of formula (I), or a pharmaceutically acceptable salt thereof, wherein the CRPC is resistant to or as acquired resistance to one or more of an anti-androgen therapy a taxane therapy, Sipuleucel T, Radium223, pembrolizumab, Lu177 PMSA therapy, platinum chemotherapy, or fosfesterol. The disclosure further provides methods of treating CRPC (e.g., mCRPC) by administering a compound of formula (I), or a pharmaceutically acceptable salt thereof, in combination with one or more of a taxane (e.g., docetaxel, paclitaxel and cabazitaxel), Sipuleucel T, Radium223, pembrolizumab, Lu177 PMSA therapy, platinum chemotherapy, or fosfesterol. In some such embodiments, an anti-androgen therapy (e.g., enzalutamide) is also administered to the patient.

Brain Metastases

Surprisingly, it has been found in in vivo animal models, the compound of formula (I) shows very high brain concentrations following oral administration. See Example 6. The compound of formula (I) has a longer half-life in the brain than in the plasma. As a result of the high blood-brain-barrier penetration, a compound of the formula (I), or a pharmaceutically acceptable salt thereof, can be used to effectively treat various cancers of the brain or cancers from primary tumors that have metastasized to the brain.

Accordingly, in one aspect, the disclosure provides methods of treating brain metastases in a subject in need thereof, comprising administering to the subject a compound of formula (I), or a pharmaceutically acceptable salt thereof. Owing to its ability to effectively penetrate the blood-brain-barrier, the compound of formula (I) is capable of preventing the growth and survival of metastasizing cancer cells in the brain. The compound of formula (I), or a pharmaceutically acceptable salt thereof, can be used to treat metastases originating from any cancer. In some embodiments the primary cancer is a solid tumor. In some embodiments the primary cancer is any of adult and pediatric oncology, myxoid and round cell carcinoma, locally advanced tumors, metastatic cancer, human soft tissue sarcomas, including Ewing's sarcoma, cancer metastases, including lymphatic metastases, squamous cell carcinoma, particularly of the head and neck, esophageal squamous cell carcinoma, oral carcinoma, blood cell malignancies, including multiple myeloma, leukemias, including acute lymphocytic leukemia, acute nonlymphocytic leukemia, chronic lymphocytic leukemia, chronic myelocytic leukemia, and hairy cell leukemia, effusion lymphomas (body cavity based lymphomas), thymic lymphoma, cutaneous T cell lymphoma, Hodgkin's lymphoma, non-Hodgkin's lymphoma, cancer of the adrenal cortex, ACTH-producing tumors, lung cancer, including small cell carcinoma and nonsmall cell cancers, breast cancer, including small cell carcinoma and ductal carcinoma, gastrointestinal cancers, including stomach cancer, colon cancer, colorectal cancer, polyps associated with colorectal neoplasia, pancreatic cancer, liver cancer, urological cancers, including bladder cancer, including primary superficial bladder tumors, invasive transitional cell carcinoma of the bladder, and muscle-invasive bladder cancer, prostate cancer, malignancies of the female genital tract, including ovarian carcinoma, primary peritoneal epithelial neoplasms, cervical carcinoma, uterine endometrial cancers, vaginal cancer, cancer of the vulva, uterine cancer and solid tumors in the ovarian follicle, malignancies of the male genital tract, including testicular cancer and penile cancer, kidney cancer, including renal cell carcinoma, brain cancer, including intrinsic brain tumors, neuroblastoma, astrocytic brain tumors, gliomas, metastatic tumor cell invasion in the central nervous system, bone cancers, including osteomas and osteosarcomas, skin cancers, including melanoma, tumor progression of human skin keratinocytes, squamous cell cancer, thyroid cancer, retinoblastoma, neuroblastoma, peritoneal effusion, malignant pleural effusion, mesothelioma, Wilms's tumors, gall bladder cancer, trophoblastic neoplasms, hemangiopericytoma, and Kaposi's sarcoma. In particular embodiments, the primary cancer is breast cancer, prostate cancer, colon cancer, lung cancer, melanoma or leukemia. In some embodiments, the primary cancer is breast cancer, prostate cancer, colon cancer, lung cancer, melanoma, leukemia, renal cancer or hematopoietic cancer. In some embodiments, the cancer has a mutation in the CDKN2A gene.

In particular embodiments, the compound of formula (I), or a pharmaceutically acceptable salt thereof, is administered to a breast cancer patient, wherein the breast cancer has metastasized to the brain. It such embodiments, the breast cancer can be HR+mBC, HER2+mBC, or metastatic triple negative breast cancer. In some such embodiments where the breast cancer is HR+mBC, the breast cancer can be resistant to endocrine therapy, such as anti-estrogen therapy. In some such embodiments, the HR+HER2−mBC or HER2+mBC is resistant to one or more anti-estrogen therapies selected from SERDs, SERMs, ERDs, and aromatase inhibitors. In one such embodiment, the anti-estrogen therapy is fulvestrant. The compound of formula (I), or a pharmaceutically acceptable salt thereof, can be administered in combination with an anti-estrogen therapy to a breast cancer patient (e.g., a HR+HER2−mBC or HER2+mBC patient) in need thereof, wherein the cancer has metastasized to the brain. In some embodiments, compound of formula (I), or a pharmaceutically acceptable salt thereof, is administered in combination with fulvestrant, to a breast cancer patient (e.g., a HR+HER2−mBC or HER2+mBC patient) in need thereof, wherein the cancer has metastasized to the brain.

In embodiments where the HR+mBC (e.g., HR+HER2−mBC or HR+HER2+mBC) is resistant to prior anti-estrogen therapy, the compound of formula (I) can potentially restore the efficacy of the anti-estrogen therapy. For instance, in some embodiments, administration of the compound of formula (I), or a pharmaceutically acceptable salt thereof, can restore the efficacy of fulvestrant in HR+mBC (e.g., HR+HER2−mBC or HR+HER2+mBC) that is resistant to or has become resistant to fulvestrant.

In some embodiments, provided herein is a method of inhibiting CDK2, CDK4 and CDK6 in a metastasizing cancer cell in the brain comprising administering an effective amount of a compound of formula (I), or a pharmaceutically acceptable salt thereof. In some embodiments, CDK2, CDK4 or CDK6 are inhibited by about 10% or more, about 20% or more, about 30% or more, about 40% or more, about 50% or more, about 60% or more, about 70% or more, about 75% or more, about 80% or more, about 90% or more, about 95% or more, about 96% or more, about 97% or more, about 98% or more, or about 99% or more. In some such embodiments, CDK1 is not inhibited or is inhibited to a minimal extent. For instance, CDK1 can be inhibited about 30% or less, about 20% or less, about 10% or less, about 5% or less, or about 1% or less. In some embodiments, CDK2, CDK4 and/or CDK6 are inhibited up to about 99%, up to about 98%, up to about 97%, up to about 96%, up to about 95%, up to about 90%, up to about 85%, up to about 80%, up to about 70%, or up to about 60%. In some embodiments, the activity of CDK1, CDK2, CDK4 or CDK6 is measured according to a kinase assay.

In some embodiments, provided herein is a method of inhibiting the proliferation of a cancerous brain cell, comprising contacting the cell with an effective amount of a compound of formula (I), or a pharmaceutically acceptable salt thereof. In some embodiments, compound of formula (I), or a pharmaceutically acceptable salt thereof, is effective in inhibiting the proliferation of the cell with an EC50 of less than 5 μM, less than 2 μM, less than 1 μM, less than 900 nM, less than 800 nM, less than 700 nM, less than 600 nM, less than 500 nM, less than 400 nM, less than 300 nM, less than 200 nM, less than 100 nM, or less than 50 nM. In some embodiments, the compound of formula (I), or a pharmaceutically acceptable salt thereof is effective in inhibiting the proliferation of the cell with an EC50 between 10 nM and 20 nM, between 20 nM and 50 nM, between 50 nM and 100 nM, between 100 nM and 500 nM, between 500 nM and 1 μM, between 1 μM and 2 μM, or between 2 μM and 5 μM. In some embodiments, the EC50 is measured according to a cell proliferation assay.

Glioma

In some embodiments, provided is a method of treating methods of treating glioma in a subject in need thereof, comprising administering to the subject a compound of formula (I), or a pharmaceutically acceptable salt thereof. In some embodiments, the glioma is a high-grade glioma. In some embodiments, the method of treating glioma comprises reduction of tumor volume. In some embodiments, the method of treating glioma comprises tumor regression. In some embodiments, the method of treating glioma comprises prolonging survival. In any of the preceding embodiments, the cancer has a mutation in the CDKN2A gene.

High-grade gliomas are tumors of the glial cells, which are found in the brain and the spinal cord. Generally, high-grade gliomas fall into two categories, Grade III tumors (anaplastic astrocytoma, anaplastic oligodendroglioma and anaplastic ependymoma) and Grade IV tumors (glioblastoma). These high-grade gliomas may be further classified on whether they have a particular genetic change. For instance, some high-grade gliomas are characterized by a mutation in the isocitrate dehydrogenase (IDH) gene. Such IDH-mutant gliomas are often associated with low survival rates. One particular IDH-mutant involves homozygous deletion of the cyclin-dependent kinase inhibitor (CDKN2A) gene. Current treatment of high-grade gliomas involves surgery, radiation therapy and chemotherapeutic agents such as the alkylating agent temozolomide (TMZ). However, high-grade gliomas often develop resistance to TMZ.

In some embodiments, provided herein are methods of treating high-grade gliomas in a subject in need thereof, comprising administering to the subject a compound of formula (I), or a pharmaceutically acceptable salt thereof. The efficacy of the compound in formula (I) to treat high-grade gliomas stems, in part, from the ability of the compound to penetrate the blood-brain barrier. In one embodiment, the compound of formula (I), or a pharmaceutically acceptable salt thereof, is used to treat patients with Grade III gliomas such as anaplastic astrocytoma, anaplastic oligodendroglioma and anaplastic ependymoma. In one embodiment, the compound of formula (I), or a pharmaceutically acceptable salt thereof, is used to treat patients with Grade IV gliomas. In another embodiment, the compound of formula (I), or a pharmaceutically acceptable salt thereof, is used to treat patients with glioblastoma. In another embodiment, the compound of formula (I), or a pharmaceutically acceptable salt thereof, is used to treat patients with IDH-mutant gliomas. In some such embodiments, the high-grade glioma is characterized by a CDKN2A mutation. In still another embodiment, the compound of formula (I), or a pharmaceutically acceptable salt thereof, is used to treat high-grade gliomas without IDH mutations.

In some such embodiments, the compound of formula (I), or a pharmaceutically acceptable salt thereof, can be administered to high-grade glioma patient that is resistant to TMZ treatment. The compound of formula (I), or a pharmaceutically acceptable salt thereof, can be administered to a high-grade glioma patient that had previously been or is currently being treated with TMZ, regardless of whether the patient has developed resistance to TMZ. Therefore, the disclosure provides for methods of treating high-grade by administering a compound of formula (I), or a pharmaceutically acceptable salt thereof, in combination with TMZ. In one embodiment, the combination of the compound of formula (I), or a pharmaceutically acceptable salt thereof, and TMZ can be used to treat Grade III gliomas such as anaplastic astrocytoma, anaplastic oligodendroglioma and anaplastic ependymoma. In another embodiment, the combination of the compound of formula (I), or a pharmaceutically acceptable salt thereof, and TMZ can be used to treat glioblastoma.

### Pharmaceutical Compositions and Formulations

Pharmaceutical compositions of any of the compound of formula (I), or a pharmaceutically acceptable salt thereof, or a combination thereof as detailed herein are embraced by this invention. Thus, the invention includes pharmaceutical compositions comprising a compound of formula (I), or a pharmaceutically acceptable salt thereof, disclosed herein and a pharmaceutically acceptable carrier or excipient. In one embodiment, the pharmaceutical composition is a composition for controlled release of the compound of formula (I), or a pharmaceutically acceptable salt thereof.

In certain embodiments, the disclosed pharmaceutical compositions comprise a pharmaceutically acceptable salt of the compound of formula (I). In some such embodiments, the pharmaceutically acceptable salt of the compound of formula (I) is a hydrochloric acid (HCl) salt.

Compounds or compositions disclosed herein may be formulated for any available delivery route, including an oral, mucosal (e.g., nasal, sublingual, vaginal, buccal or rectal), parenteral (e.g., intramuscular, subcutaneous or intravenous), topical or transdermal delivery form, or a form suitable for inhalation. A compound or composition disclosed herein may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

Compounds disclosed herein can be used in the preparation of a formulation, such as a pharmaceutical formulation, by combining the compound of the formula (I), or a pharmaceutically acceptable salt thereof, as an active ingredient with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21^{st} ed. (2005), which is incorporated herein by reference.

In some embodiments, the pharmaceutical compositions described herein are in a unit dosage form suitable for single administration of precise dosages. In some instances, in unit dosage form, the formulation is divided into unit doses containing appropriate quantities of one or more compound. In certain embodiments, the unit dosage is in the form of a package containing discrete quantities of the formulation. Non-limiting examples are packaged tablets or capsules and powders in vials or ampoules. In some embodiments, aqueous suspension compositions are packaged in single-dose non-reclosable containers. In alternative embodiments, multiple-dose reclosable containers are used, in which case it is typical to include a preservative in the composition.

The compounds of formula (I), or pharmaceutically salts thereof, may be administered to a subject (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid poly-ols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

As set forth herein, other therapeutic agents, particularly anti-cancer agents, can be administered in combination with the compound of formula (I), or a pharmaceutically acceptable salt thereof. When the other additional therapeutic agent is administered in combination with the compound of formula (I), or a pharmaceutically acceptable salt thereof, the second therapeutic agent can be administered prior to, simultaneously with or after administration of the compound of formula (I), or a pharmaceutically acceptable salt thereof. In some embodiments, the compound of formula (I), or a pharmaceutically acceptable salt thereof, is combined in unitary dosage form with a second therapeutic agent. In one such embodiment, the compound of formula (I), or a pharmaceutically acceptable salt thereof and enzalutamide are combined in a unitary dosage for oral administration. In another embodiment, the compound of formula (I), or a pharmaceutically acceptable salt thereof and temozolomide are combined in a unitary dosage for oral administration.

### Dosing and Treatment Regimens

In certain embodiments, the compounds described herein are used in the preparation or manufacture of medicaments for advanced and metastasized cancers, as disclosed herein. In some embodiments, a method for treating any of the diseases or conditions described herein in a subject in need of such treatment, involves administration of pharmaceutical compositions containing a compound of formula (I), or a pharmaceutically acceptable salt, pharmaceutically acceptable salt thereof, in a therapeutically effective amount to said subject.

In certain embodiments, the compositions containing the compound of formula (I), or a pharmaceutically acceptable salt thereof, are administered for therapeutic treatments. In certain therapeutic applications, the compositions are administered to a patient already suffering from a disease or condition, in an amount sufficient to cure or at least partially arrest the symptoms of the disease or condition. In some embodiments, amounts effective for this use will depend on the severity and course of the disease or condition, previous therapy, the patient's health status, weight and response to the drugs and the judgment of the treating physician.

In certain embodiments, the amount of a given agent that corresponds to an effective amount varies depending upon factors such as the particular compound, disease or condition and its severity, the identity (e.g., weight) of the subject or host in need of treatment. In some embodiments, the effective amount is, nevertheless, determined according to the particular circumstances surrounding the case, including, e.g., the specific agent that is administered, the route of administration, the condition being treated and the subject or host being treated.

In certain embodiments, the compound of formula (I), or a pharmaceutically acceptable salt thereof, is administered orally to a subject to treat a cancer that has metastasized to the brain, a metastatic cancer such as HR+mBC, HER2+mBC, or mCRPC, or a high-grade glioma, as disclosed herein. In some embodiments, the compound of formula (I), or a pharmaceutically acceptable salt thereof, is administered to the subject once daily. In some embodiments, the subject is administered the compound of formula (I), or a pharmaceutically acceptable salt thereof, once daily on each day of the treatment period, without taking a dosing holiday. In these embodiments, an indicated daily dose is in the range from about 10 mg to about 250 mg, administered in a single dosage form or in divided dosages including, but not limited to, up to four times a day or in extended release form. The dosage amount of a compound as described herein is determined based on the free base of the compound of formula (I). In certain embodiments, suitable unit dosage forms for oral administration comprise from about 10 to about 250 mg active ingredient. In other embodiments, suitable unit dosage forms for oral administration comprise from about 20 to about 150 mg active ingredient. In other embodiments, suitable unit dosage forms for oral administration comprise from about 25 to about 125 mg active ingredient. In other embodiments, suitable unit dosage forms for oral administration comprise from about 25 to about 100 mg active ingredient. In other embodiments, suitable unit dosage forms for oral administration comprise from about 50 to about 100 mg active ingredient. In other embodiments, suitable unit dosage forms for oral administration comprise about 50 mg of active ingredient. In other embodiments, suitable unit dosage forms for oral administration comprise about 75 mg of active ingredient. In other embodiments, suitable unit dosage forms for oral administration comprise about 100 mg active ingredient. In other embodiments, suitable unit dosage forms for oral administration comprise about 125 mg active ingredient. In other embodiments, suitable unit dosage forms for oral administration comprise about 150 mg active ingredient. In other embodiments, suitable unit dosage forms for oral administration comprise about 200 mg active ingredient. In other embodiments, suitable unit dosage forms for oral administration comprise about 250 mg active ingredient.

In certain embodiments, the compound of formula (I), or a pharmaceutically acceptable salt thereof, is administered orally once daily to a subject to treat a cancer that has metastasized to the brain, such as HR+mBC, HER2+mBC, or mCRPC, or a high-grade glioma, as disclosed herein. In some embodiments, the subject is administered the compound of formula (I), or a pharmaceutically acceptable salt thereof, once daily on each day of the treatment period, without taking a dosing holiday. In these embodiments, an indicated daily is in the range from about 10 mg to about 250 mg, administered in a single dosage form or in divided dosages including, but not limited to, up to four times a day or in extended release form. In some such embodiments, once daily oral administration comprises from about 10 to about 200 mg active ingredient. In other embodiments, once daily oral administration comprises from about 20 to about 150 mg active ingredient. In other embodiments, once daily oral administration comprises from about 25 to about 125 mg active ingredient. In other embodiments, once daily oral administration comprise from about 25 to about 100 mg active ingredient. In other embodiments, once daily oral administration comprise from about 50 to about 100 mg active ingredient. In other embodiments, once daily oral administration comprises about 50 mg of active ingredient. In other embodiments, once daily oral administration comprises about 75 mg of active ingredient. In other embodiments, once daily oral administration comprises about 100 mg of active ingredient. In other embodiments, once daily oral administration comprises about 125 mg of active ingredient. In other embodiments, once daily oral administration comprises about 150 mg of active ingredient. In other embodiments, once daily oral administration comprises about 200 mg of active ingredient. In other embodiments, once daily oral administration comprises about 250 mg of active ingredient. In certain embodiments, the compound of formula (I), or a pharmaceutically acceptable salt thereof, is administered orally less frequently (e.g., every other day) to a subject to treat a cancer that has metastasized to the brain, such as HR+mBC, HER2+mBC, or mCRPC, or a high-grade glioma, as disclosed herein.

When administered in combination with additional therapeutic agents, as disclosed herein, the second therapeutic agent can be administered at doses that are typically administered when the second agent is administered alone. Alternatively, as a result of the synergy observed with the combination, the second therapeutic agent can be administered at doses that are lower (i.e., sub-therapeutic doses) than doses when either agent is administered alone.

In certain embodiments where the compound of formula (I), or a pharmaceutically acceptable salt thereof, is administered in combination with fulvestrant for the treatment of HR+HER2−mBC, HR+HER2+mBC, the compound of formula (I) can be dosed once daily in amounts described above and fulvestrant can be dosed as reflected on the label of FASLODEX (see https://dailymed.nlm.nih.gov/dailymed/fda/fdaDrugXsl.cfm?setid=83d7a440-e904-4e36-afb5-cb02b1c919f7&type=display which is hereby incorporated by reference). In other embodiments where the compound of formula (I), or a pharmaceutically acceptable salt thereof, is administered in combination with fulvestrant for the treatment of HR+HER2−mBC, HR+HER2+mBC, the compound of formula (I) can be dosed once daily in amounts described above and fulvestrant can be dosed intramuscularly once monthly at a dose from about 100 mg to about 500 mg. For instance, fulvestrant can be dosed intramuscularly once or twice monthly at a dose of about 100 mg, 200 mg, 300 mg, 400 mg, or 500 mg. For instance, fulvestrant can be dosed intramuscularly once monthly at a dose of about 250 mg.

In certain embodiments where the compound of formula (I), or a pharmaceutically acceptable salt thereof, is administered in combination with trastuzumab for the treatment of HR+HER2−mBC, HR+HER2+mBC, the compound of formula (I) can be dosed once daily in amounts described above and trastuzumab can be dosed as reflected on the label of HERCEPTIN (see https://dailymed.nlm.nih.gov/dailymed/fda/fdaDrugXsl.cfm?setid=492dbdb2-077e-4064-bff3-372d6af0a7a2&type=display and https://www.herceptin.com/hcp/dosing-admin.html, each of which is hereby incorporated by reference). In other embodiments where the compound of formula (I), or a pharmaceutically acceptable salt thereof, is administered in combination with trastuzumab for the treatment of HR+HER2−mBC, HR+HER2+mBC, the compound of formula (I) can be dosed once daily in amounts described above and trastuzumab can be dosed intravenously once weekly or once every three weeks at a dose of between about 2 mg/kg and about 8 mg/kg. For instance, trastuzumab can be dosed intravenously once weekly or once every three weeks at a dose of about 2 mg/kg, about 3 mg/kg, about 4 mg/kg, about 5 mg/kg, about 6 mg/kg, about 7 mg/kg, or about 8 mg/kg. In one example, trastuzumab can be dosed intravenously at an initial dose of 4 mg/kg, followed by dosing once weekly at a dose of about 2 mg/kg. In another example, trastuzumab can be dosed intravenously at an initial dose of 4 mg/kg, followed by dosing once weekly for a first time period at a dose of about 2 mg/kg, followed by dosing once every three weeks for a second time period at a dose of about 6 mg/kg. In another example, trastuzumab can be dosed intravenously at an initial dose of 8 mg/kg, followed by dosing once every three weeks at a dose of about 6 mg/kg.

In certain embodiments where the compound of formula (I), or a pharmaceutically acceptable salt thereof, is administered in combination with trastuzumab for the treatment of HR+HER2−mBC, HR+HER2+mBC, the compound of formula (I) can be dosed once daily in amounts described above and trastuzumab can be dosed as reflected on the label of HERCEPTIN HYLECTA (see https://dailymed.nlm.nih.gov/dailymed/fdagdaDrugXsl.cfm?setid=ebf30894-41cf-480c-8bc3-56f592a13813&type=display, which is hereby incorporated by reference). In other embodiments where the compound of formula (I), or a pharmaceutically acceptable salt thereof, is administered in combination with trastuzumab for the treatment of HR+HER2−mBC, HR+HER2+mBC, the compound of formula (I) can be dosed once daily in amounts described above and trastuzumab can be dosed subcutaneously once every three weeks at a dose of about 600 mg trastuzumab.

In certain embodiments where the compound of formula (I), or a pharmaceutically acceptable salt thereof, is administered in combination with enzalutamide for the treatment of mCRPC, the compound of formula (I) can be dosed once daily in amounts described above and enzalutamide can be dosed as reflected on the label of XTANDI®. In certain embodiments where the compound of formula (I), or a pharmaceutically acceptable salt thereof, is administered in combination with enzalutamide for the treatment of mCRPC, the compound of formula (I) can be dosed once daily in amounts described above and enzalutamide can be dosed once orally at a dose of from about 40 mg to about 240 mg, from about 40 mg to about 160 mg, from about 80 mg to about 160 mg, from about 100 mg to about 160 mg, or from about 80 mg to about 120 mg. For instance, enzalutamide can be dosed intramuscularly once monthly at a dose of about 40 mg, 50 mg, 80 mg, 100 mg, 120 mg, 130 mg, 140 mg, 150 mg, or 160 mg. In some embodiments, the compound of formula (I), or a pharmaceutically acceptable salt thereof, and enzalutamide are provided in a single unit dosage form for oral administration.

### Kits

The present disclosure further provides kits for carrying out the methods of the invention, which comprises a compound of formula (I), or a pharmaceutically acceptable salt thereof, or a composition comprising a compound of formula (I), or a pharmaceutically acceptable salt thereof. The kits may be used for any one or more of the uses described herein, and, accordingly, may contain instructions for the treatment of cancer.

Kits generally comprise suitable packaging. The kits may comprise one or more containers comprising any compounds described herein. Each component (if there is more than one component) can be packaged in separate containers or some components can be combined in one container where cross-reactivity and shelf life permit.

The kits may be in unit dosage forms, bulk packages (e.g., multi-dose packages) or sub-unit doses. For example, kits may be provided that contain sufficient dosages of a compound of formula (I), or a pharmaceutically acceptable salt thereof, as disclosed herein and/or a second pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present invention. The instructions included with the kit generally include information as to the components and their administration to an individual.

The invention can be further understood by reference to the following examples, which are provided by way of illustration and are not meant to be limiting.

### EXAMPLES

The following examples are provided to further aid in understanding the embodiments disclosed in the application, and presuppose an understanding of conventional methods well known to those persons having ordinary skill in the art to which the examples pertain. The particular materials and conditions described hereunder are intended to exemplify particular aspects of embodiments disclosed herein and should not be construed to limit the reasonable scope thereof.

### Example S1: Synthesis of the Compound of Formula (I) and the Hydrochloric Acid Salt Thereof

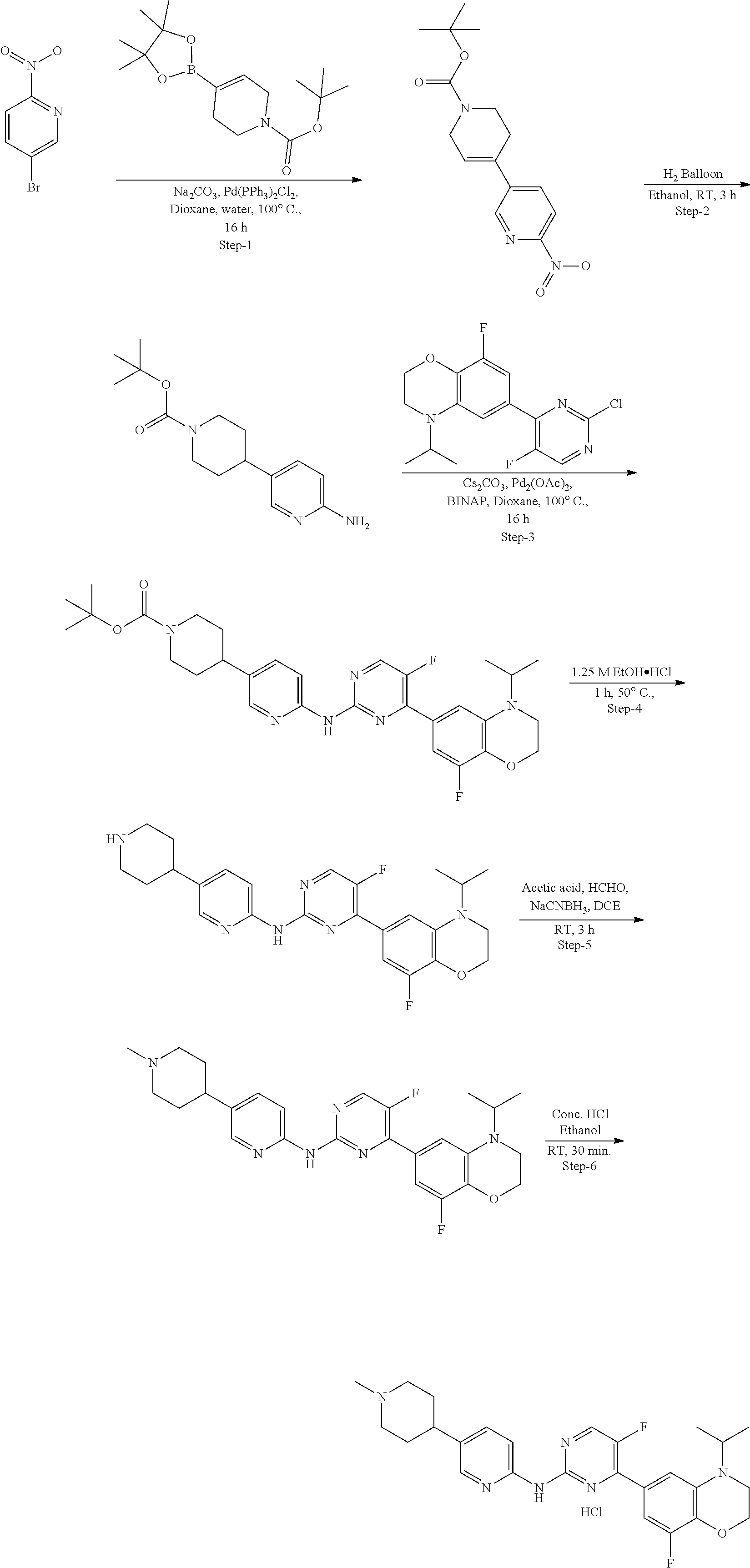

### Step-1: Synthesis of tert-butyl 6-nitro-3′,6′-dihydro-[3,4′-bipyridine]-1′(2′H)-carboxylate

To a solution of 5-bromo-2-nitropyridine (10 g, 49 mmol, 1 equiv) in dioxane (90 mL) and water (10 mL), was added tert-butyl 4-(4, 4, 5, 5-tetramethyl-1, 3, 2-dioxaborolan-2-yl)-3, 6-dihydropyridine-1(2H)-carboxylate (15.23 g, 49 mmol, 1 equiv). Sodium carbonate (15.58 g, 147 mmol, 3 eq.) was added to reaction mixture at ambient temperature and nitrogen was purged for 15 minutes. Pd(PPh₃)₂Cl₂ (343 mg, 0.49 mmol, 1 mol %) was added and nitrogen was again purged for 10 minutes. Reaction mixture was heated at 100° C. for 16 h. TLC (50% ethyl acetate:hexane) showed that starting material was consumed. After completion of reaction, solvent was removed under reduced pressure. Ethyl acetate (1000 mL) was added to reaction mixture and organic phase was separated. Ethyl acetate layer was washed with water (200 mL×3), dried over anhydrous sodium sulphate, filtered and concentrated under reduced pressure to afford crude. Crude product was purified by Combi-Flash to afford tert-butyl 6-nitro-3′,6′-dihydro-[3,4′-bipyridine]-1′(2′H)-carboxylate (10.2 g, 67.5%) as white solid. LCMS: 276 [M+H]⁺

### Step-2: Synthesis of tert-butyl tert-butyl 4-(6-aminopyridin-3-yl)piperidine-1-carboxylate

To a stirred solution of tert-butyl 6-nitro-3′,6′-dihydro-[3,4′-bipyridine]-1′(2′H)-carboxylate (10.2 g, 33.4 mmol, 1 equiv) in ethanol (400 mL), was added Pd/C (10% w/w, 2 g). The resultant reaction mixture was stir at ambient temperature for 2h under hydrogen balloon. TLC (50% EA:hexane) showed that starting material was consumed. After completion of the reaction, the mixture was passes through celite bed which was washed with ethanol (100 mL×2). Filtrate was concentrated under reduced pressure to afford tert-butyl tert-butyl 4-(6-aminopyridin-3-yl)piperidine-1-carboxylate (10 g, >100%) as a transparent oil. LCMS: 278 [M+H]⁺

### Step-3: Synthesis of tert-butyl 4-(6-((5-fluoro-4-(8-fluoro-4-isopropyl-3, 4-dihydro-2H-benzo[b][1,4]oxazin-6-yl)pyrimidin-2-yl)amino)pyridin-3-yl)piperidine-1-carboxylate

To a solution of 6-(2-chloro-5-fluoropyrimidin-4-yl)-8-fluoro-4-isopropyl-3,4-dihydro-2H-benzo[b][1,4]oxazine (10.6 g, 32 mmol, 1 equiv) in dioxane (160 mL), was added tert-butyl 4-(5-aminopyridin-2-yl) piperidine-1-carboxylate (10 g, 36 mmol, 1.1 equiv) and cesium carbonate (20.8 g, 64 mmol, 2 equiv) and nitrogen was purged for 15 minutes. Palladium acetate (2 mg, 0.009 mmol, 0.02 equiv) and BINAP (12 mg, 0.018 mmol, 0.04 equiv) were added and nitrogen was again purged for 10 minutes. Reaction mixture was heated at 100° C. for 16 h. TLC (50% ethyl acetate:hexane) showed that starting material was consumed. After completion of reaction, solvent was removed under reduced pressure. Ethyl acetate (1000 mL) was added to reaction mixture and organic phase was separated. Ethyl acetate layer was washed with water (200 mL×3), dried over anhydrous sodium sulphate, filtered and concentrated under reduced pressure to afford crude. Crude product was purified by Combi-Flash using 0-40% ethyl acetate:Hexane to afford tert-butyl 4-(6-((5-fluoro-4-(8-fluoro-4-isopropyl-3, 4-dihydro-2H-benzo[b][1,4]oxazin-6-yl)pyrimidin-2-yl)amino)pyridin-3-yl)piperidine-1-carboxylate (7 g, 38%) as a yellow solid compound. LCMS: 567 [M+H]⁺

### Step-4: Synthesis of 5-fluoro-4-(8-fluoro-4-isopropyl-3,4-dihydro-2H-benzo[b][1,4]oxazin-6-yl)-N-(5-(piperidin-4-yl)pyridin-2-yl)pyrimidin-2-amine hydrochloride

A solution of tert-butyl tert-butyl 4-(6-((5-fluoro-4-(8-fluoro-4-isopropyl-3,4-dihydro-2H-benzo[b][1,4]oxazin-6-yl)pyrimidin-2-yl)amino)pyridin-3-yl)piperidine-1-carboxylate (7 g, 0.1 mmol, 1 equiv) was charged in ethanol (60 mL) and 4 M HCl in Dioxane (40 mL) was added into it. Solution was stirred for 1 h at 50° C. TLC (50% ethyl acetate:hexane) and LCMS showed that starting material was consumed. After completion of the reaction, solvent was removed under reduced pressure and basified with saturated NaHCO₃ (˜100 mL) till pH=7-8. Solid obtained was filtered under vacuum and washed with water (100 mL×3). Compound was further dried in vacuum to afford 5-fluoro-4-(8-fluoro-4-isopropyl-3,4-dihydro-2H-benzo[b][1,4]oxazin-6-yl)-N-(5-(piperidin-4-yl)pyridin-2-yl)pyrimidin-2-amine hydrochloride (5.2 g, 90.2%) as a yellow solid. LCMS: 467 [M+H]

### Step-5: Synthesis of 5-fluoro-4-(8-fluoro-4-isopropyl-3,4-dihydro-2H-benzo[b][1,4]oxazin-6-yl)-N-(5-(1-methylpiperidin-4-yl)pyridin-2-yl)pyrimidin-2-amine

To a stirred solution of 5-fluoro-4-(8-fluoro-4-isopropyl-3, 4-dihydro-2H-benzo[b][1,4]oxazin-6-yl)-N-(6-(piperidin-4-yl)pyridin-3-yl)pyrimidin-2-amine (4 g, 8.56 mmol, 1 equiv) in DCE (40 mL), was added Formaldehyde (40% in water) (2.31 g, 77.04 mmol, 9 equiv), acetic acid (2.57 g, 42.8 mmol, 5 equiv). The reaction mixture was stirred at ambient temperature for 1 h. The reaction mixture was cooled to 0° C. NaCNBH₃ (1.61 g, 25.68 mmol, 3 equiv) was added to above mixture and reaction mixture was allowed to come at ambient temperature. The reaction mixture was stirred at ambient temperature for 2h. TLC (10% MeOH:DCM) and LCMS showed that starting material was consumed. After completion, the reaction mixture was diluted with water (50 mL) and concentrated under reduced pressure. Saturated bicarbonate solution (100 mL) was added in to crude material and solid obtained was filtered under vacuum. Crude material was purified by silica gel chromatography (#100-200) using 0-7% MeOH:DCM to afford free base of 5-fluoro-4-(8-fluoro-4-isopropyl-3,4-dihydro-2H-benzo[b][1,4]oxazin-6-yl)-N-(5-(1-methylpiperidin-4-yl)pyridin-2-yl)pyrimidin-2-amine (1.42 g, 34.5%) as a yellow solid. LCMS: 481 [M+H]⁺

### Step-6: Synthesis of 5-fluoro-4-(8-fluoro-4-isopropyl-3,4-dihydro-2H-benzo[b][1,4]oxazin-6-yl)-N-(5-(1-methylpiperidin-4-yl)pyridin-2-yl)pyrimidin-2-amine hydrochloride

In 250 mL RBF, 5-fluoro-4-(8-fluoro-4-isopropyl-3,4-dihydro-2H-benzo[b][1,4]oxazin-6-yl)-N-(5-(1-methylpiperidin-4-yl)pyridin-2-yl)pyrimidin-2-amine free base (1.41 g, 2.93 mmol, 1 eq.) was suspended in ethanol (100 mL) and heated to reflux till the suspension became clear solution. Reaction mixture was cooled to ambient temperature. Hydrochloric acid, 35% (611 mg, 5.86 mmol, 2 eq.) dissolved in ethanol (10 mL) was added to reaction mixture drop wise at ambient temperature. Reaction mixture was stirred at same temperature for 30 minutes. Reaction mixture was concentrated under reduced pressure. MTBE (100 mL) was added to reaction mixture and solid obtained was filtered under vacuum. Solid compound was washed with methyl tert butyl ether (100 mL) and dried in vacuum oven at 55° C. for 16h to afford 5-fluoro-4-(8-fluoro-4-isopropyl-3,4-dihydro-2H-benzo[b][1,4]oxazin-6-yl)-N-(5-(1-methylpiperidin-4-yl)pyridin-2-yl)pyrimidin-2-amine hydrochloride salt of tittle compound (1.57 g, 96.9%) as yellow solid. LCMS: 481 [M+1]⁺; ¹H NMR (400 MHz, DMSO-d6, HCl salt): δ 10.61 (br. s., 1H), 8.75 (d, J=3.51 Hz, 1H), 8.24 (s, 1H), 8.01 (s, 1H), 7.92 (d, J=8.77 Hz, 1H), 7.44 (s, 1H), 7.22 (d, J=11.40 Hz, 1H), 4.31 (s, 2H), 4.06-4.23 (m, 1H), 3.50 (d, J=12.28 Hz, 2H), 3.32 (m, 2H), 3.06 (m, 1H), 2.90 (m, 3H), 2.77 (m, 2H), 2.03 (m, 2H), 1.97 (m, 2H), 1.20 (d, J=6.58 Hz, 6H); UPLC-LCMS: 99.93%; HPLC: 99.05%.

### Example 1: Evaluation of the Compound of Formula (I) in a MCF-7 Breast Tumor Model—Monotherapy or Combination with Fulvestrant

1.1 Study

The in vivo anti-tumor efficacy of the compound of formula (I) either alone or in combination with fulvestrant on MCF-7 breast tumor model on female Balb/c nude mice was assessed. MCF-7 cells are a breast cancer cell line that is HR+HER2−. See Booms et al., (2019) Cancer Epidemiol Biomarkers Prev 28(10); 1735-1745, which is hereby incorporated by reference.

The study was in two parts. The first part was designed to measure tumor growth inhibition (TGI). The second part was used to measure pharmacokinetic and pharmacodynamic (PKPD) parameters. In the studies, the compound of formula (I) was dosed orally (po) and fulvestrant was dosed subcutaneously.

1.2 Experimental Design

In the study to measure TGI, the compound of formula (I) was dosed orally (po) once daily (QD) and fulvestrant (when administered) was dosed subcutaneously (sc) once weakly (QW) for 49 days, as shown in Table 1. For the PKPD study, single doses of the compound of formula (I) either alone or together with fulvestrant were administered as described in Table 2.

TABLE 1
Study design for TGI part

| Group | n | Treatment | Dose | | Dosing Volume | | Dosing Route | Schedule |
|---|---|---|---|---|---|---|---|---|
| 1 | 8 | Vehicle | — | | 10 | μl/g | PO | QD*37 days |
| 2 | 8 | Fulvestrant | 3 | mg/dose | 60 | μl/mouse | SC | QW*49 days |
| 3 | 8 | Compound | 10 | mg/kg | 10 | μl/g | PO | QD*49 days |
| | | of formula | | | | | | |
| | | (I) | | | | | | |
| 4 | 8 | Compound | 30 | mg/kg | 10 | μl/g | PO | QD*49 days |
| | | of formula | | | | | | |
| | | (I) | | | | | | |
| 5 | 8 | Fulvestrant + | 3 mg/dose + | | 60 μl/mouse + | | SC + PO | QW + |
| | | Compound | 10 mg/kg | | 10 μl/g | | | QD*49 days |
| | | of Formula | | | | | | |
| | | (I) | | | | | | |
| 6 | 8 | Fulvestrant + | 3 mg/dose + | | 60 μl/mouse + | | SC + PO | QW + |
| | | Compound | 30 mg/kg | | 10 μl/g | | | QD*49 days |
| | | of formula | | | | | | |
| | | (I) | | | | | | |
| 7 | 8 | Fulvestrant | 1 | mg/dose | 20 | μl/mouse | SC | QW*49 days |

| | | | | | | |
|---|---|---|---|---|---|---|
| 8 | 8 | Fulvestrant + | 1 mg/dose + | 20 μl/mouse + | SC + PO | QW + |
| | | Compound | 10 mg/kg | 10 μl/g | | QD*49 days |
| | | of formula | | | | |
| | | (I) | | | | |
| 9 | 8 | Fulvestrant + | 1 mg/dose + | 20 μl/mouse + | SC + PO | QW + |
| | | Compound | 30 mg/kg | 10 μl/g | | QD*49 days |
| | | of formula | | | | |
| | | (I) | | | | |

TABLE 2
Study design for PKPD part

| Group | n | Treatment | Dose | | Dosing Volume | | Dosing Route | Schedule |
|---|---|---|---|---|---|---|---|---|
| 1 | 3 | Vehicle | — | | 10 | μl/g | PO | Single dose |
| 2 | 3 | Fulvestrant | 3 | mg/dose | 60 | μl/mouse | SC | Single dose |
| 3 | 3 | Compound | 10 | mg/kg | 10 | μl/g | PO | Single dose |
| | | of formula | | | | | | |
| | | (I) | | | | | | |
| 4 | 3 | Compound | 30 | mg/kg | 10 | μl/g | PO | Single dose |
| | | of formula | | | | | | |
| | | (I) | | | | | | |
| 5 | 3 | Fulvestrant + | 3 mg/dose + | | 60 μl/mouse + | | SC + PO | Single dose |
| | | Compound | 10 mg/kg | | 10 μl/g | | | |
| | | of formula | | | | | | |
| | | (I) | | | | | | |
| 6 | 3 | Fulvestrant + | 3 mg/dose + | | 60 μl/mouse + | | SC + PO | Single dose |
| | | Compound | 30 mg/kg | | 10 μl/g | | | |
| | | of formula | | | | | | |
| | | (I) | | | | | | |
| 7 | 3 | Fulvestrant | 1 | mg/dose | 20 | μl/mouse | SC | Single dose |

| | | | | | | |
|---|---|---|---|---|---|---|
| 8 | 3 | Fulvestrant + | 1 mg/dose + | 20 μl/mouse + | SC + PO | Single dose |
| | | Compound | 10 mg/kg | 10 μl/g | | |
| | | of formula | | | | |
| | | (I) | | | | |
| 9 | 3 | Fulvestrant + | 1 mg/dose + | 20 μl/mouse + | SC + PO | Single dose |
| | | Compound | 30 mg/kg | 10 μl/g | | |
| | | of formula | | | | |
| | | (I) | | | | |

1.3 Materials

1.3.1 Animals

Female Balb/c nude mice with the following characteristics were used in the study:
Species: Mus musculus
Strain: Balb/c nude
Age: 6-8 weeks
Sex: Female
Body weight: 18-22 g
Number of animals: 108 mice
Animal supplier: Beijing Vital River Laboratory Animal Co., LTD.

1.3.2 Housing Conditions

The mice were kept in individual ventilation cages at constant temperature and humidity.
Temperature: 20-26° C.
Humidity: 40-70%.
Cages: Made of polycarbonate. The size is 300 mm×180 mm×150 mm. The bedding material is corn cob, which is changed twice per week.
Diet: Animals had free access to irradiation sterilized dry granule food during the entire study period.
Water: Animals had free access to sterile drinking water.
Cage identification: The identification labels for each cage contained the following information: number of animals, sex, strain, data received, treatment, study number, group number and the starting date of the treatment.
Animal identification: Animals were marked by ear coding.

1.4 Experimental Methods and Procedures

1.4.1 Cell Culture

The MCF-7 tumor cells were maintained in vitro in MEM supplemented with 10% heat inactivated fetal bovine serum at 37° C. in an atmosphere of 5% CO₂ in air. The tumor cells were routinely subcultured twice weekly. The cells growing in an exponential growth phase were harvested and counted for tumor inoculation.

1.4.2 Tumor Inoculation and Group Assignment

Each mouse was inoculated subcutaneously at the right flank with MCF-7 (10*10⁶) cells in 0.2 mL of PBS mixed with Matrigel (50:50) for tumor development. 17β-Estradiol (0.36 mg, 60-day release) pellets (Innovative Research of America Cat. No.: SE-121, pellet size: 3.0 mm) were implanted 2 days before cell inoculation. The mice need help to urination from day 7 after 17β-Estradiol pellets implantation.

In TGI part, the treatments was started on day 7 after cell inoculation when the average tumor volume reaches 173 mm³. 72 mice were selected and assigned into 9 groups. The testing articles were administrated to the mice according to Table 1.

In PKPD part, the treatments was started on day 21 after cell inoculation when the average tumor volume reaches 569 mm³. 27 mice were selected and assigned into 9 groups. The testing articles were administrated to the mice according to Table 2.

1.4.3 Testing Articles Formulation Preparation

Formulations for administration were prepared as shown in Table 3.

TABLE 3
Formulation preparation

| Compounds | Preparation | Concentration | | Storage |
|---|---|---|---|---|
| Vehicle | 0.5% HPMC + 0.2% Tween | — | | 4° C. |
| Compound | Suspend 41.25 mg of the | 1 | mg/mL | 4° C. |
| (I) | compound of formula (I) | | | |
| | in 41.044 mL vehicle, | | | |
| | vortex and stir to obtain | | | |
| | a white uniform suspension. | | | |
| Compound | Suspend 125.66 mg of the | 3 | mg/mL | 4° C. |
| of formula | compound of formula | | | |
| (I) | (I) in 41.677 mL vehicle, | | | |
| | vortex and stir to obtain | | | |
| | a white uniform suspension. | | | |
| Fulvestrant | Ready to use | 50 | mg/ml | 4° C. |
| injection | | | | |

1.5 Observations

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). At the time of routine monitoring, the animals were daily checked for any effects of tumor growth and treatments on normal behavior such as mobility, food and water consumption, body weight gain/loss, eye and any other abnormal effect as stated in the protocol. Death and observed clinical signs were recorded on the basis of the numbers of animals within each subset. Animals that were observed to be in a continuing deteriorating condition or their tumor size exceeding 3000 mm³ were euthanized prior to death or before reaching a comatose state.

1.5.1 Tumor Measurements and the Endpoints

For TGI part, the major endpoint was to see if the tumor growth could be delayed or mice could be cured. Tumor size was measured twice weekly in two dimensions using a caliper, and the volume was expressed in mm³ using the formula: V=0.5 a×b² where a and b are the long and short diameters of the tumor, respectively. The tumor sizes are then used for the calculation of T/C and TGI values.

The T/C value (in percent) is an indication of antitumor effectiveness; T and C are the mean volume of the treated and control groups, respectively, on a given day.

TGI was calculated for each group using the formula: TGI (%)=[1−(Tᵢ−T₀)/(Vᵢ−V₀)]×100; Tᵢ is the average tumor volume of a treatment group on PG-Di, T₀ is the average tumor volume of the treatment group on the day of treatment start, Vᵢ is the average tumor volume of the vehicle control group on the same day with Tᵢ, and V₀ is the average tumor volume of the vehicle group on the day of treatment start.

Vehicle group in TGI study was taken down on PG-D37 because of the average tumor size reached 2005 mm³, the other groups were terminated on PG-D49.

For PKPD part, the mice were euthanized at pre-determined time points for sample collection.

1.5.2 Statistical Analysis

Summary statistics, including mean and the standard error of the mean (SEM), are provided for the tumor volume of each group at each time point.

For comparison among of tumor volume of groups 2-9 and group 1, a one-way ANOVA was performed. All the analysis were conducted using GraphPad Prism software. p<0.05 was considered to be statistically significant.

1.5.3 Sample Collection

For TGI part, tumor was collected from vehicle group at 6h and 24h on day 37. Plasma was collected from groups 2-9 at 1, 2, 4, 6, 8 and 24h on day 49. Tumors were collected from these groups at 6 and 24 h after last dose on day 49, each tumor was cut into 3 parts, one for PK, one for PD, one as backup.

For PKPD part, plasma was collected from groups 2-9 at 1, 2, 4, 6, 8 and 24h, tumor was collected from all groups at 24h. Each tumor was cut into 3 parts, one for PK, one for PD, one for FFPE preparation.

1.6 Results

1.6.1 Body Weight Gain or Loss

Animal body weight was monitored regularly as an indirect measurement of toxicity. One mouse in groups 3, 4 and two mice in group 9 were euthanized for more than 20% body weight loss caused by urinary system abnormal, one mouse in group 8 was suspended treatment on PG-D42 for over 15% body weight loss. All other animals kept the body weight well, indicating that the compound of formula (I) was not toxic to the animals being studied.

1.6.2 Tumor Volume Measurement

Mean tumor volume over time of female Balb/c nude mice bearing MCF-7 tumors are shown in Table 4.

TABLE 4
Tumor volume measurement (mm³, mean ± SEM)

| | | | | | Group 5 |
|---|---|---|---|---|---|
| | | | | | Fulvestrant + |
| | Group 1 | Group 2 | Group 3 | Group 4 | Formula (I) |
| | Vehicle | Fulvestrant | Formula (I) | (Formula (I) | 3 mg/dose + |
| | — | 3 mg/dose | 10 mg/kg | 30 mg/kg | 10 mg/kg |
| Days | QD | QW | QD | QD | QW + QD |
| 0 | 173 ± 10 | 173 ± 13 | 173 ± 12 | 173 ± 11 | 173 ± 11 |
| 3 | 207 ± 10 | 181 ± 9 | 195 ± 14 | 192 ± 16 | 183 ± 15 |
| 6 | 287 ± 28 | 205 ± 16 | 188 ± 25 | 151 ± 24 | 211 ± 21 |
| 9 | 367 ± 29 | 240 ± 12 | 185 ± 15 | 153 ± 19 | 217 ± 30 |
| 13 | 518 ± 40 | 310 ± 25 | 179 ± 21 | 147 ± 17 | 221 ± 31 |
| 16 | 668 ± 62 | 337 ± 33 | 187 ± 22 | 154 ± 18 | 232 ± 31 |
| 20 | 856 ± 85 | 285 ± 32 | 228 ± 36 | 159 ± 21 | 196 ± 35 |
| 23 | 1017 ± 111 | 288 ± 44 | 268 ± 37 | 175 ± 23 | 182 ± 33 |
| 27 | 1240 ± 129 | 275 ± 35 | 310 ± 56 | 168 ± 25 | 183 ± 38 |
| 30 | 1469 ± 175 | 294 ± 35 | 358 ± 60 | 186 ± 28 | 194 ± 47 |
| 34 | 1735 ± 230 | 314 ± 48 | 446 ± 93 | 193 ± 34 | 196 ± 58 |
| 37 | 2005 ± 289 | 315 ± 50 | 505 ± 116 | 227 ± 41 | 199 ± 65 |
| 41 | | 329 ± 54 | 530 ± 113 | 228 ± 51 | 201 ± 70 |
| 44 | | 294 ± 48 | 468 ± 99 | 234 ± 52 | 152 ± 48 |
| 48 | | 228 ± 29 | 472 ± 120 | 206 ± 39 | 165 ± 55 |

| | Group 6 | | Group8 | Group 9 |
|---|---|---|---|---|
| | Fulvestrant + | | Fulvestrant + | Fulvestrant + |
| | Formula (I) | Group7 | Formula (I) | Formula (I) |
| | 3 mg/dose + | Fulvestrant | 1 mg/dose + | 1 mg/dose + |
| | 30 mg/kg | 1 mg/dose | 10 mg/kg | 30 mg/kg |
| Days | QW + QD | QW | QW + QD | QW + QD |
| 0 | 173 ± 12 | 173 ± 12 | 173 ± 11 | 173 ± 11 |
| 3 | 182 ± 13 | 206 ± 10 | 196 ± 17 | 181 ± 10 |
| 6 | 139 ± 13 | 272 ± 20 | 187 ± 20 | 149 ± 12 |
| 9 | 106 ± 11 | 315 ± 33 | 193 ± 22 | 139 ± 13 |
| 13 | 100 ± 5 | 389 ± 50 | 207 ± 28 | 131 ± 17 |
| 16 | 101 ± 7 | 472 ± 47 | 215 ± 29 | 129 ± 16 |
| 20 | 91 ± 9 | 595 ± 87 | 246 ± 39 | 115 ± 16 |
| 23 | 90 ± 9 | 623 ± 93 | 242 ± 47 | 103 ± 18 |
| 27 | 95 ± 9 | 697 ± 104 | 258 ± 45 | 101 ± 14 |
| 30 | 81 ± 8 | 727 ± 116 | 267 ± 43 | 97 ± 14 |
| 34 | 85 ± 6 | 829 ± 130 | 294 ± 47 | 83 ± 16 |
| 37 | 74 ± 6 | 856 ± 140 | 283 ± 58 | 91 ± 13 |
| 41 | 72 ± 7 | 859 ± 142 | 305 ± 43 | 87 ± 11 |
| 44 | 69 ± 8 | 808 ± 164 | 275 ± 48 | 75 ± 12 |
| 48 | 55 ± 4 | 623 ± 104 | 226 ± 44 | 59 ± 10 |

1.6.3 Tumor Growth Inhibition Analysis

The inhibition rates of Fulvestrant and the compound of formula (I) on MCF-7 tumors are calculated with tumor size data on PG-D37 respectively and shown in Table 5.

TABLE 5
Tumor growth inhibition analysis

| | | Tumor Size | | | |
|---|---|---|---|---|---|
| | | (mm³) | TGI | T/C | |
| Group | Treatment | on PG-D37 | (%) | (%) | p value |
| 1 | Vehicle, QD | 2005 ± 289 | — | — | — |
| 2 | Fulvestrant, 3 mg/dose, QW | 315 ± 50 | 92.26 | 15.70 | p < 0.0001 |
| 3 | Compound (I), 10 mg/kg, QD | 505 ± 116 | 81.88 | 25.18 | p < 0.0001 |
| 4 | Compound (I), 30 mg/kg, QD | 227 ± 41 | 97.06 | 11.32 | p < 0.0001 |
| 5 | Fulvestrant + Compound (I), 3 | 199 ± 65 | 98.60 | 9.92 | p < 0.0001 |
| | mg/dose + 10 mg/kg, QW + QD | | | | |
| 6 | Fulvestrant + Compound (I), 3 | 74 ± 6 | 105.42 | 3.68 | p < 0.0001 |
| | mg/dose + 30 mg/kg, QW + QD | | | | |
| 7 | Fulvestrant, 1 mg/dose, QW | 856 ± 140 | 62.74 | 42.68 | p < 0.0001 |
| 8 | Fulvestrant + Compound (I), 1 | 283 ± 58 | 94.00 | 14.10 | p < 0.0001 |
| | mg/dose + 10 mg/kg, QW + QD | | | | |
| 9 | Fulvestrant + Compound (I), 1 | 91 ± 13 | 104.46 | 4.54 | p < 0.0001 |
| | mg/dose + 30 mg/kg, QW + QD | | | | |

1.6.4 Tumor Growth Curves

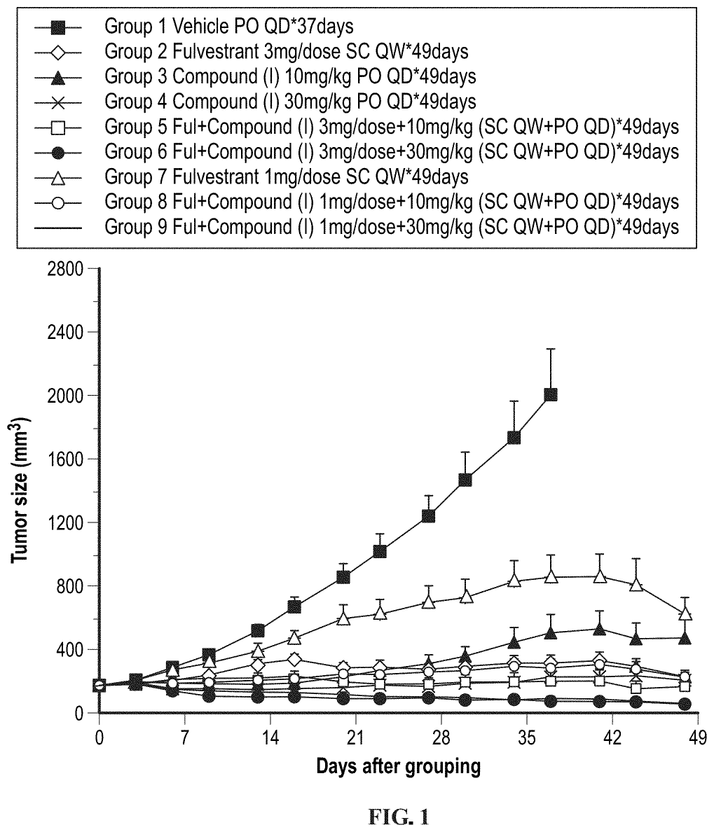

Tumor growth curves are shown in FIG. 1.

1.6.5 Discussion

In this study, the antitumor efficacy and PKPD of fulvestrant and the compound of formula (I) in the treatment of MCF-7 model in female Balb/c nude mice was evaluated. Tumor sizes at various time points are shown in Table 4 and FIG. 1.

The mean tumor size of vehicle treated mice reached 2005 mm³ on PG-D37. When compared with vehicle group, all groups showed significant antitumor activity, the combination of the compound of formula (I) and fulvestrant have improved the efficacy of each single agent. The combination of the compound of formula (I) 30 mg/kg QD dosage with fulvestrant at either 1 mg/dose QW or 3 mg/dose QW caused tumor regression from PG-D6 through the end of the study.

Animal body weight was monitored regularly as an indirect measurement of toxicity. Four mice were euthanized for >20% body weight lost which was caused by E2 pellet burden. One mouse in group 8 lost over 15% body weight. The other animals kept body weight well.

### Example 2: Further Evaluation of the Compound of Formula (I) in a MCF-7 Breast Tumor Model—Monotherapy or Combination with Fulvestrant

The in vivo anti-tumor efficacy of the compound of formula (I) either alone or in combination with fulvestrant on MCF-7 breast tumor model on female Balb/c nude mice was evaluated. The protocol was he same as described in Example 1 with the exception that the dosing schedule of fulvestrant was modified. As shown in Table 6, the compound of formula (I) was dosed orally (po) once daily (QD) and fulvestrant was dosed subcutaneously (sc) once on an ascending dosing schedule for 50 days.

TABLE 6
Study design

| | | | Dose | Dosing Volume | | |
|---|---|---|---|---|---|---|
| Group | n | Treatment | (mg/kg) | (μl/g) | Dosing route | Schedule |
| 1 | 8 | Vehicle | — | 10 μl/g | PO | QD*50 days |
| 2 | 8 | Fulvestrant | 1→3→5 | 20→60→100 | SC | QW*50 days |
| | | | mg/dose | μl/mouse | | |
| 3 | 8 | Compound (I) | 10 mg/kg | 10 μl/g | PO | QD*50 days |
| 4 | 8 | Compound (I) | 30 mg/kg | 10 μl/g | PO | QD*50 days |
| 5 | 8 | Fulvestrant + | 1→3→5 | 20→60→100 | SC + PO | QW + |
| | | Compound (I) | mg/dose + | μl/mouse + | | QD*50 days |
| | | | 10 mg/kg | 10 μl/g | | |
| 6 | 8 | Fulvestrant + | 1→3→5 | 20→60→100 | SC + PO | QW + |
| | | Compound (I) | mg/dose + | μl/mouse + | | QD*50 days |
| | | | 30 mg/kg | 10 μl/g | | |

Fulvestrant: 1 mg/dose (D 1-13), 3 mg/dose (D 14-20), 5 mg/dose (D 21-50).

Animal body weight was monitored regularly as an indirect measurement of toxicity. Supplemented diet was provided for all groups to help body weight kept well. In this study, four mice were euthanized or died because of urinary system abnormal caused by E2 pellet. All other animals kept the body weight well. Body weight changes were minimal, showing the compound of formula (I) did not display toxicity.

The inhibition rates of the compound of formula (I) and fulvestrant on MCF-7 tumors are calculated with tumor size data on PG-D49. The calculation results are shown in Table 7.

TABLE 7
Tumor growth inhibition analysis

| | | Tumor Size | TGI | T/C | |
|---|---|---|---|---|---|
| Group | Treatment | (mm³) | (%) | (%) | p value |
| 1 | Vehicle | 1181 | — | — | — |
| 2 | Fulvestrant | 451 | 71.50 | 38.19 | p < 0.001 |
| | 1/3/5 mg/dose | | | | |
| 3 | Compound (I) | 611 | 55.83 | 51.74 | p < 0.01 |
| | 10 mg/kg | | | | |
| 4 | Compound (I) | 170 | 99.02 | 14.39 | p < 0.001 |
| | 30 mg/kg | | | | |
| 5 | Ful + Compound (I) | 156 | 100.39 | 13.21 | p < 0.001 |
| | 1/3/5 mg/dose + | | | | |
| | 10 mg/kg | | | | |
| 6 | Ful + Compound (I) | 50 | 110.77 | 4.23 | p < 0.001 |
| | 1/3/5 mg/dose + | | | | |
| | 30 mg/kg | | | | |

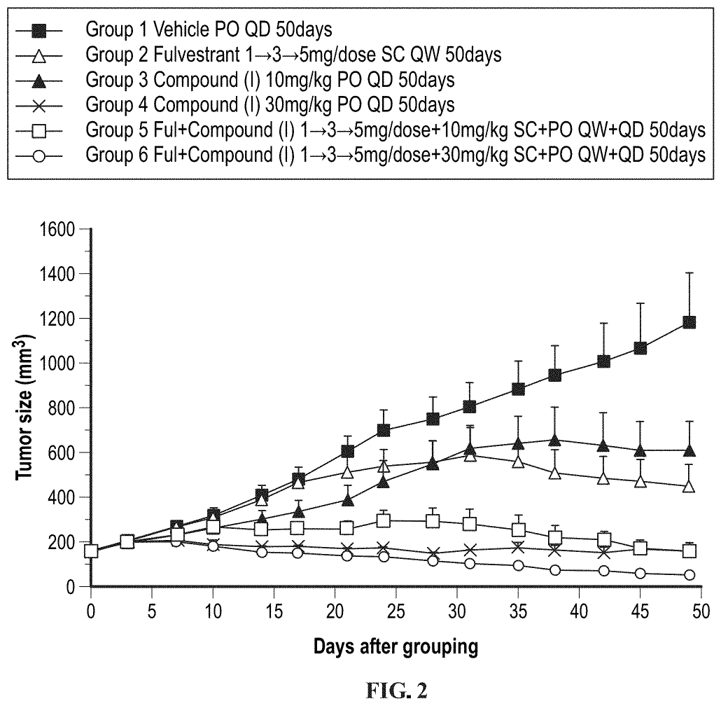

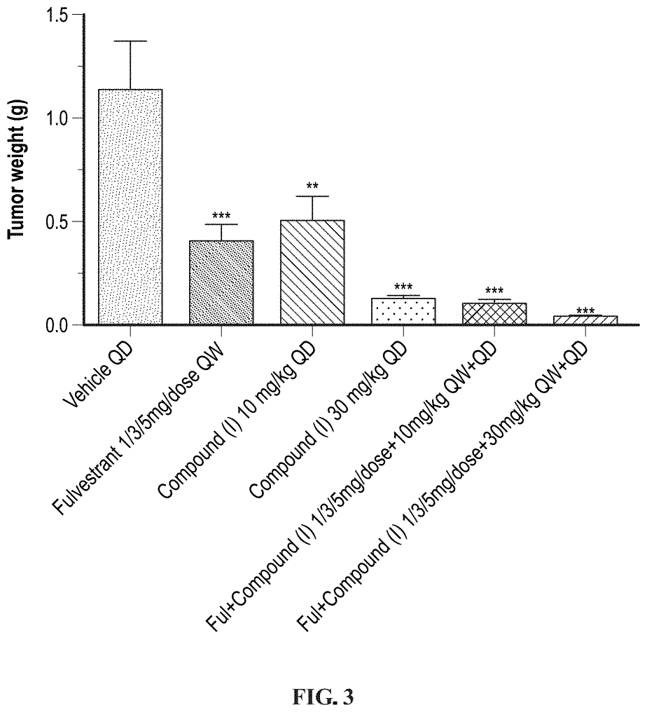

In this study, the antitumor efficacy of ascending doses of fulvestrant, the compound of formula (I), and combinations thereof, in the treatment of MCF-7 model in female Balb/c nude mice was evaluated. Tumor growth curves and tumor weights following administration are shown in FIGS. 2 and 3, respectively. When compared with vehicle group, all the treatment groups showed significant anti-tumor activity. As in Example 1, the combination of the compound of formula (I) and fulvestrant improved the efficacy of the compound of formula (I) as a single agent.

### Example 3: Evaluation of the Compound of Formula (I) in a 22Rv1 Prostate Tumor Model—Monotherapy or Combination with Enzalutamide

3.1 Study

The in vivo anti-tumor efficacy of the compound of formula (I) either alone or in combination with enzalutamide prostate tumor model on male Balb/c nude mice was evaluated. The cell line is derived from a xenograft that was serially propagated in mice after castration—induced regression and relapse of the parental, androgen-dependent CWR22 xenograft. See Sramkoski et al., (1999) In Vitro Cell Dec that Anim, 35(7); 403-409, which is hereby incorporated by reference.

3.2 Experimental Design

In the study, the compound of formula (I) was dosed orally (po) once daily (QD) and enzalutamide, when administered, was dosed orally once daily for 25 days, as shown in Table 8.

TABLE 8
Study design

| | | | | Dosing | | | |
|---|---|---|---|---|---|---|---|
| | | | Dose | Volume | | Dosing | |
| Group | n | Treatment | (mg/kg) | (μl/g) | | Route | Schedule |
| 1 | 8 | Vehicle | — | 10 | μl/g | PO | QD*25 days |
| 2 | 8 | Enzalutamide | 30 | 10 | μl/g | PO | QD*25 days |
| 3 | 8 | Compound of | 10 | 10 | μl/g | PO | QD*25 days |
| | | formula (I) | | | | | |
| 4 | 8 | Compound of | 30 | 10 | μl/g | PO | QD*25 days |
| | | formula (I) | | | | | |
| 5 | 8 | Compound of | 10 + 30 | 10 + 10 | μl/g | PO + PO | QD + |
| | | formula (I) + | | | | | QD*25 |
| | | enzalutamide | | | | | days |
| 6 | 8 | Compound of | 30 + 30 | 10 + 10 | μl/g | PO + PO | QD + |
| | | formula (I) + | | | | | QD *25 |
| | | enzalutamide | | | | | days |

3.3 Materials

3.3.1 Animals

Male Balb/c nude mice with the following characteristics were used in the study:
Species: Mus musculus
Strain: Balb/c nude
Age: 6-8 weeks
Sex: Male
Body weight: 18-22 g
Number of animals: 108 mice
Animal supplier: Beijing Vital River Laboratory Animal Co., LTD.

3.3.2 Housing Conditions

The mice were kept in individual ventilation cages at constant temperature and humidity.
Temperature: 20-26° C.
Humidity: 40-70%.
Cages: Made of polycarbonate. The size is 300 mm×180 mm×150 mm. The bedding material is corn cob, which is changed twice per week.
Diet: Animals had free access to irradiation sterilized dry granule food during the entire study period.
Water: Animals had free access to sterile drinking water.
Cage identification: The identification labels for each cage contained the following information: number of animals, sex, strain, data received, treatment, study number, group number and the starting date of the treatment.
Animal identification: Animals were marked by ear coding.

3.4 Experimental Methods and Procedures

3.4.1 Cell Culture

The 22Rv1 tumor cells were maintained in vitro in RPMI1640 medium supplemented with 10% heat inactivated fetal bovine serum at 37° C. in an atmosphere of 5% CO₂ in air. The tumor cells were routinely subcultured twice weekly. The cells growing in an exponential growth phase were harvested and counted for tumor inoculation.

3.4.2 Tumor Inoculation and Group Assignment

Each mouse was inoculated subcutaneously at the right flank with 22Rv1 tumor cells (5*10⁶) with 50% Matrigel for tumor development. The treatments were started on day 7 after tumor inoculation when the average tumor volume reached 94 mm³. A total of 48 mice were selected and assigned into 6 groups. The testing articles were administrated to the mice according to Table 8.

3.4.3 Testing Articles Formulation Preparation

Formulations for administration were prepared as shown in Table 9.

TABLE 9
Formulation preparation

| Compounds | Preparation | Concentration | Storage |
|---|---|---|---|
| Vehicle | 0.5% HPMC + 0.1% Tween | — | 4° C. |
| Compound | Suspend 22.19 mg | 1 mg/mL | 4° C. |
| of formula | compound (I) in 22.079 mL | | |
| (I) | Vehicle, vortex and stir | | |
| | to obtain a white uniform | | |
| | suspension. | | |
| Compound | Suspend 66.57 mg | 3 mg/mL | 4° C. |
| of formula | compound (I) in 22.079 mL | | |
| (I) | Vehicle, vortex and stir | | |
| | to obtain a white uniform | | |
| | suspension. | | |
| Enzalutamide | Suspend 59.79 mg | 3 mg/mL | 4° C. |
| | Enzalutamide in 19.872 | | |
| | mL 0.5% HPMC, vortex | | |
| | and stir to obtain a | | |
| | white uniform suspension. | | |

3.4.4 Tumor Measurements and Endpoints

The major endpoint was to see if the tumor growth could be delayed or mice could be cured. Tumor size was measured twice weekly in two dimensions using a caliper, and the volume was expressed in mm³ using the formula: V=0.5 a×b² where a and b are the long and short diameters of the tumor, respectively. The tumor sizes are then used for the calculation of T/C and TGI values.

The T/C value (in percent) is an indication of antitumor effectiveness; T and C are the mean volume of the treated and control groups, respectively, on a given day.

TGI was calculated for each group using the formula: TGI (%)=[1−(Ti−T0)/(Vi−V0)]×100; Tᵢ is the average tumor volume of a treatment group on PG-Di, T₀ is the average tumor volume of the treatment group on the day of treatment start, Vᵢ is the average tumor volume of the vehicle control group on the same day with Tᵢ, and V₀ is the average tumor volume of the vehicle group on the day of treatment start.

All groups were taken down on PG-D24 and PG-D25 when the average tumor size of vehicle group reached 2305 mm³.

3.4.5 Statistical Analysis

Summary statistics, including mean and the standard error of the mean (SEM), are provided for the tumor volume of each group at each time point. For comparison among groups, a one-way ANOVA was performed. All the analysis were conducted using GraphPad Prism software. p<0.05 was considered to be statistically significant.

3.4.6 Sample Collection

Plasma and tumor samples were collected.
1) Plasma collection: The blood was collected and placed in K2-EDTA tubes, centrifuged at 2,000×g (4,600 rpm), 4° C. for 15 min. Plasma was isolated and stored at −80° C.
2) Tumor collection: Each tumor was split into 2 pieces, one for PD purpose, one for PK analysis. The samples were snap-frozen in liquid nitrogen and then transferred to −80° C. for storage.

3.5 Results

3.5.1 Body Weight Gain or Loss

Animal body weight was monitored regularly as an indirect measurement of toxicity. Supplemented diet was provided for group 5 and 6 to help body weight maintenance.

One mouse in group 6 was euthanized for more than 20% body weight loss, all other animals tolerated treatments well. Body weight changes in male Balb/c nude mice bearing 22Rv1 model were minimal.

3.5.2 Tumor Volume Measurement

Mean tumor volume over time of male Balb/c nude mice bearing 22Rv1 tumors are shown in Table 10.

TABLE 10
Tumor volume measurement (mm³, mean ± SEM)

| | | | | | Group 5 | Group 6 |
|---|---|---|---|---|---|---|
| | | | Group 3 | Group 4 | Compound | Compound |
| | Group 1 | Group 2 | Compound | Compound | (I) + Enza | (I) + Enza |
| | Vehicle | Enzalutamide | (I) | (I) | 10 + 30 | 30 + 30 |
| | — | 30 mg/kg | 10 mg/kg | 30 mg/kg | mg/kg | mg/kg |
| Days | QD | QD | QD | QD | QD + QD | QD + QD |
| 0 | 95 ± 6 | 94 ± 4 | 94 ± 5 | 94 ± 4 | 94 ± 3 | 94 ± 5 |
| 3 | 187 ± 19 | 177 ± 13 | 154 ± 12 | 131 ± 13 | 141 ± 11 | 125 ± 15 |
| 7 | 364 ± 29 | 345 ± 23 | 257 ± 28 | 165 ± 25 | 263 ± 28 | 155 ± 28 |
| 10 | 609 ± 46 | 507 ± 53 | 378 ± 40 | 188 ± 30 | 346 ± 33 | 189 ± 41 |
| 14 | 1002 ± 121 | 825 ± 65 | 598 ± 58 | 289 ± 40 | 565 ± 62 | 295 ± 51 |
| 17 | 1293 ± 116 | 1115 ± 65 | 825 ± 87 | 398 ± 60 | 768 ± 96 | 404 ± 73 |
| 21 | 1689 ± 143 | 1531 ± 143 | 1152 ± 114 | 546 ± 67 | 1001 ± 107 | 492 ± 77 |
| 24 | 2305 ± 216 | 1814 ± 186 | 1409 ± 102 | 653 ± 64 | 1200 ± 117 | 638 ± 101 |

3.5.3 Tumor Growth Inhibition Analysis

The inhibition rates of enzalutamide and the compound of formula (I) on 22Rv1 tumors are calculated with tumor size data on PG-D24 respectively and shown in Table 11.

TABLE 11
Tumor growth inhibition analysis

| | | Tumor Size | TGI | T/C | |
|---|---|---|---|---|---|
| Group | Treatment | (mm³) | (%) | (%) | p value |
| 1 | Vehicle, QD | 2305 ± 216 | — | — | — |
| 2 | Enzalutamide, | 1814 ± 186 | 22.17 | 78.69 | p > 0.05 |
| | 30 mg/kg, QD | | | | |
| 3 | Compound (I), | 1409 ± 102 | 40.49 | 61.13 | p < 0.001 |
| | 10 mg/kg, QD | | | | |
| 4 | Compound (I), | 653 ± 64 | 74.71 | 28.34 | p < 0.001 |
| | 30 mg/kg, QD | | | | |
| 5 | Compound (I) + | 1200 ± 117 | 49.98 | 52.05 | p < 0.001 |
| | Enza, 10 + 30 | | | | |
| | mg/kg, | | | | |
| | QD + QD | | | | |
| 6 | Compound (I) + | 638 ± 101 | 75.38 | 27.69 | p < 0.001 |
| | Enza, 30 + 30 | | | | |
| | mg/kg, | | | | |
| | QD + QD | | | | |

3.5.4 Tumor Growth Curves

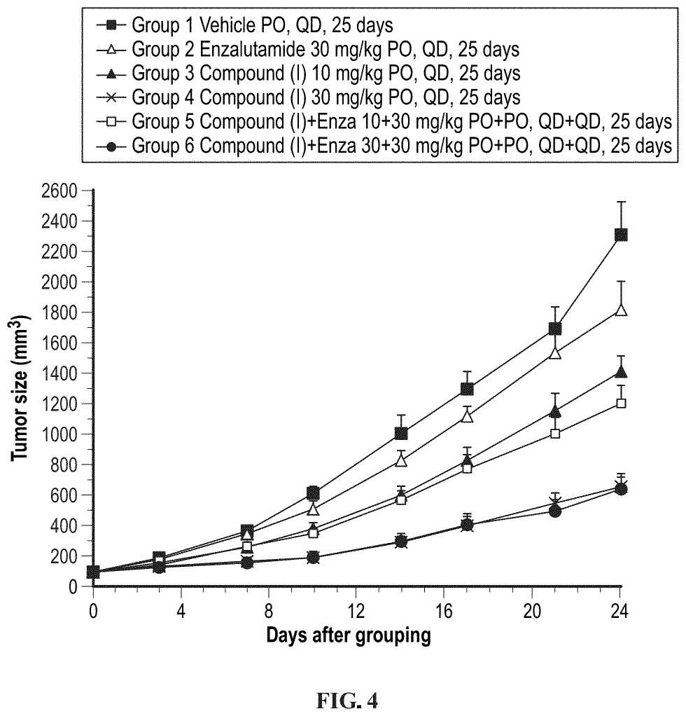

Tumor growth curves are shown in FIG. 4.

3.5.5 Tumor Weight Bar Graphs

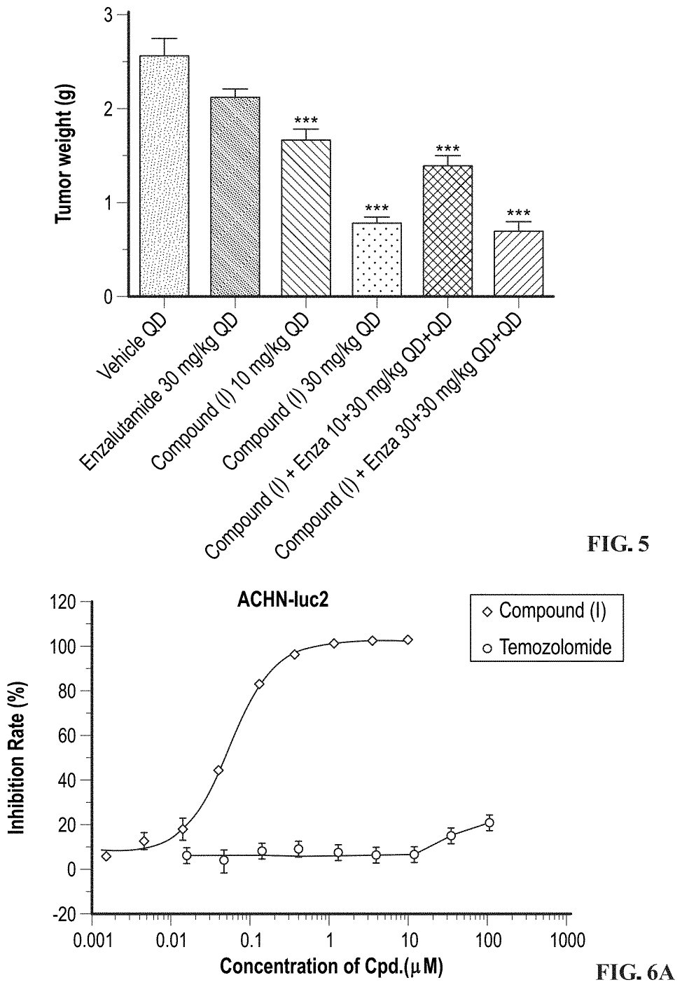

Tumor weights are shown in FIG. 5.

3.5.6 Discussion

In this study, the antitumor efficacy of the compound of formula (I) and enzalutamide in the treatment of 22Rv1 model in male Balb/c nude mice was evaluated. The mean tumor size of vehicle treated mice reached 2305 mm³ on PG-D24. When compared with vehicle group, all groups except enzalutamide single treatment group showed significant antitumor activity. Treatment with the compound of formula (I) 10 mg/kg QD with enzalutamide 30 mg/kg QD demonstrated slightly improved antitumor activity to treatment with formula (I) 10 mg/kg QD alone. Treatment with the compound of formula (I) 30 mg/kg QD with enzalutamide 30 mg/kg QD demonstrated comparable antitumor activity to treatment with formula (I) 30 mg/kg QD alone.

Animal body weight was monitored regularly as an indirect measurement of toxicity. Supplemented diet was provided to the last 2 groups to help body weight maintenance. One mouse in group 6 was euthanized for more than 20% body weight loss. All other mice kept body weights well.

### Example 4: Evaluation of the Compound of Formula (I) in Seven Cell Metastasis Cell Lines

4.1 Study

The anti-proliferation effect of 2 compounds (the compound of formula (I) and temozolomide) in 7 CNS metastasis cell lines (MOLT-4-luc, Nalm-6-luc, NCI-H460-luc, ACHN-luc2, PC-9-luc, H1975-luc, and KM12-luc) was evaluated.

4.2 Experimental Design

The plating of cells and compound treatments are shown below in the plate map (Table 12). One plate was needed for the test of 2 compounds (the compound of formula (I) and temozolomide) in one cell line. The compound of formula (I) was tested from 10 μM, 3-fold serial dilution. Temozolomide was tested from 100 μM, 3-fold serial dilution.

TABLE 12
Plate map

| | 1 | 2 | 3 | 4 | 5 | 6 | 7 | 8 | 9 | 10 | 11 | 12 |
|---|---|---|---|---|---|---|---|---|---|---|---|---|
| A | PBS | PBS | PBS | PBS | PBS | PBS | PBS | PBS | PBS | PBS | PBS | PBS |
| B | Blank, | Compound of formula (I), top concentration: 10 μM, 3-fold | | | | | | | | | Vehicle | PBS |
| C | culture | serial dilution, 9 points, triplicate | | | | | | | | | control, | PBS |
| D | medium | | | | | | | | | | cells | PBS |
| E | only | Temozolomide, top concentration: 100 μM, 3-fold serial | | | | | | | | | without cpd | PBS |
| F | | dilution, 9 points, triplicate | | | | | | | | | | PBS |
| G | | | | | | | | | | | | PBS |
| H | PBS | PBS | PBS | PBS | PBS | PBS | PBS | PBS | PBS | PBS | PBS | PBS |

4.3 Materials

4.3.1 Cell Lines

Table 13 shows a list of cell lines used in the example.

TABLE 13
Cell line information

| | | | | Culture | Culture |
|---|---|---|---|---|---|
| Cell Line | Cancer Type | Vendor | Cat. No. | Properties | Medium |
| MOLT-4-luc | Hematopoietic | Caliper | NA | Suspension | RPMI-1640 + 10% FBS |
| Nalm-6-luc | Hematopoietic | WuXi | NA | Suspension | RPMI-1640 + 10% FBS |
| NCI-H460-luc | Lung | Caliper | NA | Adherent | RPMI-1640 + 10% FBS |
| ACHN-luc2 | Renal | Caliper | NA | Adherent | EMEM + 10% FBS |
| PC-9-luc | Lung | WuXi | NA | Adherent | RPMI-1640 + 10% FBS |
| H1975-luc | Lung | WuXi | NA | Adherent | RPMI-1640 + 10% FBS |
| KM12-luc | Colon | JCRB | JCRB1389 | Adherent | DMEM + 10% FBS |

4.3.2 Culture Medium

Table 14 shows a list of culture media used in the example.

TABLE 14
Culture Media

| Culture medium or reagent | Vendor | Cat# |
|---|---|---|
| RPMI-1640 | GIBCO | 22400-089 |
| DMEM | GIBCO | 11965-092 |
| EMEM | ATCC | 30-2003 |
| Antibiotic-Antimycotic | GIBCO | 15240-062 |
| FBS | ExCell Bio | FND500 |
| DMSO | SIGMA | D2650 |
| Dulbecco's PBS | Coming | 21-031-CVC |
| 0.25% Trypsin | Gibco | 25200-072 |

4.3.3 Assay Plates

Assay plate: Greiner CELLSTAR® 96 well plates with black wells flat bottom (with lid and micro-clear bottom), #655090.

Compound plate: Nunc-442587, Pinchbar Design Polypropylene, V-shape well bottoms, Sterile.

4.3.4 Cell Viability Reagents and Instruments

Promega CellTiter-Glo Luminescent Cell Viability Assay kit (Promega-G7573).

2104 EnVision Multilabel Reader, PerkinElmer.

4.4 Anti-Proliferation Assay

4.4.1 Cell Culture

All the cell lines were maintained in culture conditions at 37° C. in an atmosphere with 5% CO₂ in air. The tumor cells were routinely subcultured. The cells growing in an exponential growth phase were harvested and counted for plating.

4.4.2 Cell Plating

Cells were counted by haemocytometer with Trypan blue staining. Cell concentration was adopted to proper cell density, as shown in Table 15. Next, 135 μL of cell suspension were plated into the assay plates according to the plate map and 135 μL of assay medium into the blank wells. The plates were incubated at 37° C., 5% CO2, 95% air and 100% relative humidity overnight.

TABLE 15
Cell Density

| Cell Line | Cell Density |
|---|---|
| MOLT-4-luc | 1000 |
| Nalm-6-luc | 800 |
| NCl-H460-luc | 250 |
| ACHN-luc | 500 |
| H1975-luc | 800 |
| KM12-luc | 3000 |
| PC-9-luc | 2500 |

4.4.3 Compound Stock Plate Preparation

Preparation of compound stock plates (1000× stock plates) were prepared by serial dilutions of the stock solutions from the highest concentration to the lowest in DMSO according to the plate map in Table 16.

TABLE 16
Plate layout (μM) of 1000X stock.

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | | | | | | | |
|---|---|---|---|---|---|---|---|---|---|---|
| A | 100000 | 33333 | 11111 | 3704 | 1234 | 412 | 13 | 46 | 15 | Temozolomide |
| B | 10000 | 3333 | 1111 | 370 | 123 | 41 | 14 | 5 | 1.5 | Compound (I) |

4.4.4 Compound Plate (10×) Preparation and Compound Treatment
10× concentrate compound plate preparation: Add 198 μL of assay medium into each well of the V-bottom plate; then transfer 2 μL of the stock compound solution of each concentration from the stock plate. Add 2 μL of DMSO into the Blank and Control wells. Pipette up and down to mix well. This V-plate is designated as the 10× concentrate compound plate.
For day 0 plate, add 15 μL of the DMSO-medium into the Control wells. The final DMSO concentration was 0.1%. Then directly proceed to cell viability assay.
For assay plate, add 15 μL of the compound-medium of each well from the 10× concentrate compound plate into the cells in 96-well assay plate according to the plate map. Add 15 μL of the DMSO-medium into the Blank and Control wells. The final DMSO concentration was 0.1%.
Return the assay plate into incubator and incubate for 6 days.

4.4.5 CellTiter-Glo Luminescent Cell Viability Assay

The procedures were performed according to the Promega CellTiter-Glo Luminescent Cell Viability Assay Kit manual (Promega-G7573).
The CellTiter-Glo buffer was thawed and equilibrated to room temperature prior to use.
CellTiter-Glo Substrate was equilibrated to room temperature prior to use.
The entire liquid volume of CellTiter-Glo Buffer was transferred into the amber bottle containing CellTiter-Glo Substrate to reconstitute the lyophilized enzyme/substrate mixture. This forms the CellTiter-Glo Reagent.
The contents were mixed by gently vortexing to obtain a homogeneous solution.
The plate was equilibrated and its contents to room temperature for approximately 30 minutes. 75 μL (equal to the half volume of culture medium present in each well) CellTiter-Glo
Reagent was added in each well. Plates were covered with aluminum foil to protect from light.
The contents were mixed for 2 minutes on an orbital shaker to induce cell lysis.
The plate was incubated at room temperature for 20 minutes to stabilize luminescent signal.
Record luminescence on the 2104 EnVision plate reader.

4.4.6 Data Analysis

Inhibition rate (IR) of the tested compounds was determined by the following formula: IR (%)=(1−(RLU compound−RLU day0)/(RLU control−RLU day0))*100%. The inhibitions of different dose of compound were calculated in Excel file, and then were used to plot inhibition curves and evaluate related parameters, such as Min (%), Max (%) and GI50. The data were interpreted by GraphPad Prism.

4.5 Results

Table 17 shows a summary of the anti-proliferation results for the compound of formula (I) and temozolomide in the cell viability assay.

TABLE 15
The compound GI50 values in cell viability assay.

| | | | | | Fold |
|---|---|---|---|---|---|
| | | | | Absolute | Growth |
| | | Min | Max | GI50 | (Day 6/ |
| Cell Line | Compound ID | (%) | (%) | (μM) | Day 0) |
| ACHN-luc2 | Compound (I) | 6.0 | 102.9 | 0.047 | 34.02 |
| | Temozolomide | 2.8 | 20.0 | >100 | |
| NCI-H460-luc | Compound (I) | 1.9 | 101.1 | 0.228 | 80.17 |
| | Temozolomide | 4.8 | 7.2 | >100 | |
| H1975-luc | Compound (I) | −2.3 | 105.8 | 0.226 | 17.92 |
| | Temozolomide | −0.3 | 13.0 | >100 | |
| KM12-luc | Compound (I) | −15.2 | 111.2 | 0.654 | 9.80 |
| | Temozolomide | −12.1 | 1.6 | >100 | |
| MOLT-4-luc | Compound (I) | −18.4 | 103.5 | 0.200 | 28.79 |
| | Temozolomide | −2.2 | 23.2 | >100 | |
| Nalm-6-luc | Compound (I) | −20.8 | 105.6 | 0.154 | 18.49 |
| | Temozolomide | 6.5 | 22.9 | >100 | |
| PC-9-luc | Compound (I) | −13.7 | 116.3 | 2.787 | 7.06 |
| | Temozolomide | −13.1 | 5.6 | >100 | |

4.6 Discussion

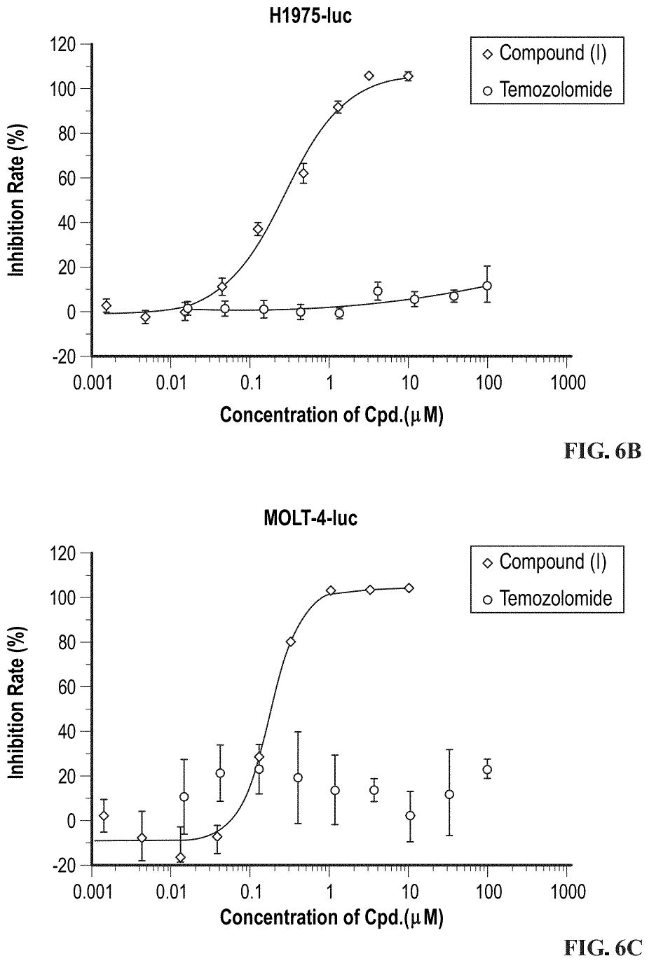

Inhibition curves in the cell viability assays for the various cell lines are shown in FIGS. 6A-G. The results showed that the compound of formula (I) could show significant inhibitory effect in 7 cell lines (MOLT-4-luc, Nalm-6-luc, NCI-H460-luc, ACHN-luc2, PC-9-luc, H1975-luc, and KM12-luc) with absolute GI50 values ranging from 0.047 μM to 2.787 μM. However, temozolomide didn't show obvious inhibitory effect at current testing concentrations.

### Example 5: Evaluation of the Compound of Formula (I) in Seven Cell Melanoma Cell Lines

The anti-proliferation effect of the compound of formula (I) in 7 CNS melanoma cell lines was evaluated. Assays were performed as in Example 4 but with the cell lines shown in Table 18.

TABLE 18
Cell line information

| | | | | Culture | Culture |
|---|---|---|---|---|---|
| Cell Line | Cancer Type | Vendor | Cat. No. | Properties | Medium |
| A375 | Melanoma | ATCC | CRL-1619 | adherent | DMEM + 10% FBS |
| COLO 829 | Melanoma | ATCC | CRL-1974 | adherent | DMEM/F12 + 10% FBS |
| SK-MEL-24 | Melanoma | ATCC | HTB-71 | adherent | EMEM + 15% FBS |
| SK-MEL-28 | Melanoma | ATCC | HTB-72 | adherent | EMEM + 10% FBS |
| SK-MEL-3 | Melanoma | ATCC | HTB-69 | adherent | McCoy's 5a + |
| | | | | | 15% FBS |
| SK-MEL-31 | Melanoma | ATCC | HTB-73 | adherent | EMEM + 0.1 mM |
| | | | | | NEAA + 1.0 mM |
| | | | | | sodium pyruvate + |
| | | | | | 15% FBS |
| SK-MEL-5 | Melanoma | ATCC | HTB-70 | adherent | EMEM + 10% FBS |

Table 20 shows the anti-proliferative results of the compound of formula (I) in cell viability assays for the various cell lines tested. The results show that the compound of formula (I) shows significant inhibitory effects in 7 melanoma cell lines.

TABLE 20
The compound GI50 values in cell viability assay.

| | | Min | Max | Absolute |
|---|---|---|---|---|
| Cell Line | Compound ID | (%) | (%) | GI50 (μM) |
| A375 | Compound (I) | −0.12 | 101.21 | 0.156 |
| COLO 829 | Compound (I) | −3.11 | 107.94 | 0.875 |
| SK-MEL-3 | Compound (I) | −1.51 | 111.93 | 0.174 |
| SK-MEL-5 | Compound (I) | −2.91 | 116.12 | 1.239 |
| SK-MEL-24 | Compound (I) | 1.36 | 113.43 | 0.151 |
| SK-MEL-28 | Compound (I) | 1.70 | 106.21 | 0.152 |
| SK-MEL-31 | Compound (I) | −0.53 | 125.31 | 1.228 |

In this study, the anti-proliferative effect of the compound of Formula (I) and temozolomide on 7 CNS metastasis cell lines was evaluated. The compound of formula (I) showed significant inhibitory effects on 7 cell lines (A375, COLO 829, SK-MEL-3, SK-MEL-5, SK-MEL-24, SK-MEL-28, and SK-MEL-31) with absolute GI50 values ranging from 0.156 uM to 1.239 uM.

### Example 6: Evaluation of the Brain Distribution of the Compound of Formula (I) Following Oral Administration

6.1 Study

The pharmacokinetics and brain distribution in maleCD1 mice and Sprague-Dawley rats following a single oral (PO) dose was evaluated. The compound of formula (I) was administered once at 10 mg/kg to mice and at 1, 10, 30, and 100 mg/kg to rats. All doses, compound of formula (I) concentrations, and derived PK parameters are expressed as the free base of the compound of formula (I).

6.2 Materials and Methods

6.2.1 Test System

The animals used in this study are described in Table 21. Animals were group-housed by species and individually identified by tail mark.

TABLE 21
Test System

| | | |
|---|---|---|
| Species: | Mus musculus | Rattus norvegicus |
| Strain: | CD1 | Sprague-Dawley |
| Sex: | Male | Male |
| Source: | Charles River Laboratories | Charles River Laboratories |

6.2.2 Experimental Design

The in-life portion of these studies was performed by Alliance Pharma (Malvern, Pa.) according to Protocols 200107-02 (mice) and 1910158-02 (rats). Animals were assigned randomly to the dose groups and were fasted 12 hours prior to dosing. Water was available ad libitum. The compound of formula (I) (as an HCl salt) was given to male CD1 mice at 10 mg/kg single PO dose, while in Sprague-Dawley rats, the doses were 1, 10, 30, and 100 mg/kg. In both studies, the compound of formula (I) was formulated in 0.5% hydroxypropyl methylcellulose (HPMC)/0.2% Tween 80 and the dosing volume was 5 mL/kg for all dose groups.

6.2.3 Sample Collection and Handling

Whole blood was collected at 2, 4, 6, and 12 hours post dose from mice and predose and 1, 2, 4, 8, 12, and 24 hours post dose from rats into tubes containing dipotassium ethylenediaminetetraacetic acid (K2EDTA). Blood was obtained from 3 mice and 4 rats per time-point per dose level. The blood samples were processed into plasma analyzed at Alliance Pharma using the method as described below.

The brain tissue was collected from mice at 2, 4, 6, and 12 hours post dose and was collected from rats at 6 hours post dose given 1, 10, 30, or 100 mg/kg of the compound of formula (I). Brain tissue was obtained from 3 mice per time-point at the 10 mg/kg dose level and 4 rats per dose level at the 6-hour post dose time-point.

Brain tissue was rinsed by phosphate-buffered saline (PBS) buffer immediately after harvest, weighed, and mixed with solvent (MeOH:H₂O [volume over volume of 20:80]) with a ratio of tissue to solvent of 1:4 (weight over volume). Brain tissue was then homogenized using a GenoGrinder (Module #2010) with the addition of ZIRCONIA/SILICA beads (1.0 mm diameter, Catalog No. 11079110z). After homogenization, the homogenate was aliquoted out for sample analysis.

6.2.4 Quantitation of the Compound of Formula (I)

The concentrations of the compound of formula (I) in the plasma and brain homogenate were determined versus a calibration curve prepared in plasma and naïve brain homogenate, respectively. Samples were deproteinized with acetonitrile containing tolbutamide as internal standard. After centrifugation, the supernatants were analyzed by liquid chromatography with tandem mass spectrometry (LC-MS/MS). Chromatography was performed with a Zorbax SB-C18 (50×2.1 mm, 5 μM) column under gradient conditions described in Table 22. Mobile Phase A (0.1% formic acid [FA] in water) and Mobile Phase B (0.1% FA in acetonitrile) were delivered as a flow rate of 0.8 mL/min. LC-MS/MS was performed using a positive TurboIonSpray® interface on a SciexAPI 4000 and multiple-reaction monitoring (MRM) over a concentration range of 1 ng/mL to 1000 (rats) or 2000 (mice) ng/mL for the plasma and brain homogenate samples.

TABLE 22
High-Performance Liquid Chromatography
Gradient Elution Scheme

| | |
|---|---|
| Mice | Rats |

| Time | Mobile | Time | Mobile |
|---|---|---|---|
| (min) | Phase B (%) | (min) | Phase B (%) |
| 0.0 | 15 | 0.2 | 20 |
| 0.30 | 15 | 1.3 | 95 |
| 1.10 | 95 | 2 | 95 |
| 2.10 | 95 | 2.05 | 20 |
| 2.15 | 15 | | |
| 3.00 | 15 | | |

6.2.5 Data Analysis

The mean plasma concentration-time data of the compound of formula (I) was used to determine the pharmacokineticparameters by standard noncompartmental methods using Phoenix® WinNonlin (version 8.2.0; Certara, Princeton, N.J.). Graphs were plotted in Prism® (version 2.3.0; GraphPad, San Diego, Calif.). Concentrations were rounded to 3 significant figures.

6.3 Results

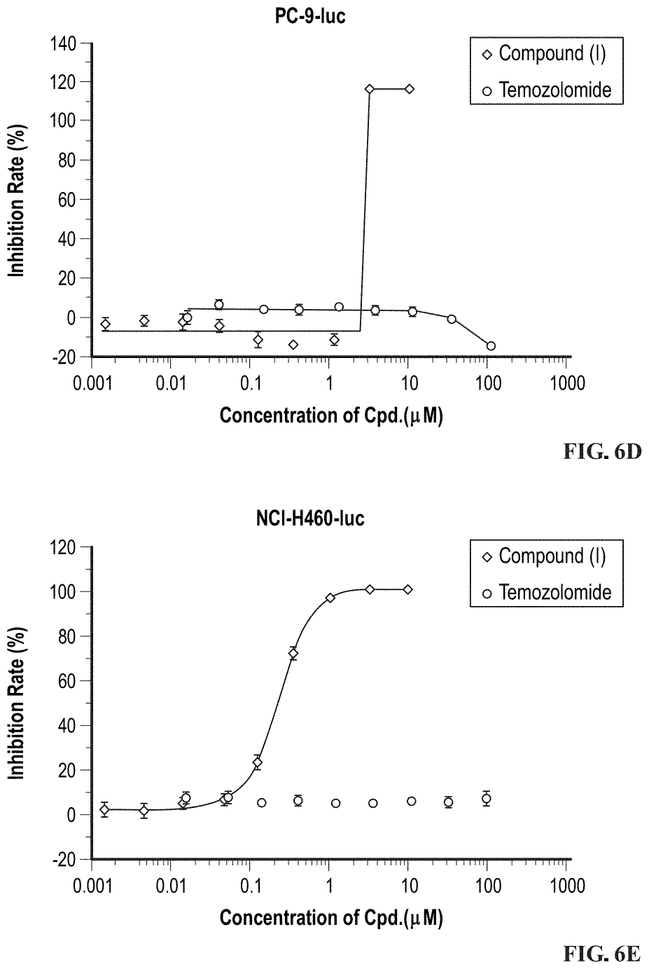

The results obtained from pharmacokinetic analysis of the compound of formula (I) in rats using mean plasma concentrations for each dose group are summarized in Table 23. The mean plasma and brain concentrations and brain-to-plasma concentration ratios in mice and rats are provided in Table 24. Mean plasma and brain concentrations in mice are graphically presented in FIG. 7.

Following 10 mg/kg PO dosing in mice, the brain-to-plasma concentration ratio at 2 and 6 hours post dose was 1.7 and 1.9, respectively. The brain-to-plasma concentration ratio at 6 hours following administration was 6.3, 6.1, 11, and 12 following 1, 10, 30, and 100 mg/kg PO doses in rats.

TABLE 23
Mean Plasma Concentrations of the Compound of Formula (I)

| | | | | |
|---|---|---|---|---|
| Time (h) | 1 mg/kg | 10 mg/kg | 30 mg/kg | 100 mg/kg |

Mean Plasma Concentrations (ng/mL) ^{a}

| 1 | 1.52 | 8.45 | 43.2 | 81.2 |
|---|---|---|---|---|
| 2 | 2.41 | 25.9 | 73.1 | 142 |
| 4 | 4.91 | 51.6 | 136 | 151 |
| 6 | 9.37 | 90.4 | 180 | 243 |
| 8 | 6.42 | 52.5 | 155 | 170 |
| 12 | 4.64 | 46.3 | 117 | 193 |
| 24 | BQL | 12.9 | 66.4 | 224 |
| Parameter | Pharmacokinetics | | | |
| Cₘₐₓ (ng/mL) | 9.37 | 90.4 | 180 | 243 |
| Tₘₐₓ (h) | 6.0 | 6.0 | 6.0 | 6.0 |
| AUC_{inf} | NC | 1077 | 3870 | NC |
| (ng/mL*h) | | | | |
| t_{1/2} (h) | NC | 7.54 | 13.4 | NC |

AUC_{inf}: area under the concentration-time curve from time 0 to infinity; BQL: below the quantitation limit; Cₘₐₓ: maximum observed concentration; NC: not calculated; t_{1/2}: half-life; Tₘₐₓ: time to Cₘₐₓ.
^{a} n = 4.

TABLE 24
Mean Plasma and Brain Concentrations and Brain-to-Plasma
Concentration Ratios of the Compound of Formula (I)

| | | Time-point | Brain Conc | Plasma Conc | Brain:Plasma |
|---|---|---|---|---|---|
| Species | Dose | (h) | (ng/mL) | (ng/mL) | Ratio |
| Mice | 10 | 2 | 309 | 187 | 1.7 (0.2) |
| (n = 3) | 10 | 4 | 436 | 209 | 2.1 (0.5) |
| | 10 | 6 | 464 | 247 | 1.9 (0.6) |
| | 10 | 12 | 345 | 105 | 3.3 (0.3) |
| Rats | 1 | 6 | 58.9 | 9.37 | 6.3 |
| (n = 4) | 10 | 6 | 548 | 90.4 | 6.1 |
| | 30 | 6 | 1970 | 180 | 11 |
| | 100 | 6 | 2800 | 243 | 12 |

Values are presented as mean (standard deviation [SD]) where available.

In this study, the PK and brain distribution of the compound of formula (I) was evaluated in male CD1 mice after a single oral dose of 10 mg/kg, and the brain distribution of the compound of formula (I) was evaluated in male Sprague-Dawley rats after a single oral dose of 1 mg/kg, 10 mg/kg, 30 mg/kg, or 100 mg/kg. After dosing in mice, the brain-to-plasma concentration ratio was greater than 1.5 at 2, 4, 6, and 12 hours post-dose, with a ratio of 3.3 at 12 hours post-dose. After dosing in rats, the brain-to-plasma concentration ratio was greater than 6 at 6 hours post-dose for each dose level. This data demonstrates the ability of the compound of formula (I) to penetrate the blood-brain barrier, and suggests the compound of formula (I) may be particularly effective in treating brain tumors and brain metastases.

### Example 7: Evaluation of the Compound of Formula (I) in the Treatment of U-87MG-Luc Glioblastoma Orthotopic Intracranial Model in Female BALB/c Nude Mice

7.1 Study

The in vivo anti-tumor efficacy of the compound of formula (I) and temozolomide in the treatment of U-87MG-luc glioblastoma orthotopic intracranial tumor model in female BALB/c nude mice was evaluated.

7.2 Experimental Design

The dosing schedule for compound of formula (I) or temozolomide was as shown in Table 25.

TABLE 25
Study Design

| | | | | Dosing | | |
|---|---|---|---|---|---|---|
| | | | Dose | Volume | Dosing | |
| Group | n | Treatment | (mg/kg) | (μl/g) | Route | Schedule |
| 1 | 10 | Vehicle | — | 10 | PO | QD*54 days |
| 2 | 10 | Temozolomide | 1 | 10 | PO | QD × 5/7 |
| | | | | | | days*64 days |
| 3 | 10 | Compound (I) | 1 | 10 | PO | QD*112 days |
| 4 | 10 | Compound (I) | 3 | 10 | PO | QD*112 days |
| 5 | 10 | Compound (I) | 10 | 10 | PO | QD*112 days |
| 6 | 10 | Compound (I) | 30 | 10 | PO | QD*112 days |
| 7 | 10 | Compound (I) | 30 | 10 | IV | QW*6 weeks |

7.3 Materials

7.3.1 Animals

Female Balb/c nude mice with the following characteristics were used in the study:
Species: Mus musculus
Strain: Balb/c nude
Age: 6-56 days
Sex: Female
Body weight: 18-22 g
Number of animals: 70 mice
Animal supplier: Beijing Vital River Laboratory Animal Co., LTD.

7.3.2 Housing Conditions

The mice were kept in individual ventilation cages at constant temperature and humidity.
Temperature: 20-26° C.
Humidity: 40-70%.
Cages: Made of polycarbonate. The size is 300 mm×180 mm×150 mm. The bedding material is corn cob, which is changed twice per week.
Diet: Animals had free access to irradiation sterilized dry granule food during the entire study period.
Water: Animals had free access to sterile drinking water.
Cage identification: The identification labels for each cage contained the following information: number of animals, sex, strain, data received, treatment, study number, group number and the starting date of the treatment.
Animal identification: Animals were marked by ear coding.

7.4 Experimental Methods and Procedures

7.4.1 Cell Culture

U-87MG-luc cells were derived from U-87MG parental cells acquired from ATCC by stably introducing the gene that encodes the luciferase (luc) protein. The U-87MG-luc cells were cultured at 37° C. in an atmosphere of 5% CO2 in air. The tumor cells were routinely subcultured twice weekly. The cells growing in an exponential growth phase were harvested and counted for tumor inoculation.

7.4.2 Tumor Inoculation and Group Assignment

3×10⁵ U-87MG-luc tumor cells were micro-injected into the right lobe (caudate nucleus) of the brain of each mouse. A total of 94 mice were inoculated. Ten days after cell inoculation, bioluminescence were measured by Xenogen IVIS Lumina II machine, 70 mice were selected and randomly assigned into 7 groups based on bioluminescence intensity and bodyweight. The mice were dosed according to Table 25.

7.4.3 Testing Articles Formulation Preparation

Formulations for administration were prepared as shown in Table 26.

TABLE 26
Formulation Preparation

| | | Concentration | |
|---|---|---|---|
| Compounds | Preparation | (mg/ml) | Storage |
| Vehicle | 0.5% HPMC + 0.2% Tween 80 | — | 4° C. |
| Compound (I) | Add 3.65 mg of the compound of formula (I) in 33.796 ml | 0.1 | 4° C. |
| | vehicle, vortex and sonicate to make a clear solution. | | |
| Compound (I) | Add 9.54 mg of the compound of formula (I) in 29.444 ml | 0.3 | 4° C. |
| | vehicle, vortex and sonicate to make a clear solution. | | |
| Compound (I) | Add 32.82 mg of the compound of formula (I) in 30.389 ml | 1 | 4° C. |
| | vehicle, vortex and sonicate to make a clear solution. | | |
| Compound (I) | Add 106.92 mg of the compound of formula (I) in 33 ml | 3 | 4° C. |
| | vehicle, vortex and sonicate to make a clear solution. | | |
| Compound (I) | Add 10.84 mg of the compound of formula (I) in 3.346 ml 30% | 3 | RT |
| | HP-β-CD (saline), adjust to pH 6-7, vortex and sonicate to | | |
| | make aclear solution. | | |
| Temozolomide | Add 3.2 mg Temozolomide in 32 ml vehicle, vortex and | 0.1 | 4° C. |
| | sonicate to make a clear solution. | | |

7.5 Results

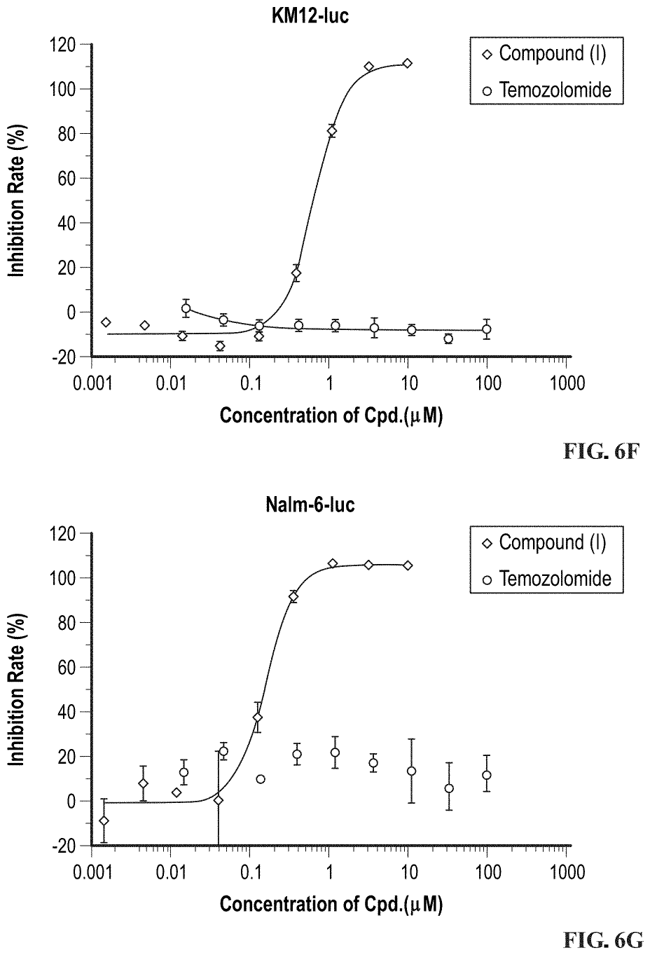

Survival curves are shown in Table 27 and the results are depicted graphically in FIG. 8. Compared to the vehicle treated group, the compound of formula (I) delivered orally (PO) at 10 mg/kg and 30 mg/kg significantly prolonged survival.

TABLE 27
Survival curve analysis

| | | Median survival | |
|---|---|---|---|
| Group | Treatment | (days) | p value |
| 1 | Vehicle PO*QD | 37 | — |
| 2 | Temozolomide 1 mg/kg | 44 | p > 0.05 |
| | PO*QD × 5/7 days | | |
| 3 | Formula (I) | 37 | p > 0.05 |
| | 1 mg/kg PO*QD | | |
| 4 | Formula (I) | 40.5 | p > 0.05 |
| | 3 mg/kg PO*QD | | |
| 5 | Formula (I) | 45 | p < 0.05 |
| | 10 mg/kg PO*QD | | |
| 6 | Formula (I) | 56 | p < 0.0001 |
| | 30 mg/kg PO*QD | | |
| 7 | Formula (I) | 39.5 | p > 0.05 |
| | 30 mg/kg IV*QW | | |

7.6 Discussion

In this study, the anti-tumor efficacy of the compound of formula (I) and temozolomide in U-87 MG-luc glioblastoma orthotopic intracranial tumor model in female BALB/c nude mice was evaluated. Treatment with temozolomide 1 mg/kg PO five times per week, or with the compound of formula (I) at 10 mg/kg PO once daily, improved median survival time by 19% (7 days) and 21% (8 days), respectively, relative to vehicle. Treatment with the compound of formula (I) at a dose of 30 mg/kg PO once daily improved median survival time by 51% (19 days) relative to vehicle.

### Example 8: Evaluation of the Compound of Formula (I) in the Treatment of U-118MG Glioma Subcutaneous Xenograft Model in Female CB17 SCID Mice

8.1 Study

The in vivo anti-tumor efficacy of the compound of formula (I) and temozolomide in the treatment of U-118MG glioma subcutaneous xenograft model in female CB17 SCID (severe combined immunodeficiency) mice was evaluated.

8.2 Experimental Design

The dosing compound of formula (I) or temozolomide was as shown in Table 28.

TABLE 28
Study Design

| | | | | Dosing | | |
|---|---|---|---|---|---|---|
| | | | Dose | Volume | Dosing | |
| Group | n | Treatment | (mg/kg) | (μl/g) | Route | Schedule |
| 1 | 10 | Vehicle | — | 10 | PO | QD*29 days |
| 2 | 10 | Formula (I) | 10 | 10 | PO | QD*29 days |
| 3 | 10 | Formula (I) | 30 | 10 | PO | QD*29 days |
| 4 | 10 | Formula (I) | 60 | 10 | PO | QD*29 days |
| 5 | 10 | Formula (I) | 60 | 10 | PO | QOD*29 days |
| 6 | 10 | Temozolomide | 1 | 10 | PO | 5X/WK*29 days |

8.3 Materials

8.3.1 Animals

Female Balb/c nude mice with the following characteristics were used in the study:
Species: Mus musculus
Strain: CB17 SCID
Age: 6-8 weeks
Sex: Female
Body weight: 18-20 g
Number of animals: 90 mice (plus 30 spare)
Animal supplier: Shanghai Lingchang Laboratory Animal Co., LTD.

8.3.2 Housing Conditions

The mice were kept in individual ventilation cages at constant temperature and humidity.
Temperature: 20-26° C.
Humidity: 40-70%.
Cages: Made of polycarbonate. The size is 300 mm×180 mm×150 mm. The bedding material is corn cob, which is changed twice per week.
Diet: Animals had free access to irradiation sterilized dry granule food during the entire study period.
Water: Animals had free access to sterile drinking water.
Cage identification: The identification labels for each cage contained the following information: number of animals, sex, strain, data received, treatment, study number, group number and the starting date of the treatment.
Animal identification: Animals were marked by ear coding.

8.4 Experimental Methods and Procedures

8.4.1 Cell Culture

U-118MG tumor cells were maintained in vitro in DMEM medium supplemented with 10% fetal bovine serum and 1% anti-anti at 37° C. in an atmosphere of 5% CO2 in air. The tumor cells were routinely subcultured twice weekly. The cells growing in an exponential growth phase were harvested and counted for tumor inoculation.

8.4.2 Tumor Inoculation and Group Assignment

Each mouse was inoculated subcutaneously at the right flank with U-118MG tumor cells (5×10⁶) in 0.2 ml of PBS mixed with Matrigel (50:50) for tumor development. The treatments was started on day 7 after tumor inoculation when the average tumor volume reached 150 mm³. 60 mice were selected and assigned into 6 groups. The testing articles were administrated to the mice according to Table 1.

8.4.3 Testing Articles Formulation Preparation

Formulations for administration were prepared as shown in Table 29.

TABLE 29
Formulation Preparation

| Compounds | Preparation | Concentration | | Storage |
|---|---|---|---|---|
| Vehicle | 0.5% HPMC + 0.2% TWEEN80 | — | | 4° C. |
| Formula (I) | Dissolve 11.87 mg of the compound of formula (I) in 11 | 1 | mg/mL | 4° C. |
| | ml Vehicle, vortex and sonicate to make a clear solution. | | | |
| Formula (I) | Dissolve 35,62 mg of the compound of formula (I) in 11 | 1 | mg/mL | 4° C. |
| | ml Vehicle, vortex and sonicate to make a clear solution. | | | |
| Formula (I) | Dissolve 35.62 mg of the compound of formula (I) in 11 | 6 | mg/mL | 4° C. |
| | ml Vehicle, vortex and sonicate to make a clear solution. | | | |
| Temozolomide | Dissolve 1.1 mg temozolomide in 11 ml Vehicle, stir and | 0.1 | mg/mL | 4° C. |
| | sonicate to make a clear solution. | | | |

8.4.4 Observations

All the procedures related to animal handling, care and the treatment in the study were performed according to the guidelines approved by the Institutional Animal Care and Use Committee (IACUC) of WuXi AppTec following the guidance of the Association for Assessment and Accreditation of Laboratory Animal Care (AAALAC). At the time of routine monitoring, the animals were daily checked for any effects of tumor growth and treatments on normal behavior such as mobility, food and water consumption, body weight gain/loss, eye and any other abnormal effect as stated in the protocol. Death and observed clinical signs were recorded on the basis of the numbers of animals within each subset. Animals that were observed to be in a continuing deteriorating condition or their tumor size exceeding 3000 mm³ were euthanized prior to death or before reaching a comatose state.

8.4.5 Tumor Measurements and Endpoints

Tumor size was measured twice weekly in two dimensions using a caliper, and the volume was expressed in mm³ using the formula: V=0.5 a×b² where a and b are the long and short diameters of the tumor, respectively. The tumor sizes are then used for the calculation of T/C and TGI values.

The T/C value (in percent) is an indication of antitumor effectiveness; T and C are the mean volume of the treated and control groups, respectively, on a given day.

TGI was calculated for each group using the formula: TGI (%)=[1−(Ti−T0)/(Vi−V0)]×100; Ti is the average tumor volume of a treatment group on PG-D28 T0 is the average tumor volume of the treatment group on the day of treatment start, Vi is the average tumor volume of the vehicle control group on the same day with Ti, and V0 is the average tumor volume of the vehicle group on the day of treatment start.

All Groups were taken down on PG-D28, tumor, plasma and brain samples were collected.

8.4.6 Statistical Analysis

Summary statistics, including mean and the standard error of the mean (SEM), are provided for the tumor volume and relative tumor growth of each group at each time point.

For statistical analysis for difference among groups 2-6 and group 1, a one-way ANOVA was performed. All the analysis were conducted using GraphPad Prism software. p<0.05 was considered to be statistically significant.

8.4.7 Sample Collection and Analysis

Plasma, tumor and brain samples were collected at pre-dose, 1 h, 2 h, 4 h, 6 h and 24 h.
1) Plasma collection: The blood was collected and placed in K2-EDTA tubes, centrifuged at 2,000×g (4,600 rpm), 4° C. for 15 min. Plasma was isolated and stored at −80° C.
2) Tumor collection: Each tumor was split into 2 pieces, one for PK, one for PD. Samples were snap-frozen in liquid nitrogen and then transferred to −80° C. for storage.
3) Brain collection: Whole brain was snap-frozen in liquid nitrogen and then transferred to −80° C. for storage.

8.5 Results

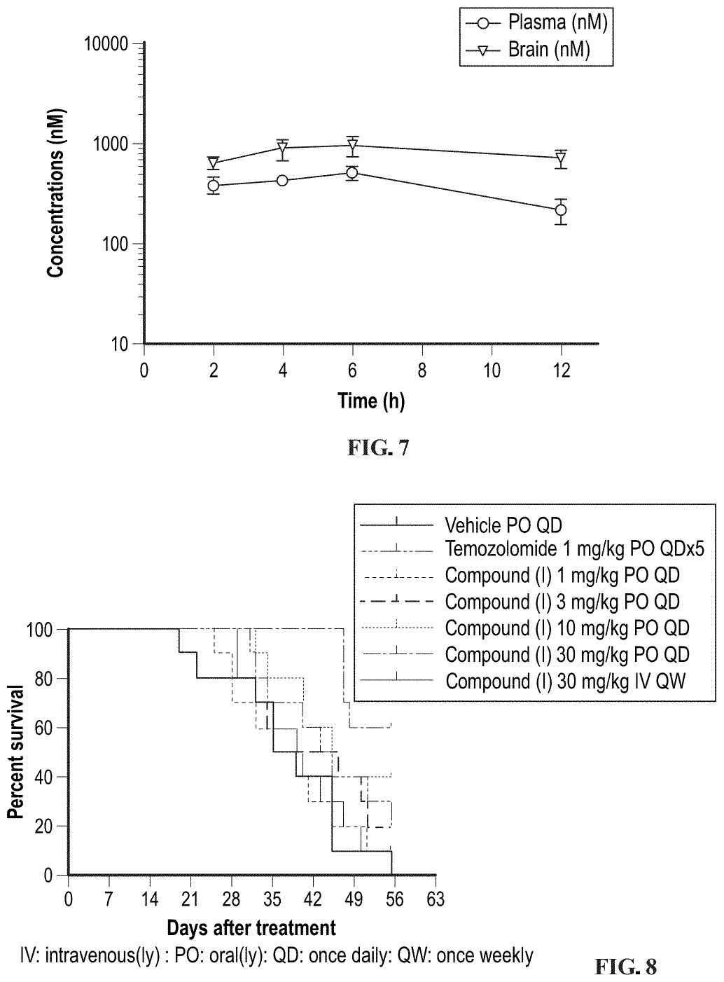

Mean Tumor volume overtime of female CB17 SCID nude mice bearing U-118MG xenograft tumors are shown in Table 30. Tumor growth curves are shown in FIG. 9.

TABLE 30
Tumor Volume Measurement (mm³, mean ± SEM)

| | Group | | | | | |
|---|---|---|---|---|---|---|
| | 1 | 2 | 3 | 4 | 5 | 6 |
| | Vehicle | Comp (I) | Comp (I) | Comp (I) | Cimp (I) | Temozolomide |
| | — | 10 mg/kg | 30 mg/kg | 60 mg/kg | 60 mg/kg | 1 mg/kg |
| Days | PO, QD | PO, QD | PO, QD | PO, QD | PO, QOD | PO, QD |
| 0 | 154 ± 11 | 154 ± 12 | 155 ± 11 | 156 ± 12 | 154 ± 11 | 154 ± 12 |
| 3 | 240 ± 18 | 238 ± 16 | 237 ± 22 | 220 ± 17 | 233 ± 20 | 239 ± 18 |
| 7 | 314 ± 27 | 277 ± 20 | 273 ± 26 | 264 ± 14 | 304 ± 27 | 306 ± 17 |
| 10 | 339 ± 33 | 291 ± 28 | 241 ± 25 | 227 ± 10 | 273 ± 28 | 346 ± 22 |
| 14 | 353 ± 36 | 275 ± 28 | 230 ± 19 | 202 ± 10 | 234 ± 22 | 358 ± 22 |
| 17 | 396 ± 40 | 281 ± 34 | 214 ± 16 | 174 ± 12 | 215 ± 22 | 395 ± 30 |
| 21 | 444 ± 55 | 269 ± 43 | 193 ± 17 | 160 ± 10 | 193 ± 20 | 411 ± 31 |
| 24 | 498 ± 74 | 270 ± 40 | 174 ± 19 | 156 ± 8 | 183 ± 21 | 423 ± 35 |
| 28 | 631 ± 112 | 273 ± 39 | 172 ± 18 | 156 ± 8 | 177 ± 22 | 473 ± 44 |

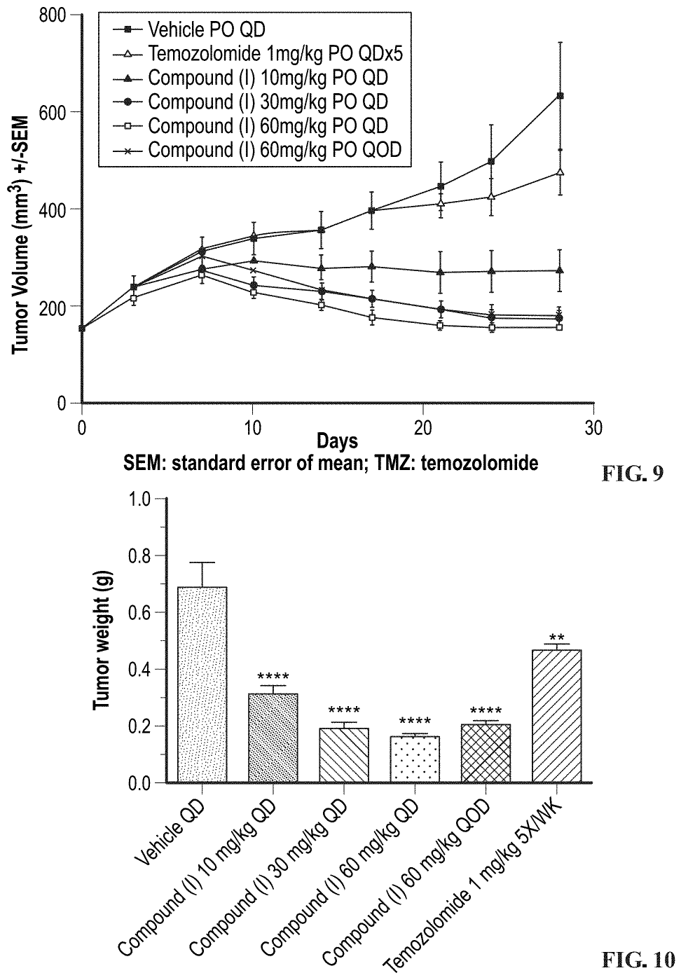

The inhibition rates of the compound of formula (I) and temozolomide are calculated with tumor size data on PG-D27. The calculation results are shown in Table 31. Tumor weight graphs are shown in FIG. 10.

TABLE 31
Tumor Growth Inhibition Analysis

| | | Tumor Size | TGI | T/C | |
|---|---|---|---|---|---|
| Group | Treatment | (mm³) | (%) | (%) | p value |
| 1 | Vehicle | 631 ± 112 | — | — | — |
| 2 | Compound (I) | 273 ± 39 | 75.08 | 43.20 | **** |
| | 10 mg/kg PO, QD | | | | |
| 3 | Compound (I) | 172 ± 18 | 96.40 | 27.32 | **** |
| | 30 mg/kg PO, QD | | | | |
| 4 | Compound (I) | 156 ± 8 | 100.03 | 24.64 | **** |
| | 60 mg/kg PO, QD | | | | |
| 5 | Compound (I) | 177 ± 22 | 95.14 | 28.09 | **** |
| | 60 mg/kg PO, QOD | | | | |
| 6 | Temozolomide 1 | 473 ± 44 | 33.24 | 74.94 | ns |
| | mg/kg PO, QD | | | | |

Note:
**** p < 0.0001,
ns p > 0.05 vs vehicle.

8.6 Discussion

The results of this study show that relative to the control group, the compound of formula (I) demonstrated dose-dependent and statistically significant antitumor activity. On the other hand, temozolomide did not demonstrate statistically significant antitumor activity relative to the control group. In addition, the results show that the effect of compound (I) dosed at 30 mg/kg QD was similar to that of compound (I) dosed at 60 mg/kg QOD, suggesting that daily dosing or less frequent dosing, such as dosing every other day, may be possible.

### Example 9: Evaluation of the Compound of Formula (I) in Enzalutamide-Resistant Patient-Derived Xenograft Prostate Model

Tumor fragments from a mCRPC patient were implanted subcutaneously into the flank of mice. After tumors reached an average of 150-300 mm³, mice (n=10/group) received oral QD administration by oral gavage of vehicle, enzalutamide (30 mg/kg), the compound of formula (I) (30 mg/kg) or the combination of the compound of formula (I)+enzalutamide. Tumor volumes and Animal body weights were also measured twice weekly. Tumor size was measured twice weekly in two dimensions using a caliper, and the volume was expressed in mm³ using the formula: V=0.5 a×b² where a and b are the long and short diameters of the tumor, respectively. The tumor sizes were then used for the calculation of T/C (tumor/control) and TGI (tumor growth inhibition) values. The T/C value (in percent) was calculated where T and C are the mean volume of the treated and control groups, respectively, on a given day. TGI was calculated for each group using the formula: TGI (%)=[1−(Ti−T0)/(Vi−V0)]×100; Ti is the average tumor volume of a treatment group on PG-D29, T0 is the average tumor volume of the treatment group on the day of treatment start, Vi is the average tumor volume of the vehicle control group on the same day with Ti, and V0 is the average tumor volume of the vehicle group on the day of treatment start. All the mice were taken down on day 30 when the tumor volume of the vehicle treated group reached ˜1500-2000 mm³.

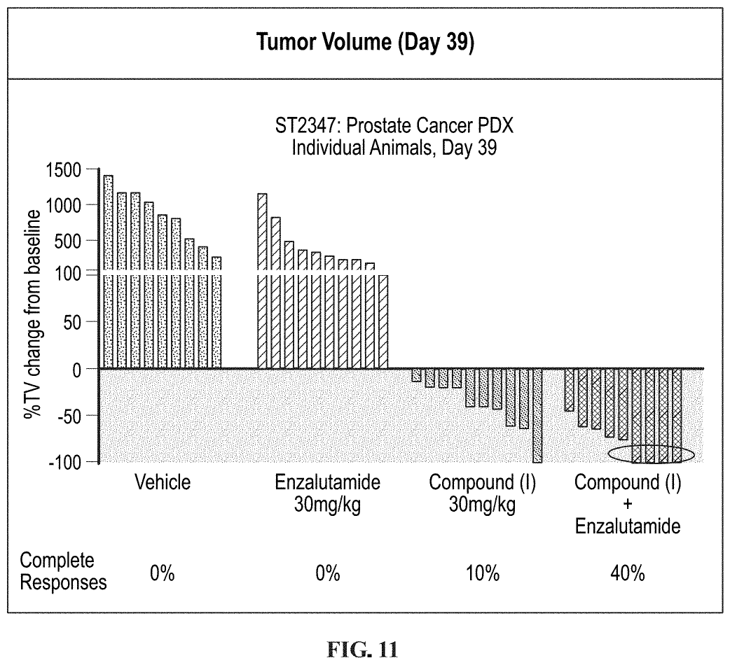

In this study, the antitumor effect of compound (I), enzalutamide, or combinations thereof on tumors in mice that were derived from tumor fragments from an mCRPC (metastatic castration-resistant prostate cancer) human patient were evaluated. The results from the study are shown in FIG. 11. The compound of formula (I) demonstrated statistically significant antitumor activity over the course of the treatment whereas enzalutamide had minimal effect. However, enzalutamide significantly enhanced the efficacy of the compound of formula (I) when the two compound were administered in combination. The compound of formula (I) resulted in tumor regression in all cases, with >50% reduction in tumor volume in 3 of 10 mice in the group at day 39 of treatment, including one mouse with 100% reduction in tumor volume. The combination of the compound of formula (I) and enzalutamide resulted in >50% reduction in tumor volume in 8 of 9 mice in the group at day 39 of treatment, including four mice with 100% reduction in tumor volume.

### Example 10: Evaluation of the Compound of Formula (I) in a HER2+Xenograft Model-Monotherapy or Combination with One or More HER2 Inhibitors

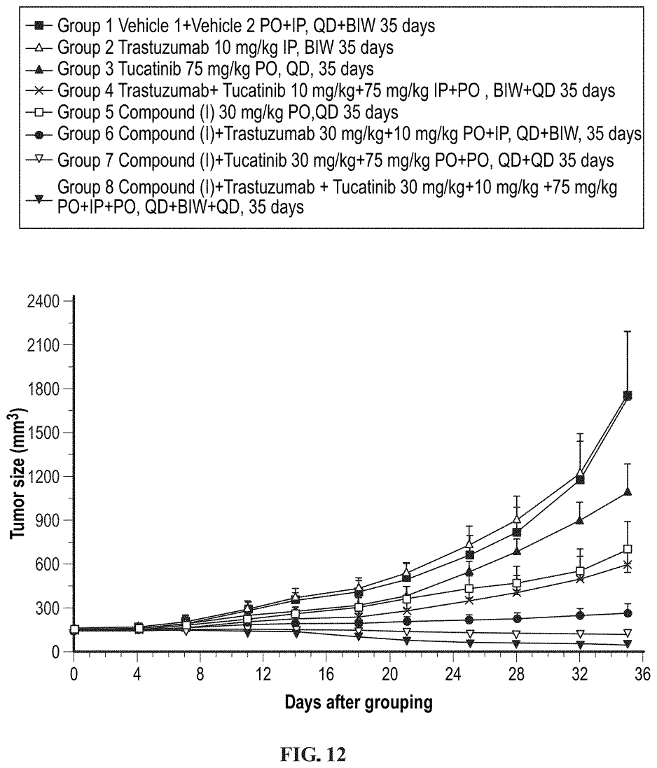

The HCC1954 tumor cells were maintained in vitro in medium supplemented with 10% heat inactivated fetal bovine serum at 37° C. in an atmosphere of 5% CO2 in air. The tumor cells were routinely subcultured twice weekly. The cells growing in an exponential growth phase were harvested and counted for tumor inoculation. Each mouse was inoculated subcutaneously at the right flank with HCC1954 tumor cells (10×10⁶) in 0.2 mL of PBS mixed with Matrigel (50:50) for tumor development. The treatments were started on day 9 after tumor inoculation when the average tumor volume reached 150 mm³. The test articles or vehicle control were administrated to the mice according to the group assignment. Mice (n=8/group) received vehicle, trastuzumab (10 mg/kg, IP, BIW), tucatinib (75 mg/kg, PO, QD), compound of formula (I) (30 mg/kg, PO, QD) as single agents or in combination (the compound of formula (I)+trastuzumab, the compound of formula (I)+tucatinib, trastuzumab+tucatinib, or all three agents the compound of formula (I)+tucatinib+trastuzumab). Animal body weights were also measured twice weekly. Tumor size was measured twice weekly in two dimensions using a caliper, and the volume was expressed in mm³ using the formula: V=0.5 a×b² where a and b are the long and short diameters of the tumor, respectively. The tumor sizes will then be used for the calculation of T/C (tumor/control) and TGI (tumor growth inhibition) values. The T/C value (in percent) was calculated where T and C are the mean volume of the treated and control groups, respectively, on a given day. TGI was calculated for each group using the formula: TGI (%)=[1−(Ti−T0)/(Vi−V0)]×100; Ti is the average tumor volume of a treatment group during takedown day, T0 is the average tumor volume of the treatment group on the day of treatment start, Vi is the average tumor volume of the vehicle control group on the same day with Ti, and V0 is the average tumor volume of the vehicle group on the day of treatment start. The results are shown in FIG. 12.

In this study, the antitumor effect of the compound of formula (I), trastuzumab, and tucatinib, and combinations thereof, on HCC1954 tumors in mice was evaluated. All treatments, with the exception of trastuzumab monotherapy, resulted in tumor growth inhibition. Treatment with a dual combination of compound (I) (30 mg/kg PO QD) and trastuzumab (10 mg/kg IP BIW) substantially inhibited tumor growth. The dual combination of the compound of formula (I) (30 mg/kg PO QD) and tucatinib (75 mg/kg PO QD), and the triple combination of compound of formula (I) (30 mg/kg PO QD), tucatinib (75 mg/kg PO QD), and trastuzumab (10 mg/kg IP BIW), resulted in apparent tumor regression.

### Example 11: A Phase ½ Dose Escalation, Safety, Pharmacokinetics, and Efficacy Study of the Compound of Formula (I) in Adults with Recurrent or Refractory High-Grade Gliomas and Solid Tumors

11.1 Study Description

A Phase ½ dose escalation and multiple expansion cohort study designed to evaluate the safety and efficacy of the compound of formula (I) will be performed. The study population is comprised of adults with recurrent or refractory high-grade gliomas (HGGs), metastatic breast cancer (mBC), with and without brain metastases, and recurrent or refractory metastatic castration-resistant prostate cancer (mCRPC). All patients will self-administer the compound of formula (I) orally in 28-day cycles until disease progression, toxicity, withdrawal of consent, or termination of the study.

11.2 Conditions Evaluated
Gliomoa
Glioma, malignant
Glioma, mixed
Glial cell tissues
Breast Cancer
Breast Carcinoma
Cancer of Breast
Cancer of the Breast
Breast Tumor
Malignant Tumor of the Breast
Prostate Cancer
Prostatic Cancer
Cancer of Prostate
Cancer of the Prostate
Prostate Neoplasm
Malignant Glioma
Metastatic Breast Cancer
Metastatic Castration-Resistant Prostate Cancer

11.3 Study Design
Study Type: Interventional Primary Purpose: Treatment
Study Phase: Phase 1/Phase 2 Interventional Study Model: Sequential Assignment
Number of Arms: 2
Masking: None (Open Label)
Allocation: Non-Randomized
Enrollment: 218 [Anticipated]

11.4 Experimental Arms
Experimental: Phase 1 Dose Escalation of the compound of formula (I) administered at escalating dose levels until the maximum tolerated dose (MTD) is reached
Experimental: The compound of formula (I) administered at the recommended Phase 2dose (RP2D)

11.5 Primary Outcome Measures
1. Phase 1 Dose Escalation
   Incidence of treatment-emergent adverse events (TEAEs), serious adverse events (SAEs), and dose-limiting toxicities (DLTs)
   [Time Frame: During the DLT period (28 days)]
2. Phase 2 Dose Expansion Cohort 1
   Objective response rate (ORR) and Duration of Response (DOR)
   [Time Frame: Every 8 weeks through study treatment, an average of 6 months]
3. Phase 2 Dose Expansion Cohort 2
   Evaluation of the compound of formula (I) concentration in plasma, including peak plasma concentration
   [Time Frame: Intraoperatively after 21 days of study treatment]
4. Phase 2 Dose Expansion Cohort 2
   Evaluation of the compound of formula (I) half-life in plasma
   [Time Frame: Intraoperatively after 21 days of study treatment]
5. Phase 2 Dose Expansion Cohort 2
   Evaluation of compound of formula (I) concentration in brain tumor tissue
   [Time Frame: Intraoperatively after 21 days of study treatment]
6. Phase 2 Dose Expansion Cohort 2
   Evaluation of the compound of formula (I) effects on brain tumor cell proliferation ratio pre- and post-treatment
   [Time Frame: Intraoperatively after 21 days of study treatment]
7. Phase 2 Dose Expansion Cohort 3
   ORR
   [Time Frame: Every 8 weeks through study treatment, an average of 6 months]
8. Phase 2 Dose Expansion Cohort 3
   DOR
   [Time Frame: Every 8 weeks through study treatment, an average of 6 months]
9. Phase 2 Dose Expansion Cohort 4
   OPR
   [Time Frame: Every 8 weeks through study treatment, an average of 6 months]
10. Phase 2 Dose Expansion Cohort 4
   DOR
   [Time Frame: Every 8 weeks through study treatment, an average of 6 months]
11. Phase 2 Dose Expansion Cohort 4
   Decease in the prostate-specific antigen (PSA) pre- and post-treatment
   [Time Frame: Every 4 weeks through study treatment, an average of 6 months]
12. Phase 2 Dose Expansion Cohort 5
   OPR
   [Time Frame: Every 8 weeks through study treatment, an average of 6 months]
13. Phase 2 Dose Expansion Cohort 5
   DOR
   [Time Frame: Every 8 weeks through study treatment, an average of 6 months]

11.6 Eligibility

Eligible individuals must meet all the following criteria:
1. Minimum Age: 18 years
2. Sex: All
3. Gender Based: No
4. Accepts Healthy Volunteers: No

11.6.1 Criteria for all Phases and Cohorts
1. Recovered from toxicity to prior anti-cancer therapy
2. Adequate bone marrow and organ function
3. Appropriate candidate for monotherapy with the compound of formula (I)
4. Life expectancy of >3 months

11.6.2 Cohort-Specific Inclusion Criteria

11.6.2.1 Phase 1 High Grade Glioma
1. Histologically confirmed diagnosis of high-grade glioma
2. Evidence of recurrence after treatment (i.e., surgery, radiation, or temozolomide [TMZ]) or refractory (or intolerant) to treatment after ≤2 prior lines of therapy
3. Measurable or non-measurable disease
4. Karnofsky Performance Status (KPS) score ≥60

11.6.2.2 Phase 1 HR+HER2− mBC
1. Men and women who are not suitable for surgical resection or radiotherapy for the purpose of cure
2. Diagnosis of locally advanced or HR+HER2 metastatic breast cancer
3. Evidence of progression as determined by the Investigator per standard criteria
4. Patients must have endocrine-resistant disease
5. Have no known active or symptomatic central nervous system (CNS) disease
6. Eastern Cooperative Oncology Group Performance Status (ECOG PS)≤2

11.6.2.3 Phase 1 mCRPC
1. Diagnosis of metastatic castration-resistant prostate cancer with disease progression despite castrate levels of testosterone
2. Have radiographic or biochemical evidence of progression as determined by the Investigator per standard criteria
3. Have no known active or symptomatic CNS disease
4. Received prior therapy with anti-androgen(s) and taxane-based chemotherapy for castration-resistant disease
5. ECOG PS≤2

11.6.2.4 Phase 2 Expansion Cohort 1—(Glioblastoma)
1. Histologically confirmed diagnosis of glioblastoma
2. Received prior therapy with radiation or radiation plus temozolomide
3. Radiographic evidence of progression and measurable disease as determined by the investigator per standard criteria.
4. KPS score ≥70

11.6.2.5 Phase 2 Expansion Cohort 2—(Glioblastoma)
1. Histologically confirmed diagnosis of glioblastoma
2. Received prior therapy with radiation or radiation plus temozolomide
3. Radiographic evidence of progression and measurable disease as determined by the investigator per standard criteria
4. KPS score ≥70
5. Eligible for surgical resection

11.6.2.6 Phase 2 Expansion Cohort 3 HR+HER2− mBC
1. Men and women who are not suitable for surgical resection or radiotherapy for the purpose of cure
2. Diagnosis of locally advanced or HR+HER2− metastatic breast cancer
3. Evidence of progression and measurable disease as determined by the investigator per standard criteria
4. Have no known active or symptomatic CNS disease
5. ECOG PS≤2

11.6.2.7 Phase 2 Expansion Cohort 4 mCRPC
1. Diagnosis of metastatic castration-resistant prostate cancer with disease progression despite castrate levels of testosterone
2. Have radiographic or biochemical evidence of progression and measurable disease as determined by the investigator per standard criteria
3. Have no known active or symptomatic CNS disease
4. Received prior therapy with anti-androgen(s) and taxane-based chemotherapy for castration-resistant disease
5. ECOG PS≤2

11.6.2.8 Phase 2 Expansion Cohort 5 HR+HER2− mBC with Brain Metastases
1. Men and women who are not suitable for surgical resection or radiotherapy for the purpose of cure
2. Diagnosis of HR+HER2− metastatic breast cancer with brain lesion(s)
3. Evidence of progression and measurable disease as determined by the Investigator per standard criteria
4. ECOG PS≤2

11.7 Key Exclusion Criteria for all Phases and Cohorts
1. Have received chemotherapy, hormonal therapy (with the exception of ongoing LHRH analogs in male patients and premenopausal women), radiation, or biological anti-cancer therapy within 14 days prior to the first dose of the compound of formula (I)
2. Has a history of or current use of bevacizumab (glioma and brain metastases only)
3. Received treatment with an investigational agent for any indication within 14 days for non-myelosuppressive agent or 21 days (or <5 half-lives) for myelosuppressive agent prior to the first dose of the compound of formula (I)
4. Requires systemic corticosteroid therapy >4 mg/day (>2 mg/day for Expansion Cohort 2) of dexamethasone or equivalent or increasing doses of systemic corticosteroids during the 7 days prior to enrollment
5. Requires anti-seizure medications that are known to be strong inducers of CYP3A4/5 enzymes (carbamazepine, phenytoin) or has a recent history of uncontrolled or intermittent seizures
6. Females who are pregnant or breast feeding

### Example 12: In Vivo Efficacy Evaluation in a Patient-Derived Xenograft (PDX) Model Representing Human Breast Cancer in Immune-Deficient Mice

Compound (I), fulvestrant, RAD1901 (elacestrant), and palbociclib were tested in a breast cancer PDX model harboring ESR1^{Y537S} mutation. The study outline is provided in Table 32 below.

TABLE 32

| | | | Dose | ROA/ | Total | Treatment |
|---|---|---|---|---|---|---|
| Group | N | Treatment | (mg/kg) | Schedule | Dosed | Day(s) |
| 1 | 8 | Vehicle | — | PO/qd to | 33 | 0-32 |
| | | | | end | | |
| 2 | 8 | Fulvestrant | *3 | SC/q7d to | 7 | 0, 7, 14, |
| | | | | end | | 21, 28 |
| 3 | 8 | RAD1901 | 30 | PO/qd to | 47 | 0-32 |
| | | | | end | | |
| 4 | 8 | Palbociclib | 25 | PO/qd to | 47 | 0-32 |
| | | | | end | | |
| 5 | 8 | Compound (I) | 30 | PO/qd to | 47 | 0-32 |
| | | | | end | | |
| 6 | 8 | Compound (I) | 30 | PO/qd to | 47 | 0-32 |
| | | | | end | | |
| | | Fulvestrant | *3 | SC/q7d to | 7 | 0, 7, 14, |
| | | | | end | | 21, 28 |
| 7 | 8 | Compound (I) | 30 | PO/qd to | 47 | 0-32 |
| | | | | end | | |
| | | RAD1901 | 30 | PO/qd to | 47 | 0-32 |
| | | | | end | | |

*Administered at a fixed-volume (mg/dose)

Tumor volume data is provided in Table 33 below.

TABLE 33
Tumor Volume (mm³, Mean ± SEM)

| Group | 1 | 2 | 3 | 4 | 5 | 6 | 7 |
|---|---|---|---|---|---|---|---|
| Day 0 | 124 ± 3 | 124 ± 3 | 124 ± 3 | 124 ± 3 | 124 ± 3 | 122 ± 4 | 122 ± 4 |
| Day 4 | 158 ± 11 | 162 ± 14 | 123 ± 10 | 146 ± 11 | 115 ± 9 | 106 ± 9 | 106 ± 9 |
| Day 6 | 218 ± 18 | 199 ± 22 | 138 ± 16 | 200 ± 25 | 120 ± 8 | 112 ± 12 | 69 ± 4 |
| Day 11 | 565 ± 50 | 511 ± 62 | 199 ± 28 | 447 ± 57 | 102 ± 12 | 115 ± 6 | 58 ± 5 |
| Day 14 | 901 ± 103 | 802 ± 123 | 305 ± 45 | 632 ± 65 | 137 ± 24 | 146 ± 16 | 45 ± 4 |
| Day 18 | 1268 ± 155 | 1036 ± 131 | 382 ± 58 | 914 ± 69 | 173 ± 23 | 156 ± 14 | 45 ± 4 |
| Day 21 | 1675 ± 182 | 1254 ± 164 | 482 ± 78 | 1161 ± 122 | 181 ± 24 | 193 ± 27 | 49 ± 5 |
| Day 26 | 2210 ± 302 | 1577 ± 198 | 574 ± 76 | 1551 ± 188 | 199 ± 25 | 208 ± 30 | 40 ± 5 |
| Day 29 | 2425 ± 267 | 1808 ± 195 | 696 ± 100 | 1667 ± 185 | 237 ± 30 | 221 ± 37 | 31 ± 3 |
| Day 32 | | 1809 ± 180 | 801 ± 111 | | 245 ± 28 | 231 ± 37 | 27 ± 4 |

In this study, the antitumor effect of the compound of formula (I) (30 mg/kg PO QD), fulvestrant (3 mg/dose SC, q7d), RAD1901 (elacestrant) (30 mg/kg PO QD), and palbociclib (25 mg/kg PO QD), and combinations thereof, on a breast cancer PDX model harboring ESR1^{Y537S} mutation was evaluated. The vehicle control group reached a tumor size of 2425 mm³. All treatment groups inhibited tumor growth, with compound of formula (I) monotherapy providing strong inhibition (245 mm³). The combination of the compound of formula (I) and fulvestrant (231 mm³ tumor volume) provided a comparable effect to that of the compound of formula (I) alone. The combination of the compound of formula (I) and RAD1901 resulted in tumor regression from day 4 through the end of the study, with near complete tumor regression by the end of the study (27 mm³ tumor volume).

### Example 13: In Vivo Efficacy Study in xMDA-MB-361 Breast Tumor Model in Female BALB/c Nude Mice

Trastuzumab, tucatinib, Compound (I), and fulvestrant were tested in a xMDA-MB-361 breast tumor model (ER-positive/HER2-positive) on female BALB/c nude mice. The study outline is provided in Table 34 below.

TABLE 34

| | | | | | Dosing | | | |
|---|---|---|---|---|---|---|---|---|
| | | | Dose | | Volume | | Dosing | |
| Group | N | Treatment | (mg/kg) | | (μl/g) | | Route | Schedule |
| 1 | 8 | Vehicle | — | | 10 | μl/g | PO | QD*28 days |
| 2 | 8 | Fulvestrant | 1 | mg/dose | 20 | μl/mouse | SC | QW*42 days |
| 3 | 8 | Tucatinib | 60 | mg/kg | 10 | μl/g | PO | QD*42 days |
| 4 | 8 | Fulvestrant + | 1 mg/dose + | | 20 μl/mouse + | | SC + PO | QW + QD*42 |
| | | Tucatinib | 60 mg/kg | | 10 μl/g | | | days |
| 5 | 8 | Compound (I) | 30 | mg/kg | 10 | μl/g | PO | QD*42 days |

| | | | | | | | |
|---|---|---|---|---|---|---|---|
| 6 | 8 | Compound (I) + | 30 mg/kg + | 10 | μl/g | PO + PO | QD + QD*42 |
| | | Tucatinib | 60 mg/kg | | | | days |

| | | | | | | |
|---|---|---|---|---|---|---|
| 7 | 8 | Compound (I) + | 30 mg/kg + | 10 μl/g + | PO + | QD + QW + |
| | | Fulvestrant + | 1 mg/dose + | 20 μl/mouse + | SC + PO | QD*42 days |
| | | Tucatinib | 60 mg/kg | 10 μl/g | | |
| 8 | 8 | Compound (I) + | 10 mg/kg + | 10 μl/g + | PO + | QD + QW + |
| | | Fulvestrant + | 1 mg/dose + | 20 μl/mouse + | SC + PO | QD*42 days |
| | | Tucatinib | 1 mg/kg | 10 μl/g | | |
| 9 | 8 | Compound (I) + | 30 mg/kg + | 10 μl/g + | PO + | QD + QW + |
| | | Fulvestrant + | 1 mg/dose + | 20 μl/mouse + | SC + IP | QW*42 days |
| | | Trastuzumab | 10 mg/kg | 10 μl/g | | |

Tumor volume data is provided in Table 35 below.

TABLE 35
Tumor Volume (mm³, Mean ± SEM)

| Group | 1 | 2 | 3 | 4 | 5 | 6 | 7 | 8 | 9 |
|---|---|---|---|---|---|---|---|---|---|
| Day 0 | 157 ± 12 | 157 ± 13 | 157 ± 11 | 157 ± 13 | 157 ± 11 | 157 ± 11 | 157 ± 11 | 157 ± 12 | 157 ± 11 |
| Day 3 | 205 ± 13 | 185 ± 11 | 148 ± 11 | 195 ± 12 | 202 ± 10 | 195 ± 13 | 141 ± 11 | 142 ± 11 | 147 ± 9 |
| Day 7 | 304 ± 26 | 226 ± 28 | 156 ± 20 | 195 ± 18 | 271 ± 34 | 228 ± 17 | 104 ± 12 | 126 ± 13 | 155 ± 10 |
| Day 10 | 422 ± 67 | 321 ± 53 | 219 ± 30 | 235 ± 31 | 316 ± 43 | 240 ± 20 | 89 ± 12 | 122 ± 17 | 145 ± 7 |
| Day 14 | 631 ± 96 | 489 ± 101 | 408 ± 59 | 409 ± 75 | 342 ± 49 | 256 ± 38 | 80 ± 12 | 113 ± 25 | 93 ± 7 |
| Day 17 | 919 ± 167 | 588 ± 153 | 658 ± 112 | 596 ± 145 | 326 ± 70 | 202 ± 42 | 55 ± 14 | 118 ± 23 | 81 ± 20 |
| Day 21 | 1318 ± 213 | 773 ± 204 | 970 ± 206 | 889 ± 227 | 360 ± 84 | 200 ± 45 | 50 ± 13 | 126 ± 28 | 42 ± 12 |
| Day 24 | 1549 ± 249 | 890 ± 235 | 1185 ± 222 | 1090 ± 316 | 429 ± 119 | 189 ± 40 | 44 ± 15 | 107 ± 25 | 28 ± 9 |
| Day 28 | 1914 ± 314 | 1022 ± 256 | 1496 ± 250 | 1247 ± 348 | 542 ± 146 | 193 ± 44 | 39 ± 12 | 136 ± 45 | 22 ± 11 |
| Day 31 | — | 1113 ± 293 | 1707 ± 309 | 994 ± 187 | 571 ± 150 | 182 ± 47 | 28 ± 8 | 117 ± 52 | 16 ± 9 |
| Day 35 | — | 1192 ± 330 | 1580 ± 240 | 1019 ± 201 | 587 ± 148 | 164 ± 47 | 21 ± 6 | 92 ± 42 | 16 ± 10 |
| Day 38 | — | 1300 ± 344 | 1675 ± 283 | 847 ± 171 | 629 ± 164 | 129 ± 49 | 18 ± 5 | 86 ± 42 | 14 ± 7 |
| Day 42 | — | 1396 ± 356 | 1670 ± 344 | 803 ± 156 | 608 ± 145 | 111 ± 36 | 16 ± 4 | 70 ± 32 | 9 ± 5 |

In this study, the antitumor effect of the compound of formula (I) (10 mg/kg or 30 mg/kg PO QD), fulvestrant (1 mg/dose SC, QW), tucatinib (1 mg/kg or 60 mg/kg PO QD), and trastuzumab (10 mg/kg IP QW), and combinations thereof, on xMDA-MB-361 breast tumor model in female BALB/c nude mice was evaluated. The vehicle control group reached a tumor size of 1914 mm³. All treatment groups inhibited tumor growth. The dual combination of compound (I) (30 mg/kg) and tucatinib (60 mg/kg) provided strong inhibition (111 mm³ tumor volume at the end of the study). Triple combinations of Compound (I) (30 mg/kg), fulvestrant (1 mg/dose), and tucatinib (60 mg/kg), as well as compound (I) (30 mg/kg), fulvestrant (1 mg/dose), and trastuzumab (10 mg/kg), resulted in tumor regression from treatment day 3 through the end of the study, with near complete tumor regression by the end of the study (16 and 9 mm³ tumor volumes, respectively).

### Example 14: In Vivo Efficacy Study in xBT474 Breast Tumor Model in Female BALB/c Nude Mice

Trastuzumab, Tucatinib, Compound (I) and Fulvestrant were tested in xBT474 (ER-negative/HER2-positive) model in female BALB/c nude mice. The study outline is provided in Table 36 below.

TABLE 36

| | | | | | Dosing | | | |
|---|---|---|---|---|---|---|---|---|
| | | | Dose | | Volume | | Dosing | |
| Group | N | Treatment | (mg/kg) | | (μl/g) | | route | Schedule |
| 1 | 8 | Vehicle | — | | 10 | μl/g | PO | QD*28 days |
| 2 | 8 | Fulvestrant | 1 | mg/dose | 20 | μl/mouse | SC | QW*28 days |
| 3 | 8 | Tucatinib | 25 | mg/kg | 10 | μl/g | PO | QD*28 days |
| 4 | 8 | Fulvestrant + | 1 mg/dose + | | 20 μl/mouse + | | SC + PO | QW + QD*28 |
| | | Tucatinib | 25 mg/kg | | 10 μl/g | | | days |
| 5 | 8 | Compound (I) | 30 | mg/kg | 10 | μl/g | PO | QD*28 days |

| | | | | | | | |
|---|---|---|---|---|---|---|---|
| 6 | 8 | Compound (I) + | 30 mg/kg + | 10 | μl/g | PO + PO | QD + QD*28 |
| | | Tucatinib | 25 mg/kg | | | | days |

| | | | | | | |
|---|---|---|---|---|---|---|
| 7 | 8 | Compound (I) + | 30 mg/kg + | 10 μl/g + | PO + SC + IP | QD + QW + QW |
| | | Fulvestrant + | 1 mg/dose + | 20 μl/mouse + | | *28 days |
| | | Trastuzumab | 1 mg/kg | 10 μl/g | | |
| 8 | 8 | Compound (I) + | 30 mg/kg + | 10 μl/g + | PO + SC + PO | QD + QW + QD |
| | | Fulvestrant + | 1 mg/dose + | 20 μl/mouse + | | *28 days |
| | | Tucatinib | 25 mg/kg | 10 μl/g | | |
| 9 | 8 | Compound (I) + | 10 mg/kg + | 10 μl/g + | PO + SC + PO | QD + QW + QD |
| | | Fulvestrant + | 1 mg/dose + | 20 μl/mouse + | | *28 days |
| | | Tucatinib | 1 mg/kg | 10 μl/g | | |

Tumor volume data is provided in Table 37 below.

TABLE 37
Tumor Volume (mm³, Mean ± SEM)

| Group | 1 | 2 | 3 | 4 | 5 | 6 | 7 | 8 | 9 |
|---|---|---|---|---|---|---|---|---|---|
| Day 0 | 146 ± 10 | 146 ± 10 | 146 ± 10 | 146 ± 9 | 146 ± 8 | 146 ± 10 | 146 ± 10 | 146 ± 11 | 146 ± 12 |
| Day 4 | 252 ± 22 | 188 ± 26 | 177 ± 11 | 190 ± 15 | 240 ± 20 | 193 ± 11 | 188 ± 11 | 157 ± 11 | 143 ± 10 |
| Day 7 | 468 ± 70 | 187 ± 14 | 199 ± 19 | 213 ± 14 | 250 ± 22 | 114 ± 9 | 207 ± 28 | 112 ± 11 | 109 ± 13 |
| Day 11 | 728 ± 121 | 311 ± 55 | 168 ± 16 | 262 ± 41 | 347 ± 45 | 96 ± 13 | 270 ± 32 | 109 ± 10 | 107 ± 16 |
| Day 14 | 967 ± 173 | 391 ± 55 | 169 ± 15 | 297 ± 43 | 408 ± 42 | 94 ± 11 | 307 ± 37 | 87 ± 7 | 82 ± 10 |
| Day 18 | 1245 ± 230 | 468 ± 69 | 234 ± 28 | 318 ± 46 | 490 ± 59 | 73 ± 10 | 311 ± 41 | 57 ± 4 | 65 ± 7 |
| Day 21 | 1476 ± 276 | 532 ± 79 | 288 ± 35 | 340 ± 52 | 549 ± 73 | 63 ± 8 | 326 ± 43 | 45 ± 4 | 60 ± 7 |
| Day 25 | 1499 ± 201 | 617 ± 93 | 341 ± 47 | 326 ± 48 | 576 ± 79 | 57 ± 8 | 335 ± 49 | 38 ± 3 | 55 ± 7 |
| Day 28 | 1728 ± 216 | 741 ± 113 | 397 ± 68 | 320 ± 49 | 617 ± 84 | 45 ± 8 | 367 ± 52 | 27 ± 2 | 52 ± 9 |
| Day 32 | — | 750 ± 115 | 406 ± 96 | 347 ± 44 | 658 ± 99 | 39 ± 7 | 366 ± 58 | 30 ± 3 | 62 ± 12 |
| Day 35 | — | 797 ± 121 | 488 ± 112 | 415 ± 46 | 721 ± 119 | 39 ± 7 | 406 ± 64 | 30 ± 4 | 86 ± 21 |
| Day 39 | — | 800 ± 131 | 420 ± 91 | 403 ± 59 | 685 ± 107 | 48 ± 11 | 444 ± 75 | 32 ± 5 | 100 ± 23 |
| Day 42 | — | 843 ± 146 | 431 ± 89 | 404 ± 64 | 694 ± 120 | 64 ± 17 | 439 ± 83 | 40 ± 8 | 123 ± 25 |
| Day 46 | — | 816 ± 138 | 421 ± 81 | 418 ± 64 | 707 ± 116 | 82 ± 24 | 500 ± 101 | 52 ± 10 | 145 ± 25 |

In this study, the antitumor effect of the compound of formula (I) (10 mg/kg or 30 mg/kg PO QD), fulvestrant (1 mg/dose SC, QW), tucatinib (1 mg/kg or 25 mg/kg PO QD), and trastuzumab (1 mg/kg IP QW), and combinations thereof, on xBT474 breast tumor model in female BALB/c nude mice was evaluated. The vehicle control group reached a tumor size of 1728 mm³. All treatment groups inhibited tumor growth. The dual combination of compound (I) (30 mg/kg) and tucatinib (25 mg/kg), and the triple combination of Compound (I) (30 mg/kg), fulvestrant (1 mg/dose), and tucatinib (25 mg/kg), provided strong inhibition (82 mm³ and 52 mm³ tumor volumes, respectively), with tumor regression from treatment day 7 through the end of the study.

### Example 15: In Vivo Efficacy Evaluation in Non-Castrated VCap Prostate Cancer Model in Male CB17/Scid Mice

Compound (I), enzalutamide, and abiraterone were tested in non-castrated VCap prostate cancer model in male CB17/Scid mice. The study outline is provided in Table 38 below.

TABLE 38

| | | | Dose | Dosing | | | |
|---|---|---|---|---|---|---|---|
| | | | level | Solution | DosingVolume | | |
| Group | N | Treatment | (mg/kg) | (mg/ml) | (μL/g) | ROA | Schedule |
| 1 | 8 | Vehicle | — | — | 10 | p.o. | QD * 26 days |
| 2 | 8 | Enzalutamide | 20 | 2 | 10 | p.o. | QD * 26 days |
| 3 | 8 | Compound (I) | 10 | 1 | 10 | p.o. | QD * 26 days |
| 4 | 8 | Compound (I) | 30 | 3 | 10 | p.o. | QD * 26 days |
| 5 | 8 | Enzalutamide | 20 | 2 | 10 | p.o. | QD * 26 days |
| | | Compound (I) | 10 | 1 | 10 | p.o. | QD * 26 days |
| 6 | 8 | Enzalutamide | 20 | 2 | 10 | p.o. | QD * 26 days |
| | | Compound (I) | 30 | 3 | 10 | p.o. | QD * 26 days |
| 7 | 8 | Abiraterone | 350 | 35 | 10 | p.o. | QD * 26 days |
| 8 | 8 | Abiraterone | 350 | 35 | 10 | p.o. | QD * 26 days |
| | | Compound (I) | 10 | 1 | 10 | p.o. | QD * 26 days |
| 9 | 8 | Abiraterone | 350 | 35 | 10 | p.o. | QD * 26 days |
| | | Compound (I) | 30 | 3 | 10 | p.o. | QD * 26 days |

Tumor volume data is provided in Table 39 below.

TABLE 39
Tumor Volume (mm³, Mean ± SEM)

| Group | 1 | 2 | 3 | 4 | 5 | 6 | 7 | 8 | 9 |
|---|---|---|---|---|---|---|---|---|---|
| Day 0 | 171.7 ± 5.9 | 171.8 ± 6.1 | 171.8 ± 6 | 171.7 ± 6.1 | 171.8 ± 5.9 | 171.8 ± 5.9 | 171.7 ± 5.9 | 171.8 ± 6.1 | 171.6 ± 6 |
| Day 4 | 312.4 ± 18.4 | 287.4 ± 17 | 221.9 ± 15.2 | 218.9 ± 14.4 | 240 ± 16 | 222.9 ± 16.9 | 278 ± 22.9 | 238.3 ± 19 | 242.5 ± 16.2 |
| Day 7 | 403.9 ± 18.3 | 352.2 ± 28.8 | 260.9 ± 13.8 | 233.4 ± 16.4 | 255.5 ± 19.9 | 224.6 ± 19.6 | 365.9 ± 35.9 | 289.6 ± 19.4 | 275.3 ± 17.3 |
| Day 11 | 618.3 ± 35.8 | 542.8 ± 50.7 | 310.5 ± 19.9 | 253.7 ± 20.6 | 305.7 ± 34.6 | 258.8 ± 26.1 | 582.6 ± 43.3 | 372 ± 23.9 | 306.2 ± 18.2 |
| Day 14 | 808.8 ± 72.5 | 662.7 ± 51.7 | 381.2 ± 15.4 | 279.1 ± 23.6 | 351.1 ± 25.2 | 288.2 ± 26 | 762.2 ± 73.7 | 533.2 ± 23.5 | 340.7 ± 26 |
| Day 18 | 961.3 ± 61.9 | 761.9 ± 60.9 | 420.6 ± 20.2 | 259.2 ± 23.5 | 403.8 ± 32.2 | 274.7 ± 26.1 | 985.6 ± 86.7 | 639.9 ± 39 | 371.8 ± 28.8 |
| Day 21 | 1082.1 ± 74.5 | 891.2 ± 82.8 | 514.2 ± 16.5 | 235.9 ± 18.3 | 470.3 ± 29.7 | 261.1 ± 29.6 | 1056.1 ± 83.2 | 744.8 ± 47.7 | 399.8 ± 32.4 |
| Day 25 | 1205.8 ± 68.3 | 1005.9 ± 71.4 | 596.9 ± 22.9 | 232.9 ± 21.3 | 538.6 ± 24.1 | 240.8 ± 32.5 | 1184.9 ± 82.6 | 833.1 ± 42.4 | 406.3 ± 33.2 |

In this study, the antitumor effect of the compound of formula (I) (10 mg/kg or 30 mg/kg PO QD), enzalutamide (20 mg/kg PO QD), abiraterone (350 mg/kg PO QD), and combinations thereof, on non-castrated Vcap prostate cancer model in male CB17/Scid mice was evaluated. The vehicle control group reached a tumor size of 120.8 mm³. All treatment groups reduced mean tumor volume. The greatest inhibition of tumor growth was achieved by treatment with the compound of formula (I) (30 mg/kg) monotherapy. Comparable inhibition was achieved by treatment with the dual combination of the compound of formula (I) and enzalutamide (20 mg/kg).

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

1. A method of treating a cancer selected from metastatic breast cancer (mBC), metastatic castration-resistant prostate cancer (mCRPC), and a high-grade glioma in a subject in need thereof, comprising administering to the subject a compound of formula (I) 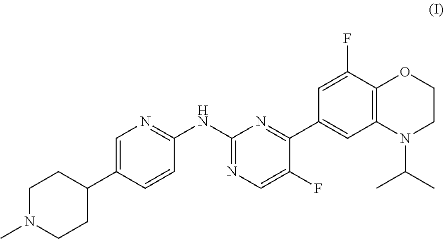 [SMILES: CC(C)N1CCOc2c(F)cc(-c3nc(Nc4ccc(C5CCN(C)CC5)cn4)ncc3F)cc21]
or a pharmaceutically acceptable salt thereof.

2. The method of claim 1, wherein the cancer is mBC.

3. The method of claim 2, wherein the mBC is hormone receptor-positive (HR+).

4. The method of claim 2 or 3, further comprising administering to the subject an anti-estrogen therapy.

5. The method of claim 4, wherein the anti-estrogen therapy is a selective estrogen receptor degrader (SERD), selective estrogen receptor modulator (SERM), estrogen receptor downregulator (ERD), or an aromatase inhibitor.

6. The method of claim 5, wherein the anti-estrogen receptor is a SERD.

7. The method of claim 6, wherein the SERD is fulvestrant.

8. The method of claim 7, wherein the fulvestrant is administered intramuscularly at a once or twice monthly dose from about 100 mg to about 500 mg.

9. The method of any one of claims 1 to 8, wherein the subject previously received an anti-estrogen therapy.

10. The method of any one of claims 2 to 9, wherein the cancer is resistant to or has become resistant to anti-estrogen therapy.

11. The method of claim 10, wherein the anti-estrogen therapy is a SERD.

12. The method of claim 11, wherein the SERD is fulvestrant.

13. The method of any one of claims 2 to 12, wherein the mBC is human epidermal growth factor receptor 2-positive (HER2+).

14. The method of claim 13, further comprising administering a HER2 targeted therapy to the subject.

15. The method of claim 14, wherein the HER2 targeted therapy comprises an anti-HER2 antibody.

16. The method of claim 15, wherein the anti-HER2 antibody is trastuzumab.

17. The method of claim 14, wherein the HER2 targeted therapy comprises tucatinib.

18. The method of claim 14, wherein the HER2 targeted therapy comprises trastuzumab and tucatinib.

19. The method of claim 1, wherein the cancer is mCRPC.

20. The method of claim 19, further comprising administering to the subject an anti-androgen therapy with the compound of formula (I), or a pharmaceutically acceptable salt thereof.

21. The method of claim 20, wherein the anti-androgen therapy is a selected from abiraterone acetate, enzalutamide, apalutamide, and darolutamide.

22. The method of claim 21, wherein the anti-androgen therapy is enzalutamide.

23. The method of claim 22, wherein the enzalutamide is administered orally at a dose of from about 40 mg to about 160 mg daily.

24. The method of any one of claims 19 to 23, wherein the subject previously received one or two anti-androgen therapies.

25. The method of claim 24, wherein the anti-androgen therapies are selected from abiraterone acetate, enzalutamide, apalutamide, and darolutamide.

26. The method of claim 24 or claim 25, wherein the patient's cancer is resistant to anti-androgen therapy.

27. The method of claim 26, wherein the anti-androgen therapy is enzalutamide.

28. The method of any one of claims 19 to 27, wherein the subject received prior taxane therapy.

29. The method of claim 1, wherein the cancer is a high-grade glioma.

30. The method of claim 29, wherein the high-grade glioma is glioblastoma.

31. The method of claim 29 or 30, wherein the high-grade glioma is characterized by a CDKN2A mutation.

32. The method of any one of claims 29 to 31, further comprising administering temozolomide to the patient.

33. The method of any one of claims 29 to 32, wherein the patient received prior treatment with temozolomide.

34. The method of any one of claims 29 to 33, wherein the cancer is resistant to temozolomide.

35. The method of any one of claims 1 to 34, wherein the compound of formula (I), or a pharmaceutically acceptable salt thereof, is administered orally.

36. The method of claim 35, wherein the compound of formula (I), or a pharmaceutically acceptable salt thereof, is administered once daily at a dose of from about 25 mg to about 250 mg.

37. The method of claim 36, wherein the compound of formula (I), or a pharmaceutically acceptable salt thereof, is administered once daily at a dose of about 100 mg.

38. The method of claim 36, wherein the compound of formula (I), or a pharmaceutically acceptable salt thereof, is administered once daily at a dose of about 150 mg.

39. The method of claim 36, wherein the compound of formula (I), or a pharmaceutically acceptable salt thereof, is administered once daily at a dose of about 200 mg.

40. The method of any one of claims 1 to 39, wherein the subject received prior treatment with a CDK4/6 inhibitor.

41. The method of any one of claims 1 to 40, wherein the subject is resistant to or has become resistant to a CDK4/6 inhibitor.

42. The method of claim 40 or 41, wherein the CDK4/6 inhibitor is selected from palbociclib (Ibrance®), ribociclib (Kisqali®) and abemaciclib (Verzenio®).

43. The method of any one of claims 1 to 42, wherein the cancer has metastasized to brain.

44. A method of treating brain metastases in a subject in need thereof, comprising administering to the subject a compound of formula (I) 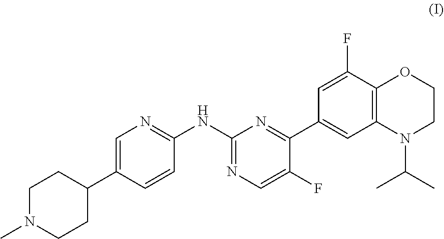 [SMILES: CC(C)N1CCOc2c(F)cc(-c3nc(Nc4ccc(C5CCN(C)CC5)cn4)ncc3F)cc21]
or a pharmaceutically acceptable salt thereof.

45. A method of preventing the growth or survival of metastasizing cancer cells in the brain of a subject in need thereof, comprising administering to the subject a compound of formula (I) 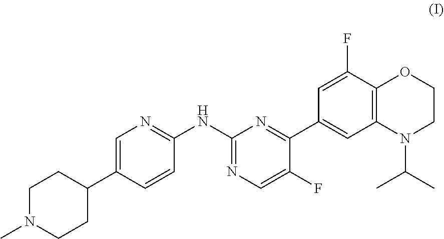 [SMILES: CC(C)N1CCOc2c(F)cc(-c3nc(Nc4ccc(C5CCN(C)CC5)cn4)ncc3F)cc21]
or a pharmaceutically acceptable salt thereof.

46. The method of claim 44 or 45, wherein the subject has a primary cancer selected from breast cancer, colon cancer, lung cancer, melanoma and leukemia.

47. The method of claim 46, where the primary cancer is breast cancer.

48. The method of claim 47, wherein the breast cancer is HR+.

49. The method of claim 47 or 48, wherein the breast cancer is HER2+.

50. The method of any one of claims 1 to 49, wherein the subject has a mutation in the Estrogen Receptor 1 (ESR1) gene.

51. The method of claim 50, wherein the mutation is Y537S.

52. The method of any one of claims 1 to 51, wherein the subject has a mutation in the CDKN2A gene.
